(19) United States
(12) Patent Application Publication
(10) Pub. No.: US 2017/0020402 A1
(43) Pub. Date: Jan. 26, 2017

(54) IMPLANTABLE AND BIORESORBABLE SENSORS

(71) Applicants: The Board of Trustees of the University of Illinois, Urbana, IL (US); Washington University, St. Louis, MO (US)

(72) Inventors: John A. ROGERS, Champaign, IL (US); Rory KJ MURPHY, St. Louis, MO (US); Seung Kyun KANG, Urbana, IL (US); Seung Min LEE, Champaign, IL (US); Daniel Vincent HARBURG, Urbana, IL (US); Wilson Z. RAY, St. Louis, MO (US)

(21) Appl. No.: 15/146,629

(22) Filed: May 4, 2016

Related U.S. Application Data
(60) Provisional application No. 62/156,795, filed on May 4, 2015.

(51) Int. Cl. A61B 5/03; A61B 5/00; A61B 5/11; A61B 5/022; A61B 5/01; A61B 5/145

(52) U.S. Cl. CPC A61B 5/031; A61B 5/01; A61B 5/14532; A61B 5/14539; A61B 5/11; A61B 5/14542; A61B 5/022; A61B 5/6868; A61B 2503/40; A61B 2503/42; A61B 2562/0209; A61B 2562/125; A61B 2562/0261; A61B 2560/0223

(57) ABSTRACT

Provided herein are implantable and bioresorbable sensors that are specifically designed to be implanted, function for an operational lifetime, and at some point after the operational lifetime to be resorbed by the body in such a manner so as to avoid having to remove any part of the sensor. This avoids a second impact on the patient otherwise associated with sensor removal. The sensors have a substrate and an electronic device supported by the substrate. A barrier layer isolates the electronic device from surrounding environment, including a biofluid, during use. Each of the substrate, electronic device, and barrier layer have a bioresorption rate during use to provide controlled bioresorption such reliable measure of one or more parameters occur over and an operational lifetime, and after a bioresorption lifetime greater than the operational lifetime, no detectable portion of the implantable and bioresorbable sensor remains at an implantation site.

### CROSS-REFERENCE TO RELATED APPLICATIONS

This application claims the benefit of U.S. Provisional Patent Application No. 62/156,795 filed May 4, 2015, which is specifically incorporated by reference to the extent not inconsistent herewith.

### STATEMENT REGARDING FEDERALLY SPONSORED RESEARCH OR DEVELOPMENT

This invention was made with U.S. government support under W911NF-11-1-0254 and D131-005-0070 awarded by DARPA and 1242240 awarded by the NSF. The U.S. government has certain rights in the invention.

### BACKGROUND

Provided herein are biomedical devices related generally to implantable and fully bioresorbable sensors that sense one or more physiologically-relevant parameters for a desired operational lifetime. After the operational lifetime, the sensor and related components dissolve and are resorbed by the body. Related methods for making, implanting, and using the devices described herein are provided.

Implantable biomedical sensors have potential for a range of important clinical applications, such as treatment and/or monitoring of traumatic brain injury, neurological disorders (e.g., epilepsy and Parkinson's disease), heart disorders (e.g., arrhythmias), vascular disorders, orthopedic disorder, muscular and/or nerve disorders and therapeutic and efficacy assessments thereafter. Efficacious use of implantable biomedical devices, however, is constrained in part upon design strategies that provide an avenue for complications, ranging from infection to adverse immune response due to bio-incompatibility issues between the implanted device and the body's immune system. Overcoming these challenges is important because of the numerous benefits that would be realized by having detailed information about an otherwise difficult to access, assess and measure parts of the body.

The brain, in particular, has unique challenges that make pressure monitoring of particular interest. Intracranial pressure (ICP) is the pressure inside the skull and it is important that ICP is maintained within a relatively narrow pressure range. Outside this range, severe harm to the brain is likely. A highly damaging aspect of traumatic brain trauma is elevated ICP, with a high correlation to a poor patient outcome. In particular, intracranial hematoma or cerebral edema can, if not quickly relieved, impact brain tissue and cause related adverse brain abnormalities with associated severe complications. Accordingly, there is a need to quickly, reliably, and minimally invasively monitor ICP, including during traumatic brain surgery and recovery.

Recently, a number of patents and publications have disclosed implantable, biodegradable devices. For example, International Patent Application Publication WO 2008/085904 discloses biodegradable electronic devices that may include a biodegradable semiconducting material and a biodegradable substrate. International Patent Application Publication WO 2008/108838 discloses biodegradable devices for delivering fluids and/or biological material to tissue. International Patent Application Publication WO 2008/127402 discloses biodegradable sensors containing embedded biological materials. International Patent Application Publication WO 2008/103464 discloses medical devices having nanostructured surfaces, which are optionally coated with a biodegradable polymer. Similarly, International Patent Application Publication WO 99/45860 discloses devices having biocompatible, and optionally resorbable, substrates with projections that, depending on their spacing, either promote or discourage cell adhesion.

Other patents and publications have disclosed implantable electronic devices. For example, U.S. Pat. No. 5,403,700 discloses devices having polyimide substrates supporting patterned metal conductors. U.S. Pat. No. 7,190,051 discloses hermetically packaged and implantable electronics fabricated using silicon-on-insulator technology. International Patent Application Publications WO 2009/111641 and WO 2009/114689 disclose stretchable and flexible electronic devices and sensor arrays, and WO2013/154606 and 2013/041973 describe intracranial pressure systems.

### SUMMARY

A challenge with monitoring biologically-relevant physical parameters within a body relates to the inherent invasiveness of the procedure. The act of physically inserting a monitoring device into the body to accurately and reliably monitor a parameter of interest and then actively removing the device from the body has a number of disadvantages, including the risk of infection. This risk is particularly relevant for any devices that are transcutaneous, with a direct physical path from outside the body to inside the body. In addition, conventional implanted devices for temporary monitoring of physical parameters generally require a second surgical procedure to remove the implanted device from the patient.

These challenges are addressed herein by implanting a fully resorbable device into the patient, monitoring the parameter(s) over an operational lifetime and wirelessly transmitting the data associated with the monitored parameter to a receiver unit outside the body. The device then fully resorbs. The fully resorbable device avoids the need for any subsequent intervention to remove the device and the wireless transmission minimizes risk of infection by avoiding an access route into the body. Accordingly, particularly useful applications include implantation into not readily-accessible regions, including inside the skull and adjacent to the brain, where ICP can be monitored. The ability to obtain the monitored parameter(s) at a distance also is advantageous for permitting rapid patient discharge after a procedure as the monitored parameter(s) may still be transmitted remotely from the patient's home, with an alarm and attendant rapid reaction response and intervention for any monitored parameter(s) outside a user-selected tolerance range.

Provided herein are implantable and bioresorbable sensors comprising: a substrate; an electronic device supported by the substrate, wherein the electronic device comprises a semiconductor or metallic component; a barrier layer that isolates the electronic device from a surrounding environment during use. The surrounding environment may comprise a tissue and/or biofluid. The isolation may be further described in terms of isolating the device from one or more of a variety of parameters, such as fluidic isolation (e.g., quantified by permeability), electric isolation (e.g., quantified by current leakage) and/or chemical isolation (e.g., quantified by degradation rate, leakage of chemical species) of the electronic device from the surrounding environment, including a biofluid, during use. Each of the substrate, electronic device, and barrier layer have a bioresorption rate during use configured so that the implantable and bioresorbable sensor operates for an operational lifetime and has a bioresorption lifetime that is greater than the operational lifetime. The bioresorption rate may be time-varying, with an initial low level resorption that may increase with time, particularly as environmental factors leak through the barrier and/or encapsulation layers, or as the barrier and/or encapsulation layers themselves degrade. In this manner, no detectable portion of the implantable and bioresorbable sensor remains at an implantation site after the bioresorption lifetime.

Examples of operational lifetimes include, depending on the application of interest, greater than about one hour, from between 8 hours to one year, between 1 day and one year, between 1 day and four months, and any subranges thereof. The bioresorption lifetime may be described in terms of an excess time beyond the operational lifetime, such as at least an additional 1 hour, at least an additional 8 hours, at least an additional 1 day, or at least an additional week beyond the operational lifetime. The ability to configure the sensor to have any of a range of operational and bioresorption timeframes and time-courses is one advantage of the instant sensors, making them widely applicable to various tissues, body compartments and organs.

The sensors are compatible with a range of materials and geometries. For example, the semiconductor or metallic component may be described as having a thickness that is relatively thin, so as to facilitate implantation without damaging biological tissue as well as desired bioresorption rates and corresponding operational lifetime. For example, the thickness may be less than or equal to 10 μm, less than or equal to 1 μm, between 1 nm and 10 μm, between 10 nm and 10 μm, or any subrange thereof. Such thicknesses are useful for providing desired functionality, including a providing a desired operational lifetime and, after the operation lifetime, a desired bioresorption lifetime, as thinner layers generally will bioresorb over a shortly lifetime compared to corresponding thicker layers.

The sensors provided have a wide range of functionality with respect to sensor type and physical parameters being monitored, with specific configurations dependent on the application of interest, including the location of the implant and the desired parameter(s) to be monitored. For example, for brain-related applications including traumatic brain injury and surgical interventions, pressure may be an important parameter of interest. As desired, additional parameters of interest may include electric potential, blood flow, pH and temperature. The sensors and related methods provided herein are particularly advantageous in that they may be designed to monitor any number or types of physical parameters, while avoiding the need for removal of the sensor.

Examples of electronic devices include those having a sensor selected from the group consisting of: a pressure sensor; a temperature sensor; a motion sensor; a flow sensor; a thermal conductivity sensor; a diffusivity sensor; a pH sensor; an electrical sensor; an optical sensor; a glucose sensor, oxygen sensor and a biomolecule sensor. For multi-functionality, the electronic device may comprise a first electronic device that senses a first physical parameter and a second electronic device that senses a second physical parameter, wherein the physical parameters are selected from the group consisting of pressure, temperature, acceleration, electric potential, fluid flow-rate, thermal conductivity, pH, glucose level, oxygen level, and biomolecule detection. The electronic devices may be separately and uniquely positioned relative to the substrate, with the output from each sensor of the electronic device electronically communicated to a device outside the patient, such as for analysis by medical personal or the patient

The implantable and bioresorbable sensor may have an electronic device comprising a pressure sensor, with a cavity disposed in the substrate; a deformable layer supported by the substrate and that covers the cavity; and a strain gauge supported by the deformable layer and adjacent to the cavity. In this configuration, deflection of the deformable layer by a pressure in a fluid surrounding or adjacent to the deformable layer and strain gauge generates an electrical output from the pressure sensor strain gauge that depends on the fluid pressure. The strain gauge may be positioned on a top surface of the deformable layer, so that the deformable layer is between the strain gauge and the cavity, or on a bottom surface of the deformable layer so that the strain gauge is between the deformable layer and the cavity.

The strain gauge is made from any material that is capable of outputting an electric parameter that is dependent on the strain. Examples include a range of semiconductor and metal materials, such as a Si-nanomembrane; a polycrystalline Si; a metal or alloy thereof, including metals such as Mg, Mo, Zn, W, Fe. The active distal sensing end of the strain gauge may be arranged in a serpentine configuration.

The deformable layer may be formed from a range of materials, including a polymer or elastic-type material, or a thin inorganic material. Functionally, the deformable layer should reliably and repeatedly deform in a manner that is proportional to fluid pressure difference between the underlying cavity in the substrate and fluid that is on top of the deformable layer (e.g., outside the cavity with the deformable layer positioned therebetween). Examples of polymer materials include, but are not limited to, one or more of: poly(lactic-co-glycolic acid) (PLGA), polycaprolactone (PCL), poly glycolic acid (PGA), poly lactic acid (PLA), poly glycerol sebacate (PGS), agarose gel, polyanhydrides. Other examples of a deformable layer suitable for use herein includes collagen and rice paper, or an inorganic layer formed from a material selected from the group consisting of single crystalline silicon, polycrystalline silicon, amorphous silicon; silicon oxides, silicon nitrides, and a-IGZO, the inorganic layer having a thickness less than 10 μm.

The semiconductor component may comprise a layer of single crystalline Si, amorphous Si, polycrystalline Si, silicon oxide, silicon nitride, or any other Si based resorbable layer or semiconductors layer.

The metallic component may comprise a layer of Mg, Zn, W, Fe, Mo, or alloys thereof.

The strain gauge may be a Si-nanomembrane strain gauge having a distal piezoresistive end with a serpentine geometry. “Serpentine geometry” refers to a pattern of material that meanders or doubles back on itself in a parallel configuration, but in a manner where adjacent portions are physically separated.

The cavity may have any of a variety of shapes, geometry, and physical dimensions, so long as the deformable layer reliably deforms under a pressure differential, such as pressure exerted on a bottom surface facing the cavity and pressure exerted on a top surface facing the biological tissue or biofluid. The cavity may have a rectangular or a square shape with a depth that is greater than or equal to 20 μm and less than or equal to 100 μm, such as for a substrate thickness that is greater than 70 μm and less than 200 μm. The cavity may also be described in terms of a cavity volume, such as a cavity volume that is greater than 0.1 mm³ and less than 2 mm³.

As described herein, the implantable and bioresorbable sensor is compatible with a range of sensors for detecting various physical or biochemical properties. Examples include, but are not limited to, any one or more of a: motion sensor comprises a single-axis accelerometer; temperature sensor comprises a silicon-nanomembrane having a resistance that is temperature-dependent; flow sensor comprises a silicon nanomembrane having a thermal actuator positioned between adjacent temperature sensors, wherein a difference in detected temperatures between adjacent temperature sensors is proportional to a flow-rate over the adjacent temperature sensors; pH sensor comprises silicon nanoribbons and gate electrodes; biomolecule sensor comprises a surface-functionalized silicon-nanomembrane; electrical sensor comprises electrodes and an electrical circuit in electrical contact with the electrodes and formed of active and passive components selected from one or more of the group consisting of: diodes, transistors, resistors; and/or optical sensor comprises a photoelectronic device in electrical contact with a photosensitive component.

Also provided herein are implantable and bioresorbable sensors configured to provide wireless transmission. This is a particularly useful configuration in that the need for electrical connectors that traverse from the exterior of the patient to the interior of the patient is avoided. Such connectors are susceptible as a transmission means for unwanted organisms or other complications. Accordingly, any of the implantable and bioresorbable sensors may further comprise an at least partially resorbable wireless data-transmitter electrically connected to the electronic device.

The electronic device may further comprise: a near-field communication (NFC) microchip; an electronic circuit comprising: electrical interconnects, resistors, and capacitors that electrically connect the NFC chip and semiconductor or metallic component for data acquisition, processing and transmission; and an inductive coil or radiofrequency receiver for powering the implantable and bioresorbable sensor from an externally-positioned electronic component. Such powering means, or any future arising equivalent thereof, provides a platform for powering the device without physically connecting an external device to the implanted sensor. The sensor, of course, is compatible with future arising power means, such as biodegradable power sources and batteries.

The implantable and bioresorbable sensor may further comprise a bioresorbable wire to electrically connect the electronic device to a wireless transmitter for wirelessly transmitting data from the implantable and bioresorbable sensor to a location physically separated from the implantable and bioresorbable sensor.

The implantable and bioresorbable sensors provided herein may be further described in terms of one or more additional structural aspects, geometry, or form factor. For example, the substrate may have a thickness less than 100 μm and comprise: nanoporous silicon, single crystalline silicon, polycrystalline silicon, amorphous silicon; silicon oxides, silicon nitrides, amorphous indium gallium zinc oxide (a-IGZO), or other semiconductor and their oxides or nitrides; or a foil of magnesium, zinc, tungsten, iron, molybdenum, and their alloys thereof, such as a foil having a thickness less than about 10 μm.

The implantable and bioresorbable sensor may further comprise: a polymer layer supported by the substrate, wherein the polymer layer comprises any bioresorbable polymer including, but not limited to, poly(lactic-co-glycolic acid (PLGA), silk fibroin, polycaprolactone (PCL), poly glycolic acid (PGA), poly lactic acid (PLA), poly glycerol sebacate (PGS), collagen and rice paper, agarose gel, polyanhydrides, the polymer layer having a thickness less than 100 μm; an inorganic layer supported by the substrate, wherein the inorganic layer comprises single crystalline silicon, polycrystalline silicon, amorphous silicon; silicon oxides, silicon nitrides, or a-IGZO, the inorganic layer having a thickness less than 10 μm; or both the polymer layer and the inorganic layer supported by the substrate, including a polymer layer positioned between the substrate and the inorganic layer.

The polymer layer, inorganic layer, or both the polymer layer and inorganic layer may cover a cavity disposed in a portion of the substrate such that the polymer layer and/or the inorganic layer deflects with a magnitude that corresponds to a pressure in a fluid surrounding the implantable and bioresorbable sensor, and more particularly the pressure difference exerted by the fluid on the top-facing surface of the layer covering the cavity compared to the pressure exerted by air in the cavity on the bottom-facing surface of the layer. Accordingly, the cavity may be referred herein as an air cavity to reflect that the cavity has a substantially air-tight seal.

The barrier layer may comprise: silicon oxide or silicon nitride having a thickness less than 5 μm; or silk fibroin, polycaprolactone (PCL), poly glycolic acid (PGA), poly lactic acid (PLA), poly glycerol sebacate (PGS), collagen and rice paper, agarose gel, polyanhydrides, or a combination thereof, having a thickness less than 1 mm.

The implantable and bioresorbable sensor, as a whole, may have: a thickness that is less than 3 mm, a volume that is less than 5 cm³, and a total mass less than 10 g.

The implantable and bioresorbable sensor may further comprise an encapsulation layer that encapsulates the implantable and bioresorbable sensor, the encapsulation layer comprising a bioresorbable polymer layer, a bioresorbable inorganic layer, or both a bioresorbable polymer layer and a bioresorbable inorganic layer, wherein the bioresorbable polymer layer and the bioresorbable inorganic layer have a thickness selected to provide a desired operational lifetime. In this manner, for longer desired operating lifetime, the encapsulation layer may have a relatively larger thickness and/or be formed from a material having a lower dissolution or bioresorption rate. Furthermore, an encapsulation layer may be used to provide a desired mechanical property of the device, including a modulus and/or to protect sensitive portions of the sensor. As desired, the barrier layer itself may be configured to also function as an encapsulation layer. Alternatively, there may be distinctly configured and positioned layers that are a barrier layer(s) and an encapsulation layer(s). Use of multiple layers can provide further fine-control of degradation characteristics and operating lifetimes, including in a range of biological environments having a range of operation conditions.

Other functional characteristics of the barrier layer and/or encapsulation layer, includes limiting a net leakage current or heat from the electronic device to surrounding biological tissue to an amount which does not adversely affect the tissue. For example, the net leakage current from the electronic device may be limited to 10 μA or less, optionally for some applications 5 μA or less, or optionally for some applications 1 μA or 0.1 μA or less. The barrier layer may prevent leakage current from being concentrated to small areas so to prevent tissue damage caused by current leakage from the sensor. For example, the barrier layer may be configured to limit leakage current from the sensor to the biological environment to 0.1 μA/cm² or less, and for some applications 0.01 μA/cm² or less, and for some applications 0.001 μA/cm² or less. In some embodiments, barrier layers of the invention have an electrical resistivity of 10¹⁴ Ω·m or greater, for example an electrical resistivity selected over the range of 10¹⁵ to 10¹⁷ Ω·m. In some embodiments, the barrier layer prevents the rate at which charge is leaked from the electronic device; for example, one barrier layer embodiment limits electrical discharge from a device to 10 μC or less over a period of 1 second. In some embodiments, the barrier layer limits leakage current or average leakage current from the device to 10 μA or less or 5 μA or less over a long period of time, such as 3 hours or more or 5 hours or more.

The barrier and/or encapsulation layer may also function as a moisture barrier, limiting fluid permeation to underlying water-sensitive electronic currents.

The barrier and/or encapsulation layer(s) may also be described in functional terms, such as ensuring that one or both of a desired operational lifetime or bioresorption lifetime is achieved. For example, the layer(s) may be configured to provide a sufficiently low permeability so that hydrolysis of a hydrolytic-sensitive component does not occur until an operational lifetime is achieved. Similarly, permeability may be selected to be sufficiently high so that the component undergoes a desired amount of hydrolysis or corrosion to achieve a desired a bioresorption lifetime. This may be further described in terms of a dissolution rate, including a corrosion rate, and is further described in WO 2014/169218 (atty ref. 555286: 56-13 WO), which is specifically incorporated herein, for various materials with corresponding dissolution or corrosion rates.

The barrier layer may be constructed so as to be substantially impermeable, whereas the encapsulating layer may have some level of permeability, including a barrier layer that has less than 10%, less than 5%, or less than 1% of the permeability of the encapsulating layer. In this manner, each of the barrier layer and encapsulating layer may provide distinct functionality, and, when used in combination, a desired sensor characteristic achieved.

The implantable and bioresorbable sensor may have a bioresorbable polymer layer that comprises one or more of: silk fibroin, polycaprolactone (PCL), poly glycolic acid (PGA), poly lactic acid (PLA), poly glycerol sebacate (PGS), collagen and rice paper, agarose gel, polyanhydrides, polyglycolide, polyhydroxobutyrate, hyaluronic acid, hydrogels, poly(2-hydroxyethyl-methacrylate), poly(ethylene glycol), poly(dioxanone), poly(trimethylene carbonate), polyphosphazenes, chitosan, fibrin, gelatin or hyaluronan; and the bioresorbable inorganic layer comprises single crystalline Si, polycrystalline Si, amorphous Si, germanium, silicon-germanium, Si oxide, Si nitride, or any combination thereof.

Any of the implantable and bioresorbable sensors provided herein may further comprise an electrical interconnect; a wireless transmitter, wherein the electrical interconnect electrically connects the wireless transmitter to the electronic device; and the electrical interconnect comprises a bioresorbable metal wire having a thickness less than 50 μm and a width less than 500 μm.

The implantable and bioresorbable sensor may have an operational lifetime that is greater than or equal to 1 day, or one week, wherein for a bioresorption lifetime that is greater than the operation lifetime, each of the substrate, the barrier layer and the electronic device are configured for bioresorption by a patient in which the implantable and bioresorbable sensor is implanted.

The constituent parts of the implantable and bioresorbable sensor may be selected and configured so as to dissolve by any one or more of hydrolysis, enzyme reaction, or metabolic reaction after implantation in a programmed timeframe. Constituent parts includes the substrate, electronic device, barrier layer, and components thereof.

The constituent parts of the sensor may be configured to dissolve within 12 months after implantation, or any time period as desired post-implantation, including by designing the relative parts to have a desired dissolution rate. For example, the substrate may comprise nanoporous-Si or a metal foil configured to have a dissolution rate when in contact with a biofluid during use that is between 1 μm/day and 15 μm/day; the electronic device may comprise a Si nanomembrane configured to have a dissolution rate when in contact with a biofluid during use that is between 0.5 nm/day and 10 nm/day; and the barrier layer may comprise SiO₂ configured to have a dissolution rate when in contact with a biofluid during use that is between 0.1 nm/day and 5 nm/day. Similarly, with a known dissolution rate, the corresponding thickness may be selected so as to achieve a desired operational lifetime of the sensor, as the thicker the layer, the longer time for sufficient dissolution so as to result in sensor non-function and corresponding bioresorption.

The implantable and bioresorbable sensor may further comprise electrical interconnects to electrically connect the electronic device to a wireless transmitter, wherein the electrical interconnects comprise biodegradable metal wires having a thickness less than 50 μm and configured to have a dissolution rate when in contact with a biofluid during use, such as a dissolution rate that is between 0.2 nm/day and 20 nm/day. The biodegradable metal may be formed from any of a variety of metals and alloys thereof, including, but not limited to, molybdenum.

The implantable and bioresorbable sensor may further comprise a polymer layer supported by the substrate, the polymer layer configured to have a dissolution rate when in contact with a biofluid such that after a time period that is greater than three weeks the polymer layer dissolves.

The implantable and bioresorbable may have a barrier layer composition and a barrier layer thickness that is selected to provide a desired operational lifetime, such as an operational lifetime that is on the order of hours and greater, or between 1 day and 12 months.

After any one or more of hydrolysis, enzyme reaction, or metabolic reaction, the implantable and bioresorbable sensors are reduced to end products that may be configured to be absorbed by tissue adjacent to the implantable and bioresorbable sensor; configured to diffuse in a direction away from an implantation site; and/or configured to convect in a direction away from the implantation site; so that no detectable portion of the implantable and bioresorbable sensor remains at an implantation site.

The implantable and bioresorbable sensor may be configured so that no substantial immune response is observed post-implantation at an implantation site throughout a lifecycle of the implantable and bioresorbable sensor. No “substantial immune response” reflects that minimal and minor immune responses may be anticipated, including simply by the trauma associated with the implanting/injecting of the tissue, including a deep tissue injection. Any such immune response, however, is minimal and short-lived.

The sensors provided herein are compatible with a range of applications. For example, the implantable and bioresorbable sensor may be for temporary monitoring of physical parameters of interest following a medical procedure selected from the group consisting of: transplantation; cranial surgery; cardiac surgery; neurosurgery; trauma surgery; orthopedic surgery; vascular surgery; and ophthalmologic surgery. As described herein, a particular benefit of the sensors is avoiding the need for a second procedure to remove the sensor. Instead, the sensor is implanted, data transmitted wirelessly for use by the patient and/or medical caregiver, and after a pre-programmed operational lifetime, the sensor completely disappears from the implantation site.

Two or more parameters may be monitored, including parameters selected from the group consisting of: pressure; temperature; movement; fluid flow; conductivity; diffusivity; pH; oxygen level; electric potential; optical image; presence of a biomolecule of interest; glucose presence or concentration; and lactate presence or concentration.

The implantable and bioresorbable sensor may be configured for implantation into a cavity of a living animal, wherein after an operational lifetime, no detectable portion of the implantable and bioresorbable sensor remains in the living animal.

The implantable and bioresorbable sensor may be configured to adhere to a tissue, body cavity or organ wall selected from the group consisting of: a luminal facing blood vessel wall; an external-facing blood vessel wall, brain, heart, lung, eye, esophagus, stomach, sphincter, liver, urinary bladder, and spinal cord.

To facilitate implantable and bioresorbable sensor implantation, the sensor may have a sharp-edged leading tip configured to minimize tissue damage during a deep tissue injection or implantation.

Any of the implantable and bioresorbable sensors may be a pressure sensor. For example, the implantable and bioresorbable pressure sensor may comprise: a substrate; a cavity disposed in the substrate; a deformable layer supported by the substrate and that covers the cavity; an electronic device supported by the substrate, wherein the electronic device comprises a strain gauge supported by the deformable layer and adjacent to the cavity, wherein deflection of the deformable layer and/or strain gauge by a fluid pressure in a fluid surrounding the top surface of the deformable layer and strain gauge generates an electrical output from the strain gauge that depends on a magnitude of the fluid pressure (e.g., pressure difference outside the cavity and inside the cavity); and a barrier layer that fluidically isolates the electronic device during use. After an operational lifetime, the components may dissolve and/or bioresorb, thereby automatically disappearing without having to take any active steps to remove the sensor.

The deformable layer comprises a polymer layer, an inorganic layer, or both a polymer and inorganic layer. The polymer layer may be formed from one or more of: poly(lactic-co-glycolic acid) (PLGA), silk fibroin, polycaprolactone (PCL), poly glycolic acid (PGA), poly lactic acid (PLA), poly glycerol sebacate (PGS), collagen and rice paper, agarose gel, or polyanhydrides. The inorganic layer is formed from one or more of single crystalline Si, amorphous Si, polycrystalline Si, silicon oxide, or silicon nitride.

The strain gauge may comprise a bioresorbable nanomembrane formed of: Si, polycrystalline Si, Mg, Mo, Zn, W, Fe, or alloys thereof, wherein the nanomembrane has a thickness less than 1 μm.

The implantable and bioresorbable pressure sensor may have a Si-nanomembrane strain gauge that comprises a distal piezoresistive end having a serpentine geometry, including so as to provide improved sensitivity over a range of pressures.

The implantable and bioresorbable pressure sensor may further comprise an additional sensor electrically connected to the electronic device to provide a multi-functional sensor that detects pressure and at least one additional physical parameter.

Also provided are various methods of using and methods of making any of the sensors described herein. For example, provided is an extraction-free method of remotely sensing one or more physical parameters in a living animal, including by: inserting into a living animal an implantable and bioresorbable sensor operably connected to a wireless transmitter; electrically coupling the inserted implantable and bioresorbable sensor to an externally located electronic component to power the sensor and wireless transmitter; outputting to the wireless transmitter from the implantable and bioresorbable sensor an electrical output, wherein the electrical output depends on the one or more physical parameters; wirelessly transmitting the electrical output to the externally located electronic component; and hydrolyzing the implantable and bioresorbable sensor so that after an operational lifetime no observable or detectable portion of the implantable and bioresorbable sensor remains in the living animal, thereby providing the extraction-free method of remotely sensing one or more physical parameters.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1K. Bioresorbable, silicon-based mechanical/physical/chemical sensors for biomedical applications. FIG. 1A, Schematic illustration of a biodegradable pressure sensor. The inset shows the location of the silicon-nanomembrane (Si-NM) strain gauge. FIG. 1B, Optical micrograph of the strain-gauge region. (Trench BD represents the boundary of trench) FIG. 1C, Image of a fully assembled implantable and bioresorbable sensor, including with a barrier layer. The outer diameter of the hypodermic needle is 1 mm. FIG. 1D, Left, distribution of principal strains across the PLGA layer, including the Si-NM strain gauge at the left edge, determined from finite element analysis (FEA) for an external pressure of 50 mm Hg. Right, corresponding displacement profile evaluated along the red dotted line in the left frame. (∈ₘₐₓ and d_{z} represent principal strain and vertical displacement, respectively.) FIG. 1E, Responses of a commercial pressure sensor (blue) and a calibrated biodegradable device (red) to time-varying pressure over a range relevant to intracranial monitoring. FIG. 1F, Response of a similar biodegradable device (red-top line), but configured as an accelerometer, with comparison to a commercial sensor (blue—bottom line). FIG. 1G, Comparison of the calibrated response of such a bioresorbable temperature sensor (red) to a commercial device (blue). FIG. 1H, The difference in temperature measured by two separate Si-NM temperature sensors placed near a Si-NM element for Joule heating allows assessment of flow rate. FIG. 1I, A single serpentine Si-NM used as both a temperature sensor and a heating element allows measurements of thermal conductivity and heat capacity. The graph shows time-dependent changes in temperature upon actuation of Joule heating in devices immersed in different liquids. The coefficients of thermal conductivity (K) of hexane, toluene, ethylene glycol, and water are 0.12, 0.13, 0.26, and 0.60 W m⁻¹ K⁻¹, respectively. FIG. 1J, When the Si-NM is exposed to aqueous surroundings, its conductance depends on pH. The graph shows measurements for immersion in solutions with pH values between 2 and 10. FIG. 1K, Images collected at several stages of accelerated dissolution of a bioresorbable pressure sensor upon insertion into an aqueous buffer solution (pH 12) in a transparent PDMS enclosure at room temperature.

FIGS. 2A-2E. Bioresorbable interfaces between intracranial sensors and external wireless data-communication modules with transdermal wiring. FIG. 2A, Image of bioresorbable pressure and temperature sensors integrated with dissolvable metal interconnects (sputtered molybdenum, Mo, 2 μm thick) and wires (Mo, 10 μm thick). The inset shows an optical micrograph of the serpentine Si-NM structures that form the sensing regions. The Si-NM that is not above the air cavity (left) responds only to temperature; the one at the edge and adjacent to the air cavity (right) responds primarily to pressure. FIG. 2B, Diagram of a bioresorbable sensor system in the intracranial space of a rat, with electrical interconnects that provide an interface to an external wireless data-transmission unit for long-range operation. FIGS. 2C-2D, Demonstrations of FIG. 2C, an implanted bioresorbable sensor in a rat, and FIG. 2D, a sutured individual. A thin film of PLGA (˜80 μm) and a degradable surgical glue (TISSEAL) seal the craniectomy defect to close the intracranial cavity. FIG. 2E, Healthy, freely moving rat equipped with a complete, biodegradable wireless intracranial sensor system.

FIGS. 3A-3F. Wireless measurement of intracranial pressure and temperature with bioresorbable sensors implanted in live, freely moving animals. FIGS. 3A-3E, Red, data from a transient, bioresorbable sensor; blue, data from a commercial sensor. FIG. 3A, Real-time wireless measurements of ICP, showing transient increases due to periodic motion induced by the Valsalva maneuver. FIG. 3B, In vivo observation of changes in ICP as a function of time in the Trendelenburg and reverse Trendelenburg positions. ICP increases in the 30° head-down position (Trendelenburg) as compared with the supine position, and decreases in the 30° head-up position (reverse Trendelenburg). FIG. 3C, Gradual increase and FIG. 3D, decrease in ICT due to application of a heating/cooling blanket. FIG. 3E, Measurements of ICP over three days reveal consistent responses from devices encapsulated with biodegradable polyanhydride. FIG. 3F, Confocal fluorescence images of the cortical surface beneath the dissolved device at 2, 4 and 8 weeks, illustrating the absence of inflammatory responses. The images are double-immunostained for GFAP (glial fibrillary acidic protein) to detect astrocytes (red), and Iba1 (ionized calcium-binding adaptor molecule 1) to identify microglia/macrophages (green). The dashed line indicates the site of the implant.

FIGS. 4A-4E. Application of bioresorbable sensors to various body cavities, and demonstration of an injectable format for deep brain monitoring. Red, data from a transient biodegradable sensor; blue (circles), data from a commercial sensor. FIGS. 4A-4B, Pressures measured in FIG. 4A, intra-abdominal and FIG. 4B, leg cavities. FIG. 4C, Image showing in vivo injection of a needle-shaped biodegradable pressure sensor (using a magnesium foil support, ˜80 μm thick) into the brain parenchyma with a stereotactic frame and arm. The inset shows a biodegradable pressure sensor inserted into hydrogel, as evidence of the sensor's robust mechanical construction. FIG. 4D, In vivo measurements of pressure in the deep brain. FIG. 4E, In vivo measurements of temperature in the deep brain during anaesthesia. The temperature drops during anaesthesia owing to reduced blood circulation, and returns to normal after awakening.

FIGS. 5A-5H. Fully implantable near-field communication (NFC) system with bioresorbable interface and intracranial sensors. FIG. 5A, Diagram of a fully implantable NFC system. This device uses a magnesium foil (˜50 μm) for the inductive coil, interconnects and electrodes; patterned silicon nanomembranes (Si-NMs, ˜300 nm) for resistors; conventional capacitors; and an advanced NFC microchip for data acquisition, processing and transmission. PLGA serves as the substrate and for encapsulation. The diameter of the entire device is about 15 mm. FIG. 5B, Image of this type of NFC system integrated with a bioresorbable pressure sensor. FIG. 5C, Diagram of the operational principles. FIG. 5D, Series of images showing accelerated dissolution of the NFC system inserted into an ACSF at 60° C. FIG. 5E, Diagram of the implantation process. The bioresorbable sensors reside in the intracranial space, while the NFC system is located extracranially, on the outside surface of the skull, beneath the skin. Bioresorbable, thin metal wires interconnect the NFC system and the sensors. FIG. 5F, Real-time wireless measurements of ICP, showing transient increases due to periodic motion induced by the Valsalva maneuver (red, data obtained from a transient ICP sensor; blue, data obtained from a commercial ICP sensor). FIG. 5G, Increase in ICT owing to application of a heating blanket around the head, as determined by bioresorbable (red) and commercial (blue) sensors. FIG. 5H, Demonstrations of implantation and suturing in a rodent model. A biodegradable surgical glue (TISSEAL) seals the intracranial space.

FIG. 6. Schematic cross-sectional side view of an implantable and bioresorbable pressure sensor.

FIGS. 7A-7B. Scanning electron microscope (SEM) images of nonporous Si. FIG. 7A, Cross-section view and FIG. 7B, top view of np-Si structure with ˜71% porosity.

FIG. 8. Biodegradable pressure sensor with Mg trench structure. Trench depth and thickness of Mg foil are ˜40 μm and ˜80 μm, respectively.

FIG. 9. Image of a bioresorbable pressure sensor, with a thickness of ˜110 μm, a weight of ˜1 mg and overall lateral dimensions of 3 mm×6 mm and trench dimensions of 2 mm×2.4 mm×40 μm.

FIG. 10. Optimization of the location of the piezoresistive serpentine sensors.

FIG. 11. Optimization of trench geometry through stress-strain analysis using the finite element method (FEM). Top-left panel shows full image of simulated dimension; principle strain distribution around the piezoresistive serpentine sensors for various trench geometries. ∈ₐ represents average strain on coils.

FIG. 12. Piezoresistive response of the pressure sensor compared to finite element method (FEM) simulation (error bars represents standard deviations).

FIG. 13. Calibration curve of pressure sensor with 2 mm×2.4 mm×40 μm dimension.

FIG. 14. In vitro test of transient pressure sensor in ACSF solution.

FIGS. 15A-15C. Calibration of resistance change to FIG. 15A, pressure, FIG. 15B, acceleration, and FIG. 15C, temperature.

FIGS. 16A-16B. Transient accelerometer with Si-NM piezoresistive strain sensor. FIG. 16A, Materials and structure of accelerometer with PLGA proof mass. FIG. 16B, Image of bioresorbable accelerometer on the Si structure.

FIGS. 17A-17B. Bioresorbable temperature sensor on a thin PLGA film. FIG. 17A, Materials and structure of a thermoresistive Si temperature sensor. FIG. 17B, Microscope image of a dense serpentine Si-NM structure for enhanced thermoresistive response.

FIG. 18. In vitro setup for transient temperature sensors.

FIG. 19. Principle of flow rate monitor based on a thermal actuator and a pair of temperature sensors. The difference between the temperatures recorded at the two temperature sensors (T₂−T₁) increases as the flow rate increase.

FIG. 20. Bioresorbable pH sensor constructed with Si-NRs, Mg electrodes and PLGA substrates.

FIG. 21. Hydrolysis kinetics of materials used in the bioresorbable pressure sensors. Normalized thickness (h/h₀) as a function of time during dissolution of individual materials in artificial cerebrospinal fluid (ACSF) at physiological temperature (37° C.). The initial thicknesses are 200 nm for Si nanomembranes (Si NMs), 80 μm for porous Si (p-Si), 80 μm for Mg foil, and 100 nm for SiO₂.

FIG. 22. Schematic diagram (left) and image (right) of PDMS structure used as a simple mimic of the intracranial space.

FIGS. 23A-23B. Scanning electron microscope (SEM) images of FIG. 23A, Si NMs and FIG. 23B, np-Si at the different stages of dissolution in buffer solution with pH 12 at 37° C.

FIGS. 24A-24B. Calibration of the temperature dependent piezoresistivity. FIG. 24A, Resistivity variation to applied pressure at various temperatures (error bars represents standard deviation). FIG. 24B, Variation of sensitivity of resistivity to pressure associated with changes in temperatures. The change of piezoresistivity sensitivity is negligible across the expected range of brain temperatures.

FIG. 25. Strategy for interconnection between a bioresorbable device and degradable external wires on biodegradable polymer. Transfer printing the biodegradable wires (Mg or Mo) on the biodegradable polymer substrate (PLGA), and depositing dissolvable metal (Mo) between the wires and sensors yield the fully bioresorbable interface.

FIG. 26. Image of the interface between the bioresorbable wires and the wireless transmitter.

FIGS. 27A-27B. Effect of encapsulation on the response of the pressure sensor; in this example a polyanhydride encapsulant. FIG. 27A, Calibration curves before and after encapsulation. The calibration factor changes from 82 to 50 Ω/mmHg with 120 μm thick encapsulation (error bars represents standard deviation). FIG. 27B, The thickness dependent sensitivity simulated by FEM.

FIG. 28. Chemistry of synthesis and hydrolysis of a biodegradable polymer (class of polyanhydride) for encapsulation.

FIG. 29A, Dissolution kinetics and FIG. 29B, water permeability (tested by 300 nm thick Mg resistor) of polyanhydride encapsulation.

FIGS. 30A-30B. Hematoxylin and eosin (H&E) images of tissue around the implant site of FIG. 30A polyanhydride and FIG. 30B HDPE after 14 days.

FIG. 31. In vitro operation of bioresorbable pressure sensor with functional lifetime controlled with a biodegradable encapsulation layer.

FIGS. 32A-32B. Effect of dissolution of metal wires and interconnects on the resistance. FIG. 32A, Increases in resistance of biodegradable metal wire (Mo, 10 μm; Mg, 50 μm) in ACSF at physiological temperature for about a week. The change in resistance is negligible (below a few ohms) for a week. The resistance of Mg wire rapidly increases after 7 days due to its higher dissolution rate compared to Mo. FIG. 32B, Changes in resistance of Mo interconnections (˜2 μm) during hydrolysis in ACSF at body temperature.

FIG. 33. Resistance measurement of a pressure sensor with polyanhydride encapsulation with constant external pressure. The deviation of the resistance after about one week provides an indication of the operational lifetime of the pressure sensor.

FIGS. 34A-34B. In vitro and in vivo demonstrations of biodegradable temperature sensors. FIG. 34A, Calibration curves for resistance to temperature indicate stable operation over 6 days in ACSF. FIG. 34B, Stable in vivo operation is demonstrated for three days. Sensors are encapsulated with a 120 μm thick layer of polyanhydride.

FIGS. 35A-35C. Confocal fluorescence images of the cortical surface. FIG. 35A, Series of images of the sham area (left side of brain) and the area underneath the np-Si pressure sensor (right side of brain). FIG. 35B, Image of the cortical surface at the site implanted with a Mg supported device and FIG. 35C, at the sham site.

FIG. 36. Animal behavior evaluation with transcutaneous wire implantation using novel object recognition test.

FIGS. 37A-37B. Frequency dependent FIG. 37A, impedance and FIG. 37B, phase of the NFC system at the different distance (black line; 10 mm, red dot; 10 mm with 2 mm barrier of phosphate buffer solution (PBS), blue line; 15 mm, cyan line; 20 mm, magenta line; 25 mm).

FIG. 38. IR thermography of an NFC system during wireless operation in air.

FIGS. 39A-39D. High sampling rate of NFC system. FIG. 39A, Wirelessly transmitted voltage sine waves with frequencies between 1 to 50 Hz. Maximum sampling rate of this system is 250 Hz. FIG. 39B, Spectrogram of swept sine wave in FIG. 2F. High speed data acquisition of NFC system demonstrated with FIG. 39C, sine and FIG. 39D, square wave of 10 Hz frequency. (Red dot line is the data point measured by NFC system and blue line is input signal from signal generator.)

FIG. 40. Gain response of programmed real-time high (red) and low (blue) pass filtering, performed by the NFC chip, as a function of frequency.

FIG. 41. Comparison of filtered (red) and unfiltered (black) gain during pressure measurement. Two channels are measured through NFC system at the same time. The filter function is loaded and performed in the chip.

FIG. 42. Response of a commercial pressure sensor (blue) and a wireless, biodegradable system (red) to time-varying pressure over a range relevant to intracranial monitoring.

FIGS. 43A-43B. Geometry of needle substrates of pressure sensor. FIG. 43A, Lateral geometry and FIG. 43B, three dimensional geometry of Mg needle, also referred herein as a sharp-edged, substrates.

FIG. 44. Surgical process for injectable form of biodegradable sensors. A needle shaped sensor is positioned with a jig. Lowering the jig causes the sharp edge of the device to penetrate into the deep brain.

FIG. 45. Fully transient pressure sensor structure from a perspective (top panel) and side view (bottom panel).

FIG. 46. Transient pressure sensor calibration with mass-load, for a target application that is measuring brain pressure, with expected intracranial pressures of about 10 mmHg to 100 mmHg. Sensors may include Si on PLGA substrates with Si bottom substrate, and Si-NMs bonded on Si bottom structure.

FIGS. 47A-47D. Transient temperature sensor.

FIG. 48. Temperature and pressure sensors, Dissolution of Si in aCSF and In-vitro test of Si/PLGA sensor.

FIG. 49. Temperature and pressure sensors, Ex-vivo tests.

FIG. 50. Temperature and pressure sensors, In-vivo studies.

FIG. 51. Temperature and pressure sensors, FEM simulation of diaphragm structure, illustrating the 3 μm Si membrane is about 2 times more sensitive than 20 μm PLGA because flexural modulus is proportional to thickness⁻² and E¹.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of the invention.

“Bioresorbable” refers to a material that is susceptible to being chemically broken down into lower molecular weight chemical moieties by reagents and conditions that are naturally present in a biological environment. In an in-vivo application, the chemical moieties may be assimilated into human or animal tissue, or otherwise removed from the point of implantation. A bioresorbable material that is “substantially completely” resorbed is highly resorbed (e.g., 95% resorbed, or 98% resorbed, or 99% resorbed, or 99.9% resorbed, or 99.99% resorbed), but not completely (i.e., 100%) resorbed. “Bioresorption rate”, therefore, refers to the rate at which a bioresorbable material is broken down. The rate may be expressed in terms of a thickness of the material divided by the time required for the material to be broken down.

“Controlled bioresorption” refers to an arrangement of components and selection of materials so that the sensor operates for a desired length of time after implantation, after which continued bioresorption ensures no detectable portion of the sensor remains at the implantation site. For example, barrier layers and/or encapsulation layers may be utilized to temporarily protect certain components from exposure to the biological environment, including a biofluid that would otherwise start to hydrolyze the components or other biological material that would otherwise enzymatically or metabolically act on the components. Such controlled bioresorption ensures that complete bioresorption occurs after the desired operational lifetime.

“Operational lifetime” refers to a time period over which desired functionality of the sensor remains. This reflects that, before complete bioresorption, there is a time period where parameters may not be accurately measured, including due to partial degradation of one or more components of the sensor. The operational lifetime may be quantifiably defined in terms of providing an output within a user-defined tolerance level, such as an output that corresponds to a parameter magnitude that is no more than 20%, 15%, 10%, or 5% different from a true value. Accordingly, the operational lifetime may be selected between a range of days to months, such as 1 day to 4 months. Additional time beyond the operational lifetime may be required so that no detectable portion of sensor remains at the implantation site.

“No detectable portion” refers to the implanted sensor at the sensor site that is not detectable, including by the naked eye or by routine microscopic inspection, such as at a magnification power of 20× or more. The no detectable portion may also include that at the implantation sight there is no substantial indication of an immune response. This is a reflection that materials that are not readily observable may still initiate an immune response that can be quantified by any one or more assays known in the art. Such assays include detection of biomarkers related to an immune response, including immune cell migration and localization, or other biological marker indicative of an immune response change. The immune response change may be less than 10%, or less than 20%, or less than 25%, from a baseline value that is a no implantation condition in a human or animal. Accordingly, no detectable portion may accommodate a minor amount of material that may be detected using extraordinary measures, such as mass spectroscopy, but that is at such a low level, if any, that there is simply no effect on the biological tissue of such a low level. The no detectable portion outcome may be achieved by one or more processes, including breakdown into smaller chemical constituents that may be resorbed by the body and optionally subsequently excreted. In addition, the smaller chemical constituents may dissipate from the implant site and portions stored elsewhere in the body. The dissipation may be diffusive or convective in nature.

“Adjacent” refers to a position of a first component relative to a second component such that a physical effect arising from the first component is reliably transmitted to the second component. An example of such an adjacent position includes a pressure sensor strain gauge that is adjacent to a cavity. In this manner, deflection of the strain gauge arises from a pressure differential between fluid outside the cavity to the pressure in the cavity. The adjacent position ensures the strain gauge reliably senses this pressure difference. Similarly, adjacent may be described in more absolute terms, such as having at least a portion of the strain gauge the is positioned vertically over the cavity, including centered on one edge of the cavity and extending over the cavity. Adjacent may also encompass situations where there is an intervening component, such as a deformable layer that is positioned between the cavity and the stain sensor. Adjacent may also refer to a distance that overlaps or is within 50 μm, 10 μm or 1 μm of a pair of components.

“Electronic device” generally refers to a device incorporating a plurality of components, and includes wire circuits, integrated circuits, component arrays, biological and/or chemical sensors, and physical sensors (e.g., pressure, temperature, etc.).

“Sensing” refers to detecting the presence, absence, amount, magnitude or intensity of a physical and/or chemical property. Useful electronic device components for sensing include, but are not limited to piezoelectric elements, electrode elements, chemical or biological sensor elements, pH sensors, temperature sensors and capacitive sensors.

“Encapsulate” refers to the orientation of one structure such that it is at least partially, and in some cases completely, surrounded by one or more other structures. “Partially encapsulated” refers to the orientation of one structure such that it is partially surrounded by one or more other structures. “Completely encapsulated” refers to the orientation of one structure such that it is completely surrounded by one or more other structures. The invention includes implantable devices having partially or completely encapsulated inorganic semiconductor components and/or electrodes. An “encapsulating layer” may be employed to provide desired handling and protection characteristics, including so that the sensor is not adversely impacted during implantation. Particularly fragile or sensitive portions of the sensor may be protected such as by being covered and/or embedded, wholly or partially, by an encapsulation layer.

“Barrier layer” refers to a component spatially separating two or more other components or spatially separating a component from a structure, material or fluid external to the device. In one embodiment, a barrier layer encapsulates one or more components. In some embodiments, a barrier layer separates one or more components from an aqueous solution, a biological tissue or both. There may be distinct barrier and encapsulating layers. Alternatively, the barrier layer may also function as an encapsulating layer.

“Active circuit” and “active circuitry” refer to one or more components configured for performing a specific function. Useful active circuits include, but are not limited to, amplifier circuits, multiplexing circuits, current limiting circuits, integrated circuits, transistors and transistor arrays.

“Substrate” refers to a material, layer or other structure having a surface, such as a receiving surface, that is capable of supporting one or more components or electronic devices. A component that is “bonded” to the substrate refers to a component that is in physical contact with the substrate and unable to substantially move relative to the substrate surface to which it is bonded. Unbonded components or portions of a component, in contrast, are capable of substantial movement relative to the substrate.

“Biocompatible” refers to a material that does not elicit an immunological rejection or detrimental effect when it is disposed within an in-vivo biological environment. For example, a biological marker indicative of an immune response changes less than 10%, or less than 20%, or less than 25%, or less than 40%, or less than 50% from a baseline value when a biocompatible material is implanted into a human or animal.

“Bioinert” refers to a material that does not elicit an immune response from a human or animal when it is disposed within an in-vivo biological environment. For example, a biological marker indicative of an immune response remains substantially constant (plus or minus 5% of a baseline value) when a bioinert material is implanted into a human or animal.

“Polymer” refers to a macromolecule composed of repeating structural units connected by covalent chemical bonds or the polymerization product of one or more monomers, often characterized by a high molecular weight. The term polymer includes homopolymers, or polymers consisting essentially of a single repeating monomer subunit. The term polymer also includes copolymers, or polymers consisting essentially of two or more monomer subunits, such as random, block, alternating, segmented, grafted, tapered and other copolymers. Useful polymers include organic polymers or inorganic polymers that may be in amorphous, semi-amorphous, crystalline or partially crystalline states. Crosslinked polymers having linked monomer chains are particularly useful for some applications. Polymers useable in the methods, devices and components include, but are not limited to, plastics, elastomers, thermoplastic elastomers, elastoplastics, thermoplastics and acrylates. Exemplary polymers include, but are not limited to, acetal polymers, biodegradable polymers, cellulosic polymers, fluoropolymers, nylons, polyacrylonitrile polymers, polyamide-imide polymers, polyimides, polyarylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin or any combinations of these.

As used herein, semiconductor or metallic component refers to a portion of an electronic device used to sense or monitor a physical property or parameter of interest. Examples of components include layer(s) of material(s), such as nanomembranes, including layers arranged and patterned so as to provide a desired functionality. For example, monitoring or actuation of an electrical parameter may be accomplished by a component that is an electrode. Temperature or pressure may be accomplished by a piezoresistive component, with an electrical output that corresponds to the magnitude of the relevant parameter that is being monitored.

“Semiconductor” refers to any material that is an insulator at a very low temperature, but which has an appreciable electrical conductivity at a temperature of about 300 Kelvin. In the present description, use of the term semiconductor is intended to be consistent with use of this term in the art of microelectronics and electronic devices. Useful semiconductors include those comprising elemental semiconductors, such as silicon, germanium and diamond, and compound semiconductors, such as group IV compound semiconductors such as SiC and SiGe, group III-V semiconductors such as AlSb, AlAs, AlN, AlP, BN, BP, BAs, GaSb, GaAs, GaN, GaP, InSb, InAs, InN, and InP, group III-V ternary semiconductors alloys such as AlₓGa₁₋ₓAs, group II-VI semiconductors such as CsSe, CdS, CdTe, ZnO, ZnSe, ZnS, and ZnTe, group I-VII semiconductors such as CuCl, group IV-VI semiconductors such as PbS, PbTe, and SnS, layer semiconductors such as PbI₂, MoS₂, and GaSe, oxide semiconductors such as CuO and Cu₂O. The term semiconductor includes intrinsic semiconductors and extrinsic semiconductors that are doped with one or more selected materials, including semiconductors having p-type doping materials and n-type doping materials, to provide beneficial electronic properties useful for a given application or device. The term semiconductor includes composite materials comprising a mixture of semiconductors and/or dopants. Specific semiconductor materials useful for some embodiments include, but are not limited to, Si, Ge, Se, diamond, fullerenes, SiC, SiGe, SiO, SiO₂, SiN, AlSb, AlAs, AlIn, AlN, AlP, AlS, BN, BP, BAs, As₂S₃, GaSb, GaAs, GaN, GaP, GaSe, InSb, InAs, InN, InP, CsSe, CdS, CdSe, CdTe, Cd₃P₂, Cd₃As₂, Cd₃Sb₂, ZnO, ZnSe, ZnS, ZnTe, Zn₃P₂, Zn₃As₂, Zn₃Sb₂, ZnSiP₂, CuCl, PbS, PbSe, PbTe, FeO, FeS₂, NiO, EuO, EuS, PtSi, TlBr, CrBr₃, SnS, SnTe, PbI₂, MoS₂, GaSe, CuO, Cu₂O, HgS, HgSe, HgTe, HgI₂, MgS, MgSe, MgTe, CaS, CaSe, SrS, SrTe, BaS, BaSe, BaTe, SnO₂, TiO, TiO₂, Bi₂S₃, Bi₂O₃, Bi₂Te₃, BiI₃, UO₂, UO₃, AgGaS₂, PbMnTe, BaTiO₃, SrTiO₃, LiNbO₃, La₂CuO₄, La_{0.7}Ca_{0.3}MnO₃, CdZnTe, CdMnTe, CuInSe₂, copper indium gallium selenide (CIGS), HgCdTe, HgZnTe, HgZnSe, PbSnTe, Tl₂SnTe₅, Tl₂GeTe₅, AlGaAs, AlGaN, AlGaP, AlInAs, AlInSb, AlInP, AlInAsP, AlGaAsN, GaAsP, GaAsN, GaMnAs, GaAsSbN, GaInAs, GaInP, AlGaAsSb, AlGaAsP, AlGaInP, GaInAsP, InGaAs, InGaP, InGaN, InAsSb, InGaSb, InMnAs, InGaAsP, InGaAsN, InAlAsN, GaInNAsSb, GaInAsSbP, and any combination of these. Porous silicon semiconductor materials are useful for aspects described herein. Impurities of semiconductor materials are atoms, elements, ions and/or molecules other than the semiconductor material(s) themselves or any dopants provided to the semiconductor material.

A “semiconductor component” broadly refers to any semiconductor material, composition or structure, and expressly includes high quality single crystalline and polycrystalline semiconductors, semiconductor materials fabricated via high temperature processing, doped semiconductor materials, inorganic semiconductors, and composite semiconductor materials.

A “component” is used broadly to refer to an individual part of a device. An “interconnect” is one example of a component, and refers to an electrically conducting structure capable of establishing an electrical connection with another component or between components. In particular, an interconnect may establish electrical contact between components that are separate. Depending on the desired device specifications, operation, and application, an interconnect is made from a suitable material. Suitable conductive materials include metals, alloys thereof, and semiconductors.

Other components include, but are not limited to, resistors, capacitors, diodes, thin film transistors (TFTs), transistors, electrodes, integrated circuits, circuit elements, control elements, microprocessors, transducers, islands, bridges and combinations thereof. Components may be connected to one or more contact pads as known in the art, such as by metal evaporation, wire bonding, and application of solids or conductive pastes, for example.

Provided herein are sensors that can measure multiple physical parameters simultaneously, including temperature and pressure within a compartment, such as the brain, for a period of one week. The device is constructed from biocompatible and bioresorbable materials and dissolves and is resorbed into the body after providing high-fidelity measurements for an operational lifetime, including a period of hours, one day, one week, or more. For example, a pressure and temperature sensor device can be used for any compartment within the body, including the brain, legs, arms, chest, organs, and others. This sensor can send measurement data through a wired or wireless connection to an external interrogator which can collect the data.

In this manner, a method of diagnosing and monitoring pressure within a body compartment in the least invasive way possible is provided. The method is significantly less invasive than traditional methods because the sensors provided herein require only one surgery for implantation. Once in place, the sensor can monitor brain pressure, for example, and wirelessly transmit critical data and then be left to resorb into the brain without the need for a second surgery to remove the device. One specific application is for the treatment of traumatic brain injury.

Traumatic brain injury (TBI) remains a major cause of death and disability worldwide. In the United States alone, more than 53,000 individuals die annually because of TBI, contributing to 30.5% of all injury-related deaths. Among those who die from TBI, the majority die because of uncontrolled rise of intracranial pressure (ICP), mostly within the first 48 hours of injury. After a severe TBI, efforts are focused on prevention of further damage through intensive monitoring and prompt intervention. Approximately 17.6% adult patients and 55% of pediatric patients undergo ICP monitoring post head injury. The median duration of monitoring is 7 days. Current ICP monitoring devices have a device complication rate of 4.9% not including infection and hemorrhage. The most frequent complication, aside from infection, was disconnection of the transducer. This complication, only rarely reported, generally occurs during patient transport and care. A wireless dissolvable ICP monitor is a major advance avoiding these complications.

In the case of an intracranial pressure monitor, the device can be implanted into the brain and pressure monitored, including for 1 week or more. The device does not have to be removed from the brain, thereby reducing complications associated with infection and precluding the patient from needing to go into surgery a second time. Additionally, the device can transmit data wirelessly to the patient and doctor and enable the patient to return home from the hospital while continuing to monitor pressure within the brain. An automatic alert may be provided to the medical professional and/or patient should any readings fall out of a tolerance range.

### Example 1

### Implantable and Bioresorbable Sensors

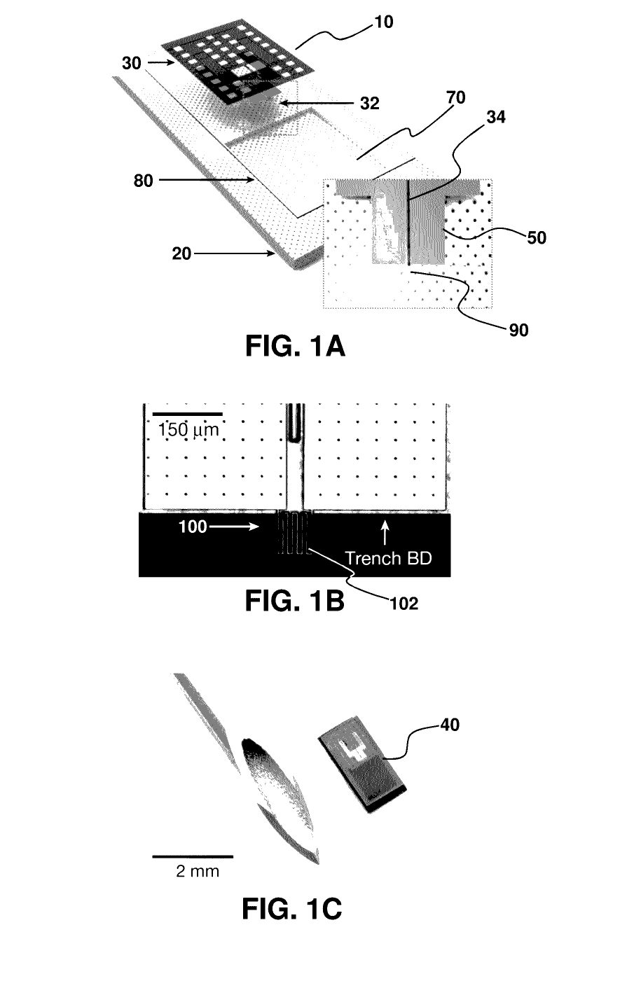

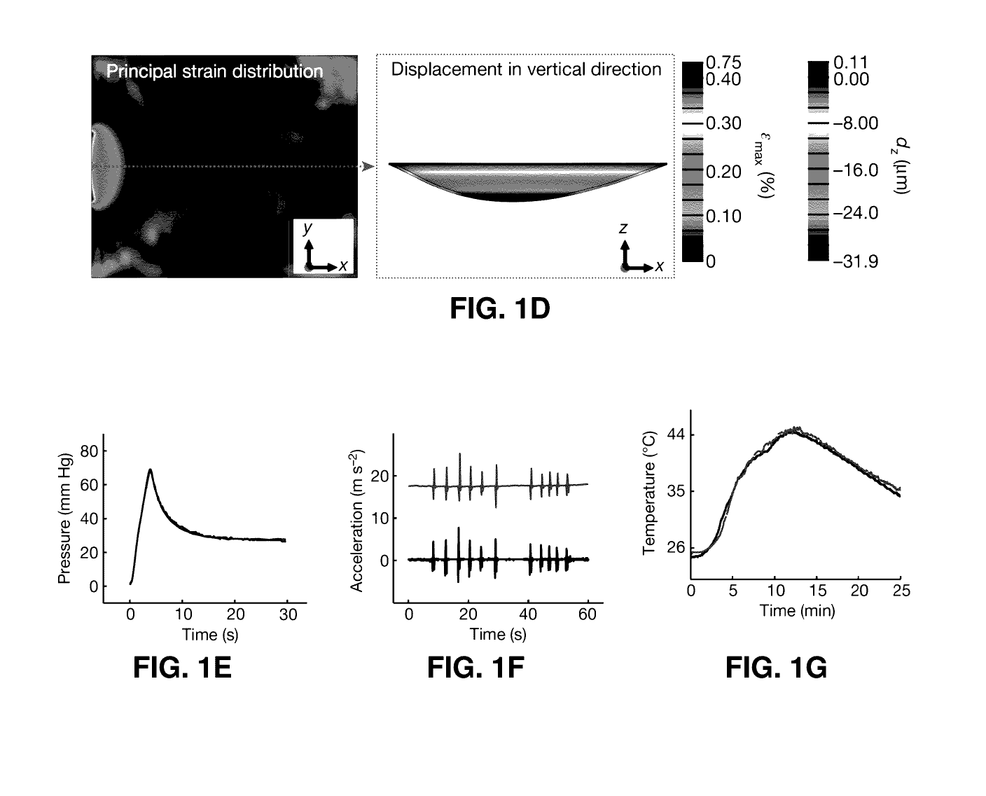

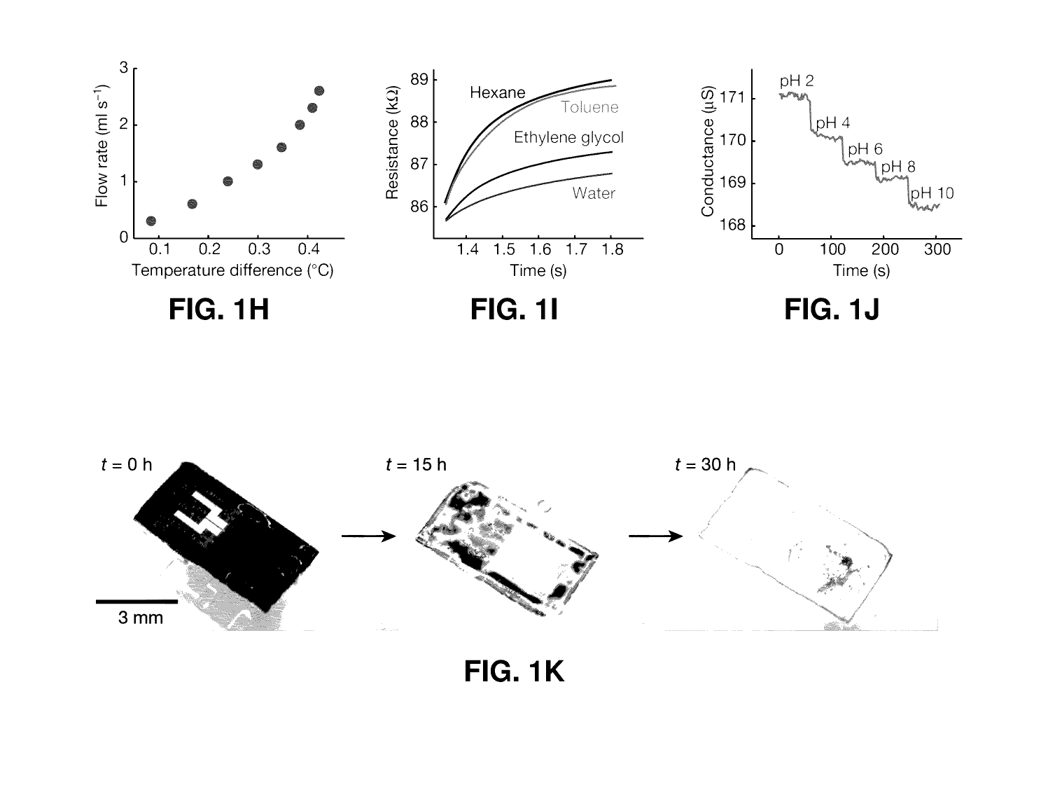

Referring to FIGS. 1A-1C, an implantable and bioresorbable sensor 10 has a substrate 20 that supports an electronic device 30. The electronic device may have semiconductor 32 and/or metallic components 34, in this case illustrated as Si-nanomembrane (Si-NM) having a distal piezoresistive end 100 with a serpentine geometry 102 and positioned adjacent to a cavity 70 disposed in the substrate 20. A barrier layer or an encapsulating layer 40 may be used to assist in achieved a desired controlled operational lifetime. The electronic device may form a sensor 50, including a pressure sensor having a cavity 70 covered by a deformable layer 80, wherein the Si-NM forms a strain gauge 90 that is positioned in an adjacent orientation relative to the cavity. In this example, adjacent refers to at least a portion of the distal end of the Si-NM that extends over the cavity 70 so that any deformation of the deformable layer 80 results in strain generation in the strain gauge 90 (see, e.g., FIG. 1D).

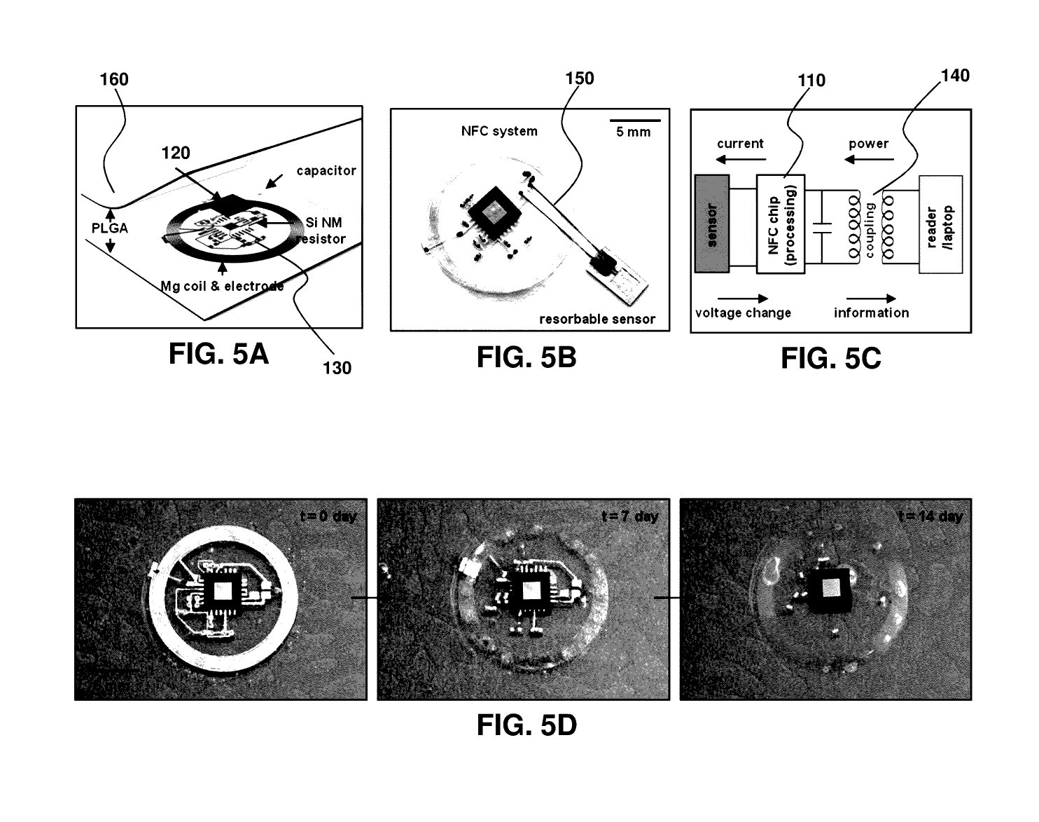

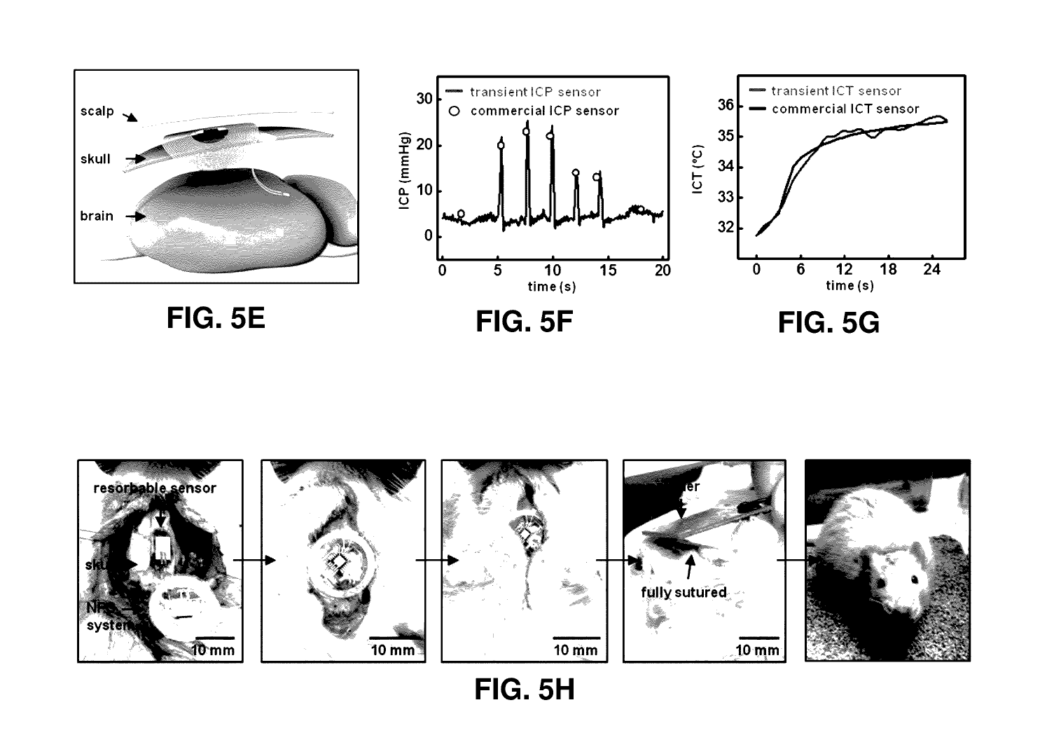

The sensor may be operably connected to a wireless data-transmitter 110, including as part of a NFC chip 120 (see, e.g., FIGS. 5A-5C). Referring to FIGS. 5A-5C, the electronic device may comprise an electronic circuit 130 having any number of electrical components, including electrical interconnects, resistors, capacitors (FIG. 5A) and that electrically connect the chip to the semiconductor and/or metallic components of the electronic device. An inductive coil or radiofrequency receiver 140, may be used to power the sensor and receive data from the sensor (FIG. 5C). FIG. 5B illustrates bioresorbable wire 150 that can electrically connect the sensor's electronic device to a wireless transmitter. Also illustrated, is an encapsulating layer 160, indicated as PLGA.

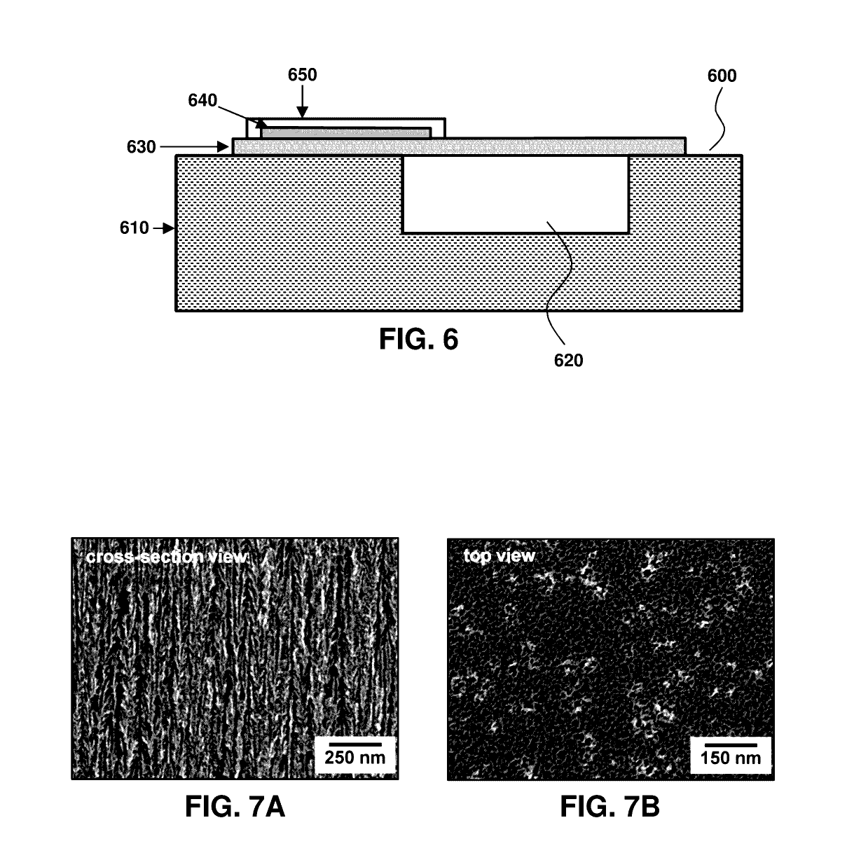

FIG. 6 is a side view of an implantable and bioresorbable sensor that has at least a pressure-sensing functionality. The implantable and bioresorbable pressure sensor 600 has a substrate 610 (e.g., nanoporous Si (np-Si) or Mg foil), air cavity 620, deformable layer 630 (e.g., PLGA) that forms an air-tight seal with cavity 620, electronic device 640 (e.g., Si-NM), barrier layer 650 (e.g., SiO₂). In this manner, a pressure difference associated with a pressure in a fluid outside the air cavity and exerted on a top surface of the deformable layer 630 relative to the pressure in the air cavity exerted on the bottom surface of the deformable layer 630 is detected by a piezoresistive element of electronic device 640 that is adjacent to the cavity. As desired, an encapsulation layer (not shown) may surround all or part of the sensor 600.

Each of these illustrated elements are configured to independently dissolve in the body after a suitable operational lifetime. While the dissolution of each constituent may be independent, with metals, semiconductors, polymers, porosity, accessibility, composition, each influencing dissolution and subsequent bioresorption characteristics, configuration and positioning of layers may be used to delay dissolution. For example, additional layers may be positioned around otherwise exposed layers to delay dissolution. Similarly, encapsulation layers may be used around the sensor to extend sensor lifetime. For applications where short-term monitoring is desired, no or an extremely thin encapsulation layer may be used. In contrast, for monitoring over a long term of weeks or months, a highly resistant to dissolution encapsulation layer may be used.

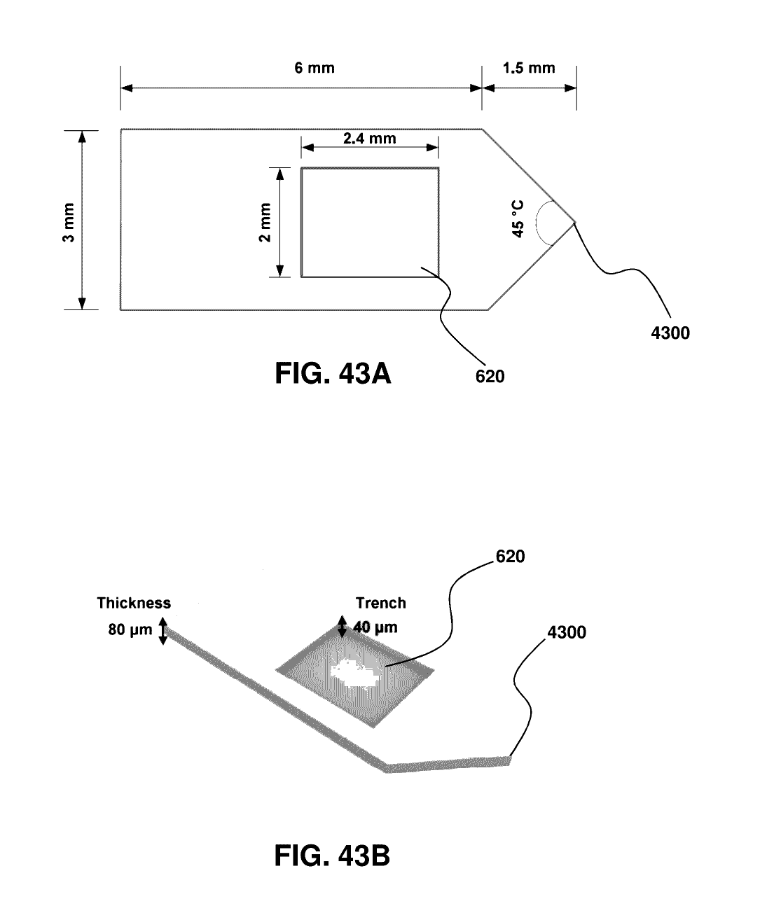

To facilitate implantation and minimize tissue damage, any of the sensors may have a sharp-edged leading tip 4300 (see, e.g., FIG. 43A-43B) having an entry angle, such as between 30° and 60°, and illustrated in FIG. 43A as 45°. The cavity 620 in the substrate may be defined by a length, width and depth, illustrated in this example as 2.4 mm×2 mm×40 μm. Alternatively, ratios can be used to define the dimensions relative to the dimensions of the substrate (6 mm+1.5 mm) long, 3 mm wide, 80 μm thick. Exemplary ratios include a cavity depth that is between 10% and 80% of the substrate thickness, cavity length that is between 10% and 80% of the total substrate length, cavity width that is between 10% and 80% of maximum substrate width, and any subranges thereof.

### Example 2

### Bioresorbable Silicon Electronic Sensors for the Brain

Many procedures in modern clinical medicine rely on the use of electronic implants in treating conditions that range from acute coronary events to traumatic injury. However, standard permanent electronic hardware acts as a nidus for infection: bacteria form biofilms along percutaneous wires, or seed haematogenously, with the potential to migrate within the body and to provoke immune-mediated pathological tissue reactions. The associated surgical retrieval procedures, meanwhile, subject patients to the distress associated with re-operation and expose them to additional complications. Provided herein are materials, device architectures, integration strategies, and in vivo demonstrations of implantable, multifunctional silicon sensors for the brain, for which all of the constituent materials naturally resorb via hydrolysis and/or metabolic action, eliminating the need for extraction. Continuous monitoring of intracranial pressure and temperature illustrates functionality essential to the treatment of traumatic brain injury; the measurement performance of the resorbable devices provided herein compares favorably with that of non-resorbable clinical standards. In this example, insulated transdermal wires connect to an externally mounted, miniaturized wireless potentiostat for data transmission. In a separate set-up, a sensor is connected to an implanted (but only partially resorbable) data-communication system, establishing that the devices described herein do not require any transcutaneous wiring. The devices can be adapted to sense any of a range of physical parameters, including fluid flow, motion, pH or thermal characteristics, in formats that are compatible with any number of body cavities, including abdominal and extremity compartments, as well as the deep brain, reflecting that the sensors may meet many needs in clinical medicine.

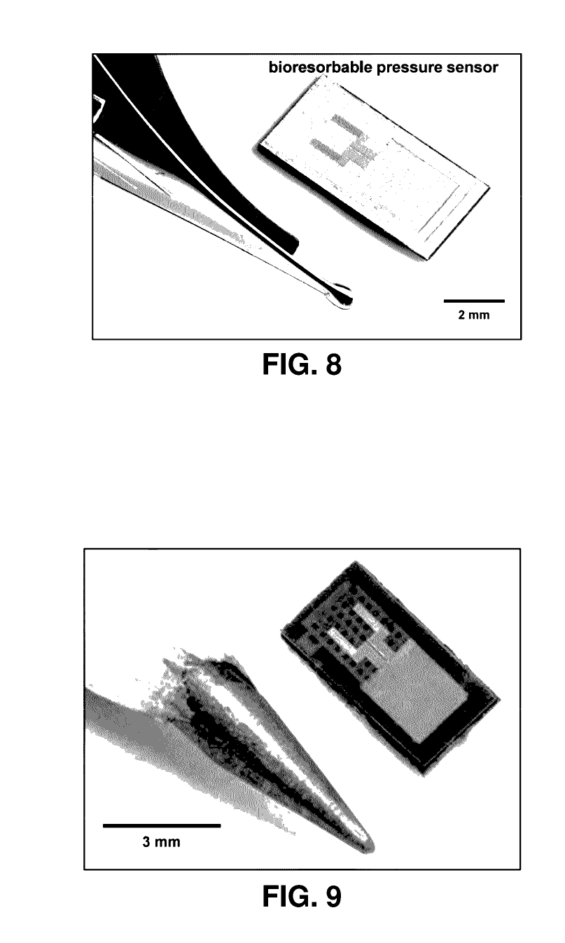

FIG. 1A and FIG. 6 show a bioresorbable pressure sensor with a magnified illustration of the active region and its cross-sectional side view. The construction involves a membrane of poly(lactic-co-glycolic acid) (PLGA, with a thickness of 30 μm), sealed against a supporting substrate of nanoporous silicon (60-80 μm thick; 71% porosity) or magnesium foil (60-80 μm thick; see FIGS. 7, 8). The supporting substrate has a square structure of relief (with a depth of 30-40 μm) etched onto its surface. The associated air cavity allows the membrane to deflect in response to pressure in the fluid surroundings. A silicon nanomembrane in a serpentine geometry serves as a piezoresistive element that rests on the surface of the membrane near one of the edges of the cavity, where deflection-induced strains are largest (FIG. 1B). The resistance of this sensing element increases monotonically in a linear fashion across the full range of pressures that are relevant to intracranial monitoring (that is, 0-70 mm Hg). An overcoat of silicon oxide (SiO₂, about 100 nm thick) provides electrical passivation and a barrier against biofluids. FIG. 1C and FIG. 9 show photographs of two representative devices of different dimensions to illustrate the scalability of fabrication; the total sizes and weights are 1 mm×2 mm×0.08 mm (trench size: 0.67 mm×0.8 mm×0.03 mm) and about 0.4 mg; and 3 mm×6 mm×0.11 mm (trench size: 2 mm×2.4 mm×0.04 mm) and roughly 1 mg, respectively.

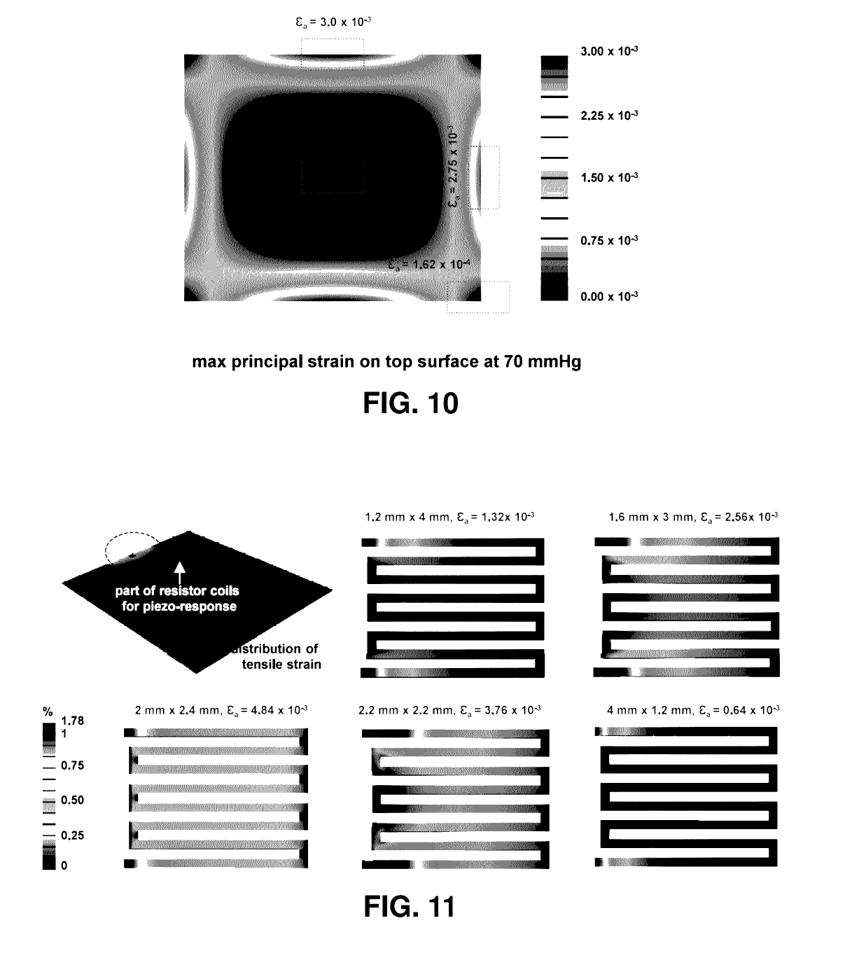

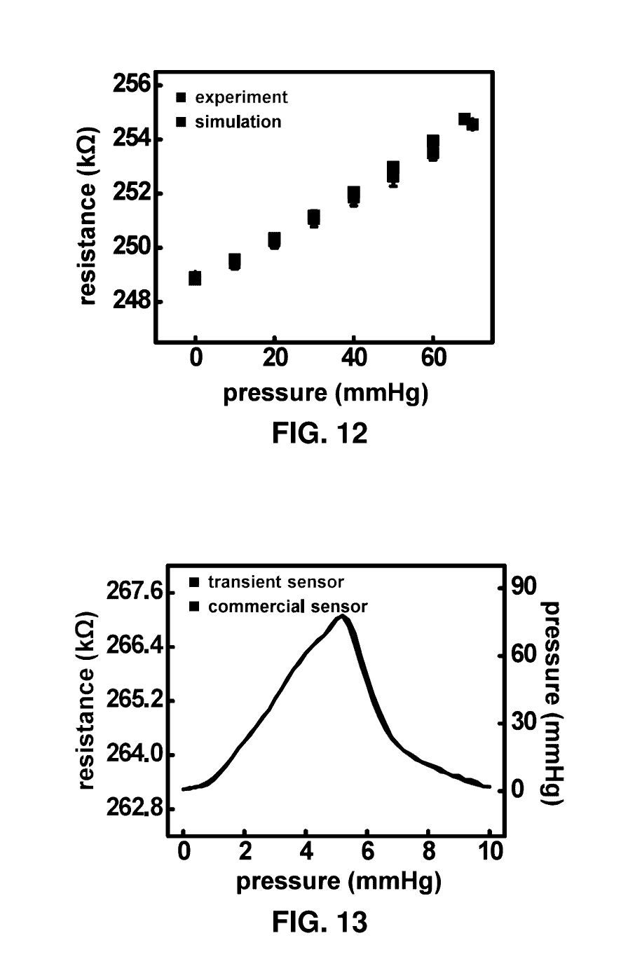

The mechanics of the system can be captured quantitatively by three-dimensional finite element analysis (FEA). Distributions of principal strains and vertical displacements evaluated at an external pressure of 50 mm Hg appear in FIG. 1D. The maximum strain for any applied pressure over the range of interest occurs at the midpoint of the left (and right) edge of the trench, thus motivating this choice of location for the silicon-nanomembrane piezoresistive element (see, e.g., FIGS. 10 and 11 for details). The calibration between pressure and resistance is linear, with a slope of 83 Ωmm Hg⁻¹, consistent with modelling results and a gauge factor of about 30, which lies within a range of expected values for monocrystalline silicon (FIG. 12).

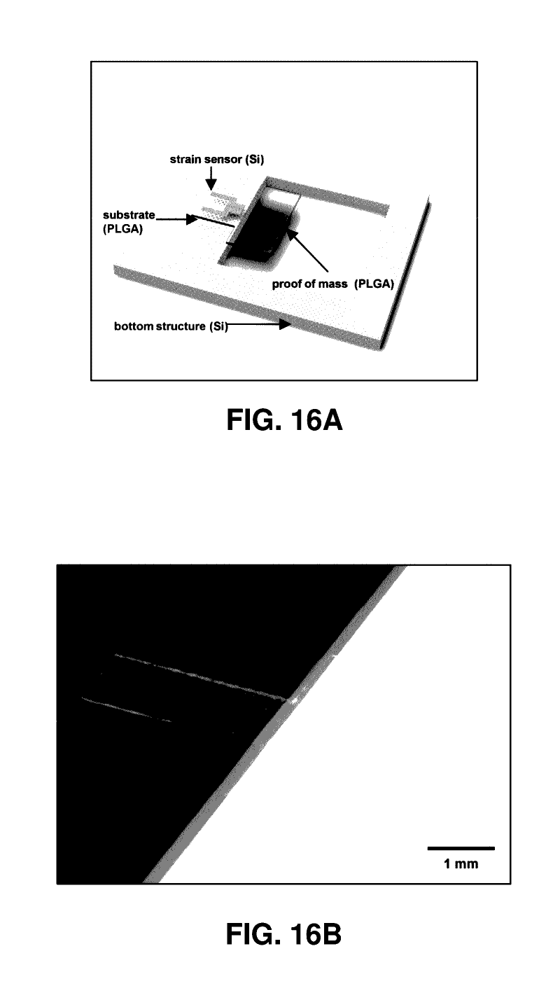

Evaluations in set-ups that resemble the intracranial cavity reveal measured pressure responses that agree quantitatively with those of clinical-standard, non-bioresorbable sensors (FIG. 1E and FIGS. 13-15). With various simple modifications, this same platform can be used for precision measurement of other parameters of interest in biomedicine and clinical care. Examples include: motion sensors built with a cantilevered test mass of PLGA (that is, a single-axis accelerometer, FIG. 1F); temperature sensors that exploit the temperature-dependent resistance of silicon-nanomembrane elements set apart from the cavity structure (FIG. 1G); flow sensors in which the silicon nanomembranes serve simultaneously as heating elements and temperature sensors (FIG. 1H); thermal conductivity/diffusivity sensors that exploit related concepts (FIG. 1I); and pH sensors that rely on electrostatic gating of transport through the silicon nanomembrane (FIG. 1J). In addition, chemically functionalizing the surface of the silicon of this last device provides a route to biomolecular sensing, using schemes similar to those in conventional silicon biosensors, including for detection or quantification of any number of relevant biological materials, including glucose, lactose and oxygen. The fabrication methods and operating principles for each of the modalities in FIGS. 1F-1J are further discussed below and in FIGS. 16-20.

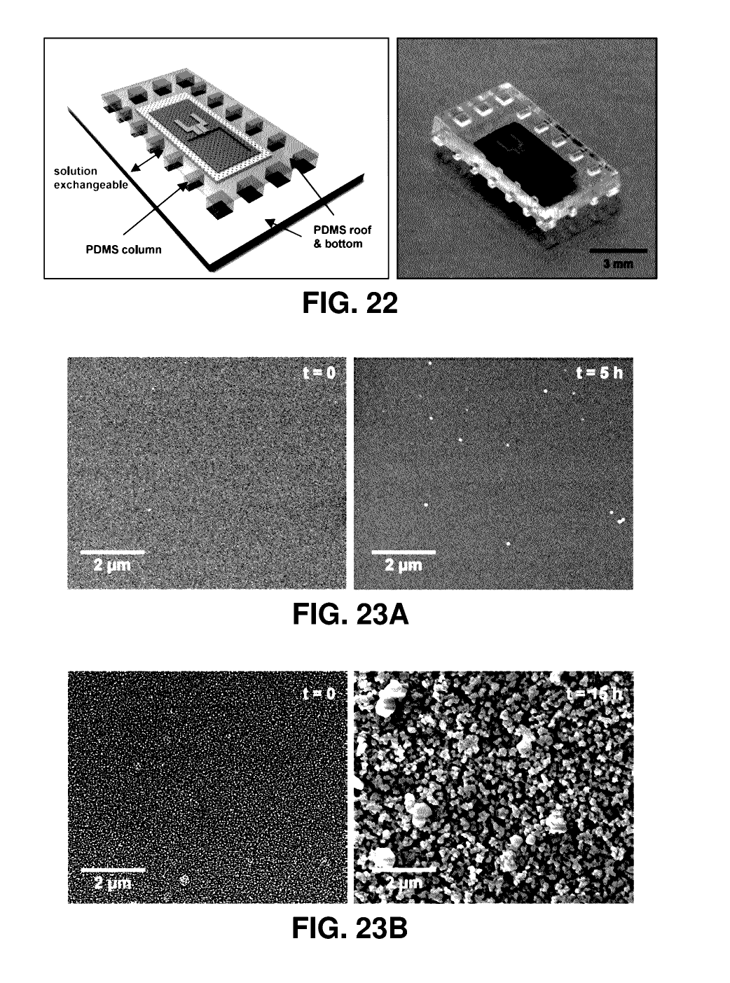

The uniqueness of these devices is their ability to dissolve completely into biocompatible end products when immersed in aqueous solutions, including biofluids such as cerebrospinal fluid (CSF). Hydrolysis of the silicon nanomembranes, the layers of SiO₂, the thin wafers of nanoporous silicon and the magnesium foils causes loss of material at rates of about 23 nm day⁻¹, 8 nm day⁻¹, 9 μm day⁻¹ and 4 μm day⁻¹, respectively, in artificial CSF (ACSF) at physiological temperature (37° C.) (FIG. 21). Separate studies indicate that PLGA (75:25 (lactide:glycolide) composition) dissolves in biofluids within four to five weeks. To illustrate the various stages of dissolution of a completed system, FIG. 1K shows a sequence of images of a bioresorbable pressure sensor inserted into a transparent chamber designed for accelerated testing (polydimethylsiloxane (PDMS) enclosure filled with buffer solution at pH 12 and room temperature), in which fluid exchange can occur through an array of openings around the perimeter (FIG. 22). FIG. 23A-23B presents images of nanoporous silicon and silicon nanomembranes observed by scanning electron microscopy at various stages of hydrolysis. The silicon nanomembrane dissolves uniformly, without fracture. By comparison, nanoporous silicon dissolves less uniformly, with a tendency to form fragments. Here, the silicon-nanomembrane and SiO₂ components dissolve first, within 15 hours, followed by the nanoporous silicon, which disappears within 30 hours. In all cases, the dissolution kinetics depends strongly on the materials and the composition of the surrounding solution. Table 1 summarizes the hydrolysis mechanisms and dissolution rates of these materials in a representative solution. As described below, the encapsulation material and its thickness define the operational lifetimes of the sensor(s).

TABLE 1
Hydrolysis mechanisms and dissolution rates of key materials in this
study (ACSF and PBS measured at 37° C., DI at room temperature)

| | Dissolution rate (nm/day) | | | |
|---|---|---|---|---|
| Materials | ACSF | PBS^{12, 21} | DI²² | Hydrolysis mechanism |
| Si NM | 2.3 × 10¹ | 0.5 × 10¹ | | Si + 4H₂O → Si(OH)₄ + 2H₂ |
| np-Si | 9.0 × 10³ | — | — | |
| SiO₂ | 0.8 × 10¹ | 1.4 × 10¹ | | SiO₂ + 2H₂O → Si(OH)₄ |
| Mg | 4.0 × 10³ | — | 1.7 × 10³ | MGO + H₂O → Mg(OH)₂ |
| Mo | — | 2.0 × 10¹ | 0.7 × 10¹ | 2Mo + 2H₂O + 3O₂ → 2H₂MoO₄ |

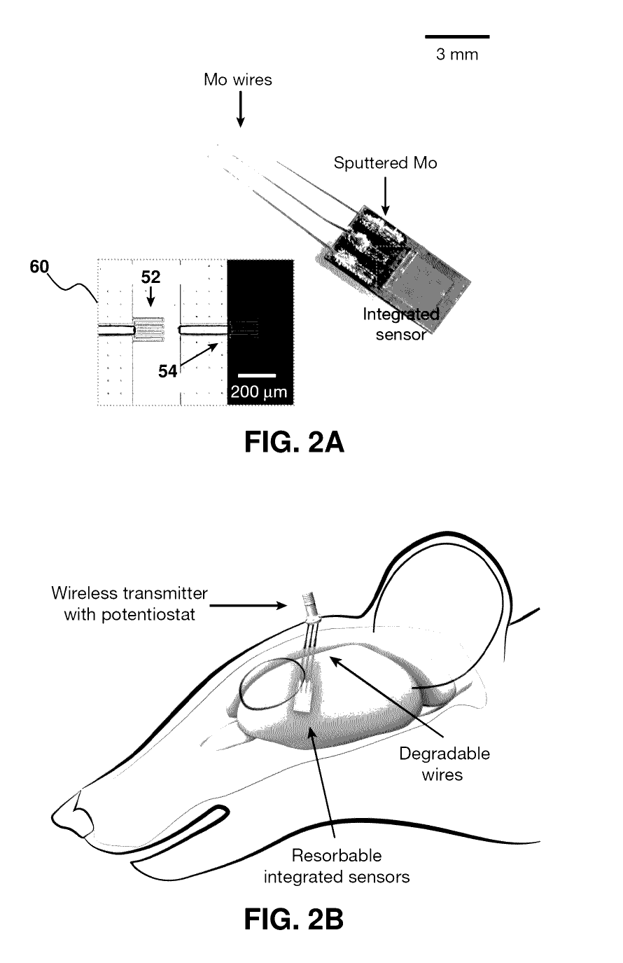

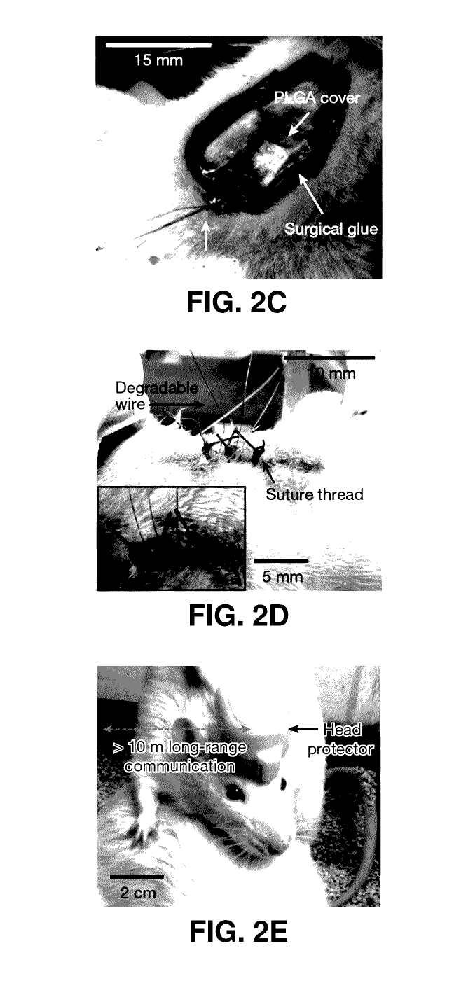

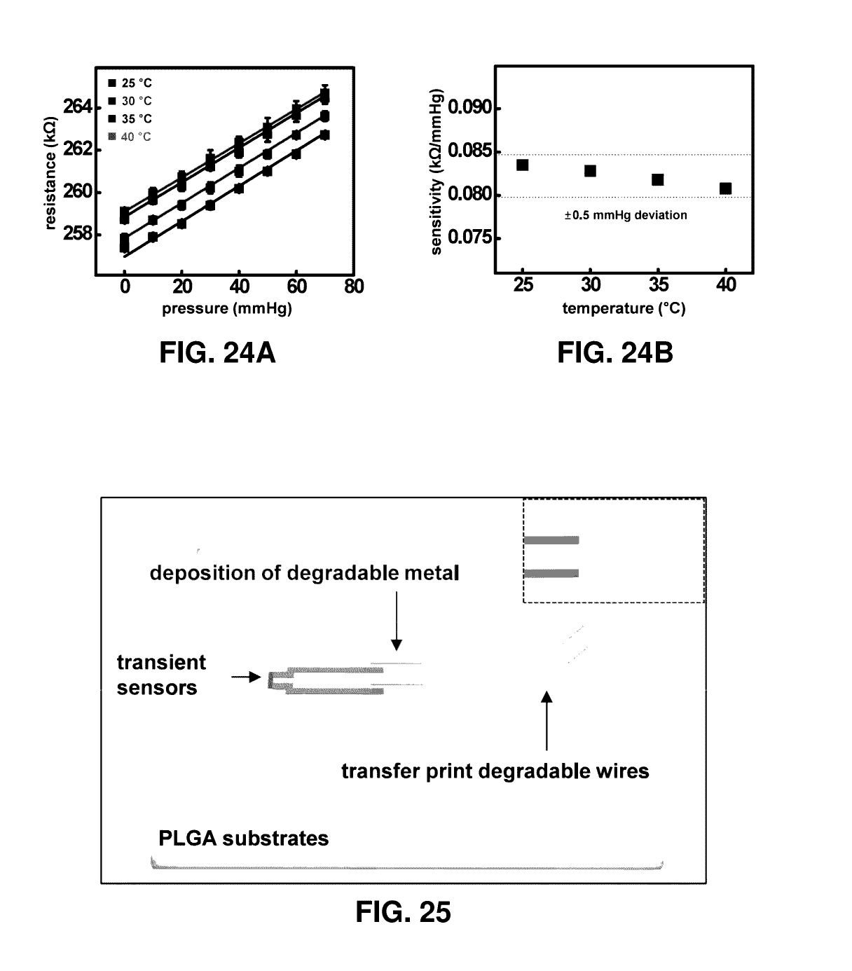

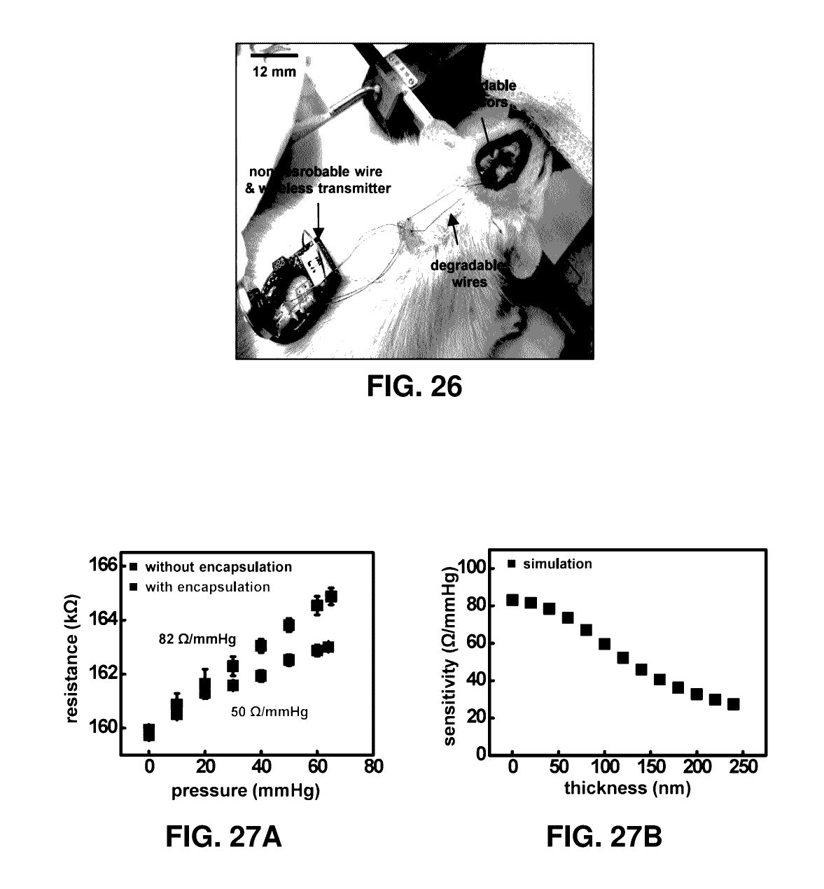

FIGS. 2A-2E illustrate a strategy for using these types of bioresorbable systems for wireless pressure and temperature monitoring in the intracranial space of rats. This configuration is referred herein as being “multi-functional” as at least two different physical parameters are measured, temperature with temperature sensor 52 and pressure with pressure sensor 54. FIG. 2A shows a photograph of a device like the one in FIG. 1C, but configured to allow simultaneous sensing of both pressure and temperature. Similarly, any other physical parameters may be measured, constrained only by the ability to reliably locate distinct sensors in distinct locations on the substrate so as to avoid interference and cross-talk. The measured temperature can also be used to calibrate against parasitic effects of this parameter on the pressure determination (see below and FIG. 24A-24B). Biodegradable molybdenum wires (10 μm thick) serve as an interface to wireless communication systems. Pressing the interconnect wires (molybdenum, 10 μm, or magnesium, 50 μm thick) against the PLGA at elevated temperatures (65° C.) embeds them near the surface but leaves the top regions exposed, thereby allowing for deposition of biodegradable metals (molybdenum, thickness 2 μm) to form electrical contact pads through stencil masks (made from the polyimide Kapton, 12.5 μm thick; FIG. 25). Metal wires and silicon nanomembranes are fully embedded in PLGA; the deposited molybdenum forms stable interconnects between them. Encapsulation with a bioresorbable polymer (polyanhydride, discussed in more detail below) enhances system robustness by reducing the stress concentrations at the interconnections. Narrow strips of PLGA laminated onto the front and back sides of the wires along their entire lengths act as electrical insulation. These insulated wires connect to an externally mounted, miniaturized wireless potentiostat for transmission of data thorough transdermal wiring. FIG. 2B provides a diagram of such a system in the intracranial space of a rat model. The sensor subsystem connects via molybdenum wires to the wireless module, which is mounted on the top of the skull. FIGS. 2C-2E summarize the surgical process. A PLGA sheet (about 80 μm thick) and a dissolvable surgical glue (FIG. 2C) seal the craniectomy defect to close the intracranial cavity. Conventional sutures hold the surgical site closed, in a standard process that retains points at which the dissolvable wires emerge from the skin to allow electrical connection (FIG. 2D). These wires have dimensions comparable to those of the surgical threads, and therefore pose little additional risk. FIG. 2E shows a healthy, freely moving rat with a complete system. FIG. 26 presents images of the connections.

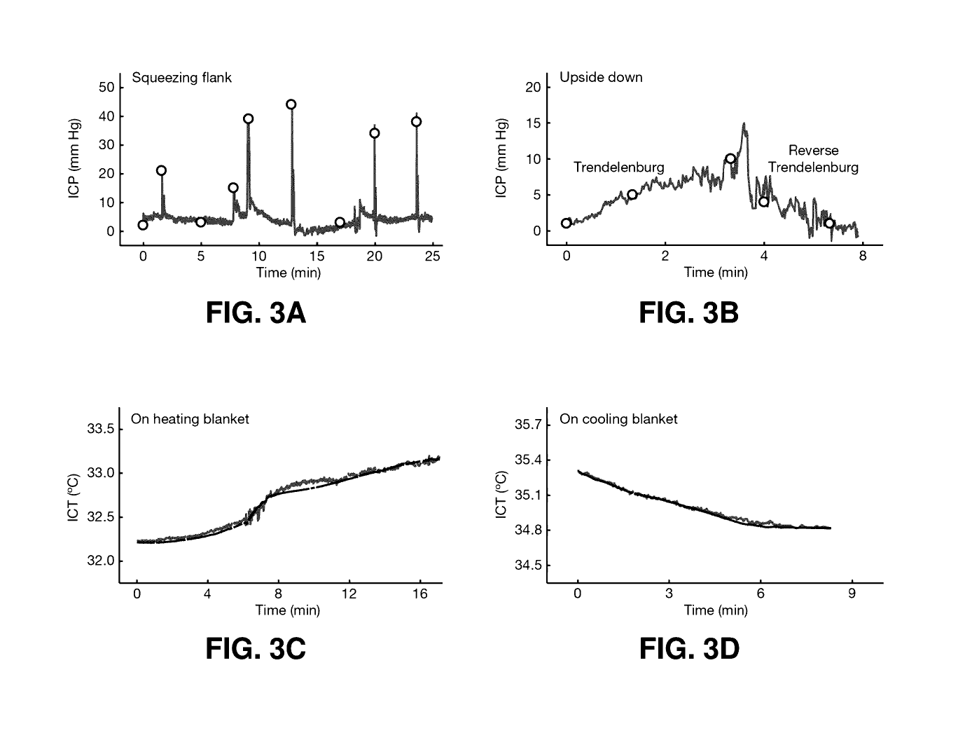

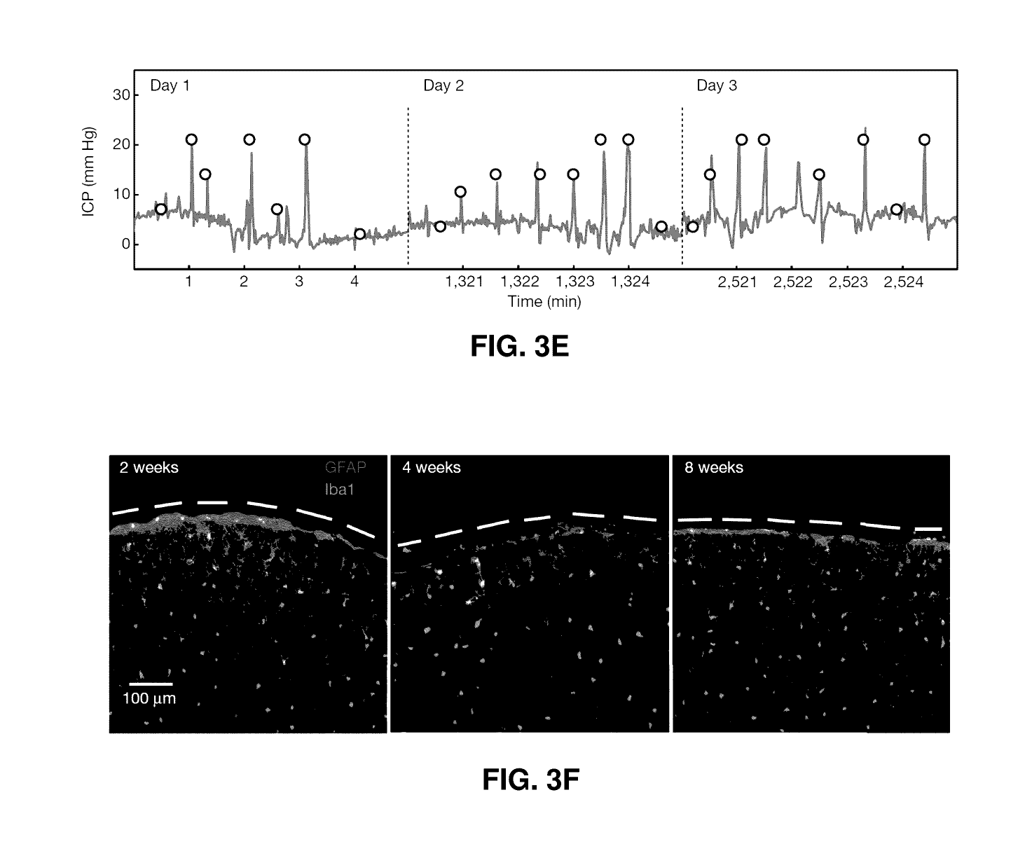

FIGS. 3A-3F summarize the results of a comprehensive set of wireless measurements of intracranial pressure (ICP) and intracranial temperature (ICT), recorded in rats with percutaneous wired systems. The ICP traces reveal features that correspond to periodic manual abdominal compression activated by the Valsalva maneuver, which yields rapid increases or decreases in the blood pressure in the intracranial space (FIG. 3A). Gentle changes in the rat's position—that is, Trendelenburg (30° head-down position) and reverse Trendelenburg (30° head-up position)—produce gradual increases and decreases in ICP, respectively (FIG. 3B), as would be expected because of the corresponding accumulation and depletion of blood in the brain. The pressure values compare well with those determined using a clinical-standard, wired ICP sensor implanted in the same region of the same animal. The wireless, bioresorbable ICT sensors perform to levels of accuracy similar to those of commercial sensors: FIGS. 3C and 3D show comparative data collected by modulating the cranial temperature with a heating or cooling blanket placed beneath the animal.

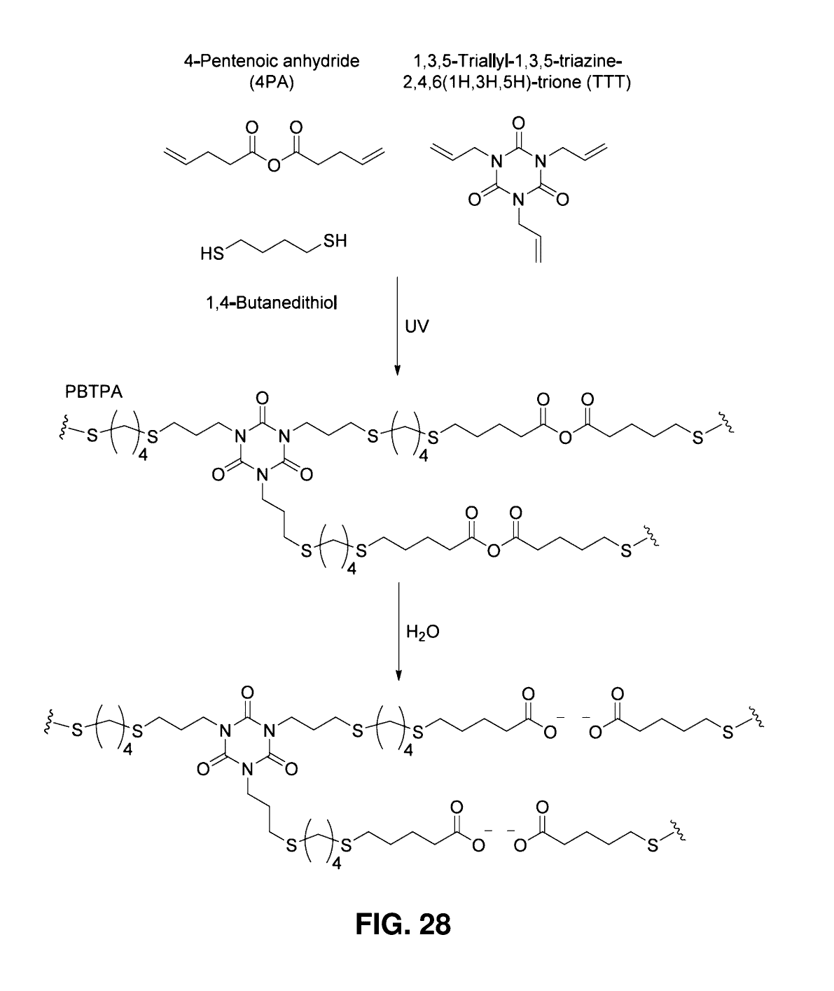

The operational lifetimes of the devices are defined by dissolution of the encapsulation layers and the permeation of fluids through them. In vitro experiments using a bioresorbable pressure sensor encapsulated with a film of a specially synthesized polyanhydride (about 120 μm thick; FIG. 27A-27B) show the expected performance and accurate readings with an appropriately modified calibration factor (50 Ωmm Hg⁻¹). The slow dissolution rate of the polyanhydride (about 1.3 μm day⁻¹)—together with the modest change in sensitivity that occurs depending on the thickness of this material (about 0.34 Ωmm Hg⁻¹ μm⁻¹)—leads to a loss of accuracy of only a few percent when operated over several days. This error falls within standards defined by the Association for the Advancement of Medical Instrumentation (AAMI) for pressure monitoring, that is, ±2 mm Hg (from 0 to 20 mm Hg) and ±10% (from 20 to 100 mm Hg). (below and FIGS. 28-30B present information on the synthesis/hydrolysis chemistry, dissolution kinetics, water permeability, and biocompatibility of the polyanhydride.)

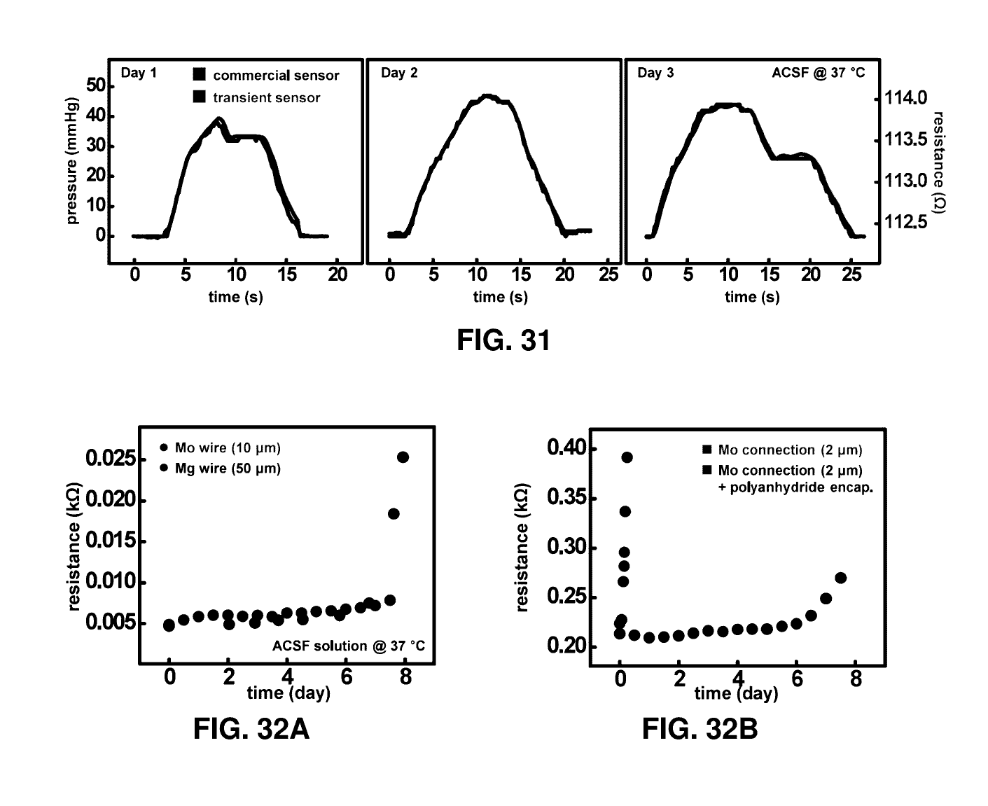

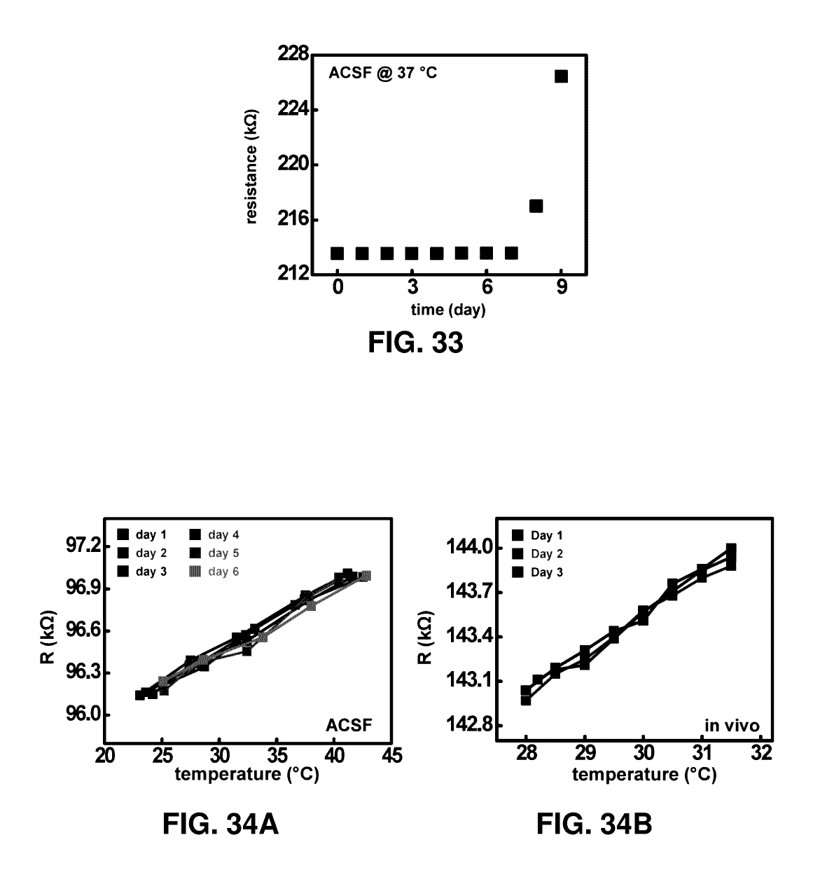

Stable, continuous operation is possible for up to three days (FIG. 31). Beyond this period, water tends to pass through the polyanhydride and PLGA into the electrically active regions of the device and the air cavity. The resistance remains relatively constant for seven days, and then begins to increase markedly, mainly because of dissolution of the molybdenum wires and interconnection metal (FIGS. 32 and 33). FIG. 3E illustrates in vivo operation for three days without notable degradation in absolute accuracy or sensitivity, as benchmarked against a standard, non-resorbable wired sensor. FIG. 34A-34B shows similar data from the temperature sensor, where the absence of an air cavity affords enhanced stability, and accurate measurements for six days of operation. These timeframes are relevant for clinical use: ICP and ICT are typically monitored continuously for several days after traumatic brain injury. The chemistry, thickness and composition of the encapsulating layers can be selected to extend the functional lifetimes.

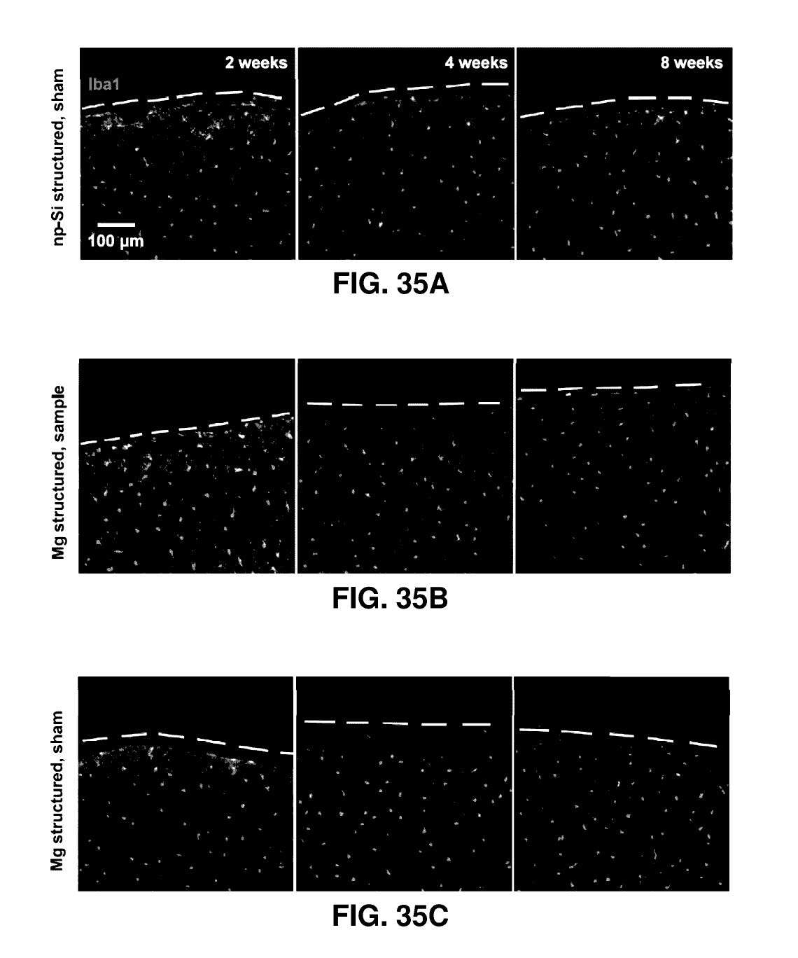

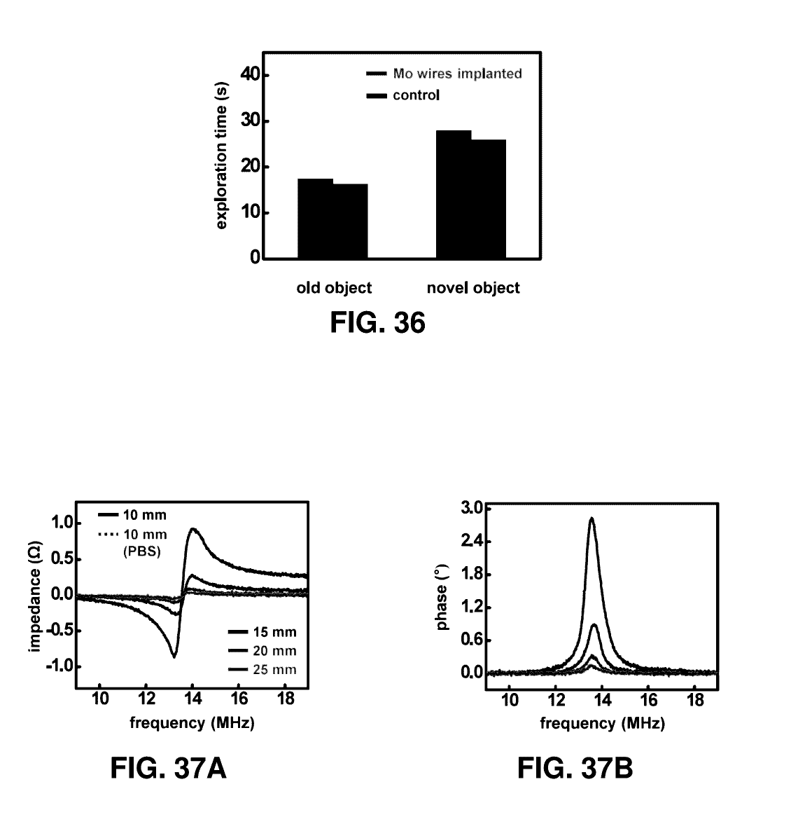

Biocompatibility of the devices through all stages of their life cycle is essential. Comprehensive studies of the immunohistochemistry of brain tissues at several times after implantation (two, four and eight weeks) demonstrate that the sensors and the by-products of their dissolution in the intracranial space are biocompatible. Representative confocal fluorescence images (see FIG. 3F for nanoporous silicon and FIG. 35A-35C for magnesium foil) indicate no overt reaction of brain glial cells to the sensor, and no focal aggregation of glial cells at the implantation site for all time ranges. Astrocytosis (an increase in the number of astrocyte cells) and microglial activity at the cortical surface are within normal limits, indicating no overt immune reaction to the device and its by-products. Although the transdermal wiring does not noticeably affect animal behavior (see FIG. 36), a miniaturized, fully implantable wireless communication system might offer advantages, by removing the possibility of secondary infection at the wires. A wireless system constructed mostly, but not entirely, of resorbable materials (˜85% by mass and ˜86% by volume)—using an advanced near-field communication-technology approach, with fully bioresorbable metal coils, substrates and encapsulation layers—is provided in FIG. 5A-5H and FIGS. 37A-42.

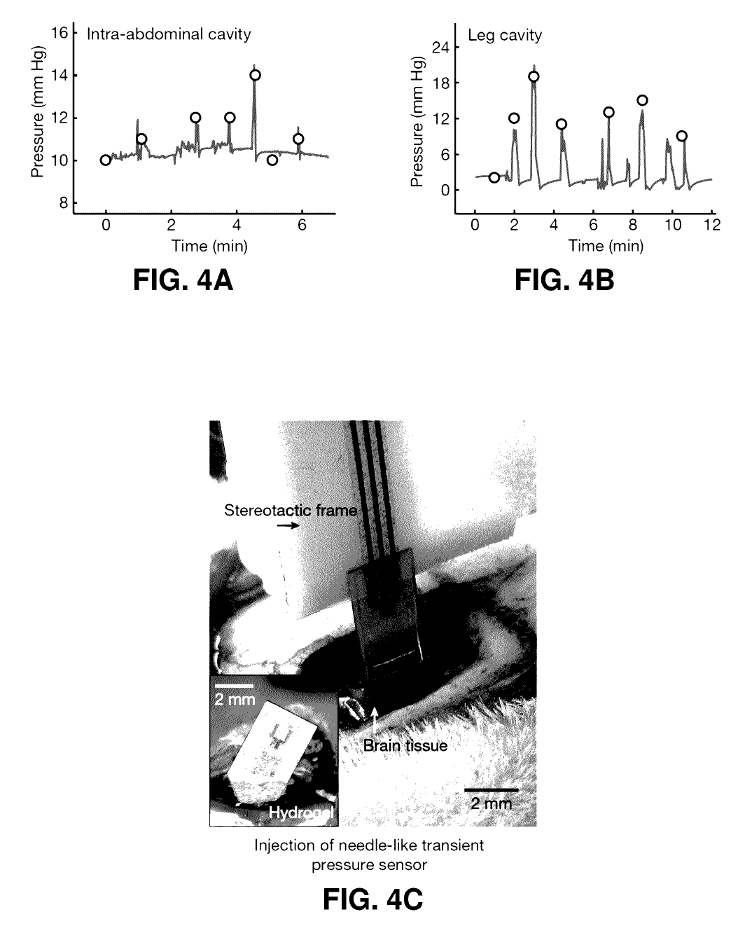

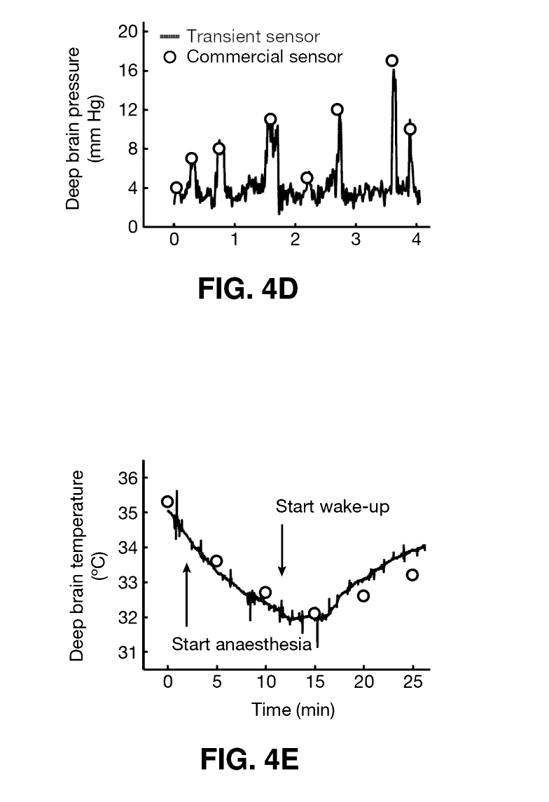

Given that these devices function successfully in the intracranial space, they could also be used in other organs and body compartments. As an example, FIGS. 4A and 4B illustrate ICP monitoring using the same bioresorbable device in modes with relevance to acute abdominal compartment syndrome and acute compartment syndrome of the extremity. Furthermore, modifying the devices to allow them to be injected deep into tissues can address other needs in clinical treatment. For example, monitoring physiological parameters of the deep brain with intraparenchymal sensors could yield data that are unavailable from the surface or the intracranial space. In addition, because electrophysiological and metabolic abnormalities often emanate from infarcts, contusions and haematomas that damage adjacent intact tissue, sensors of pressure, temperature, pH and other physical/chemical parameters that are placed into the parenchyma within the blood-deprived (ischaemic) penumbra could advance our knowledge of secondary brain injury. Such considerations apply not only to injured brain tissue, but also to acute or chronic ischaemia that threatens the heart, limbs, intra-abdominal organs or grafts.

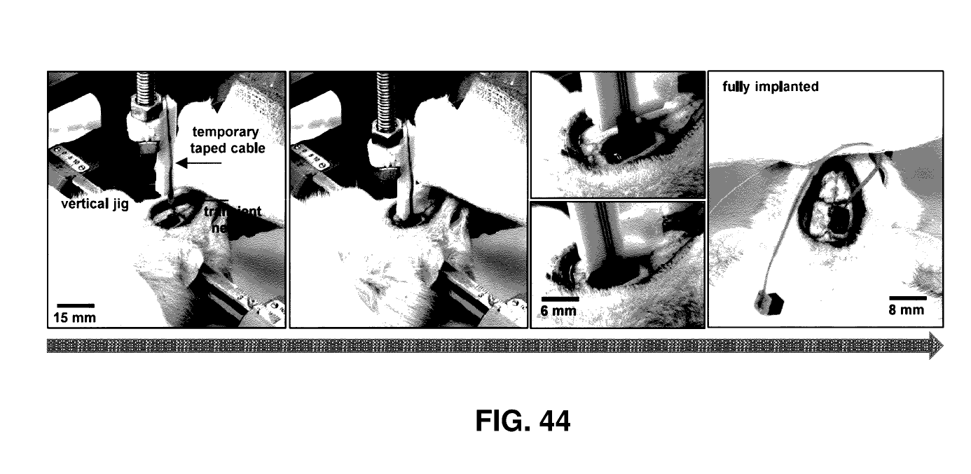

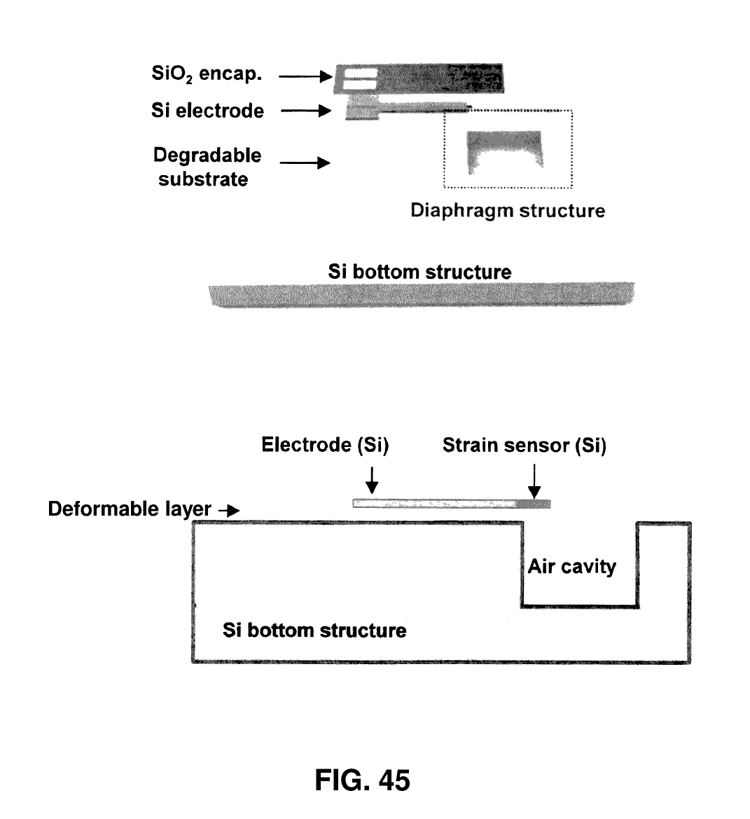

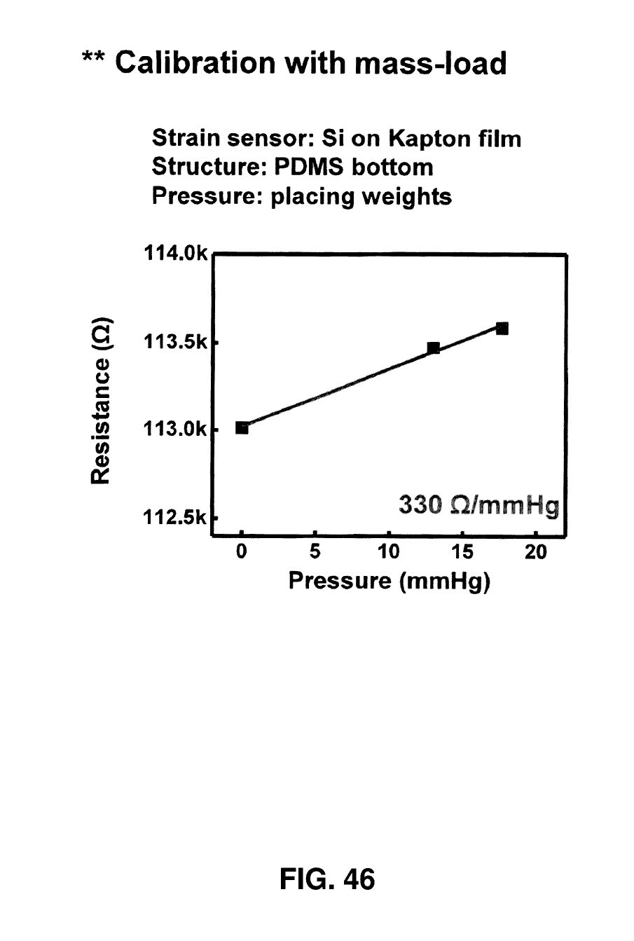

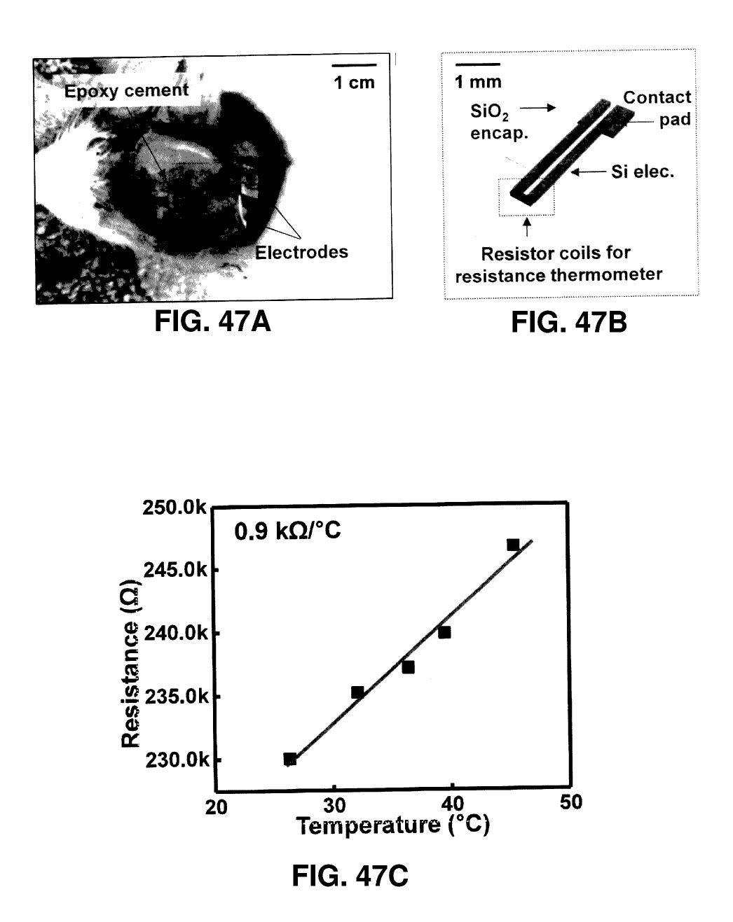

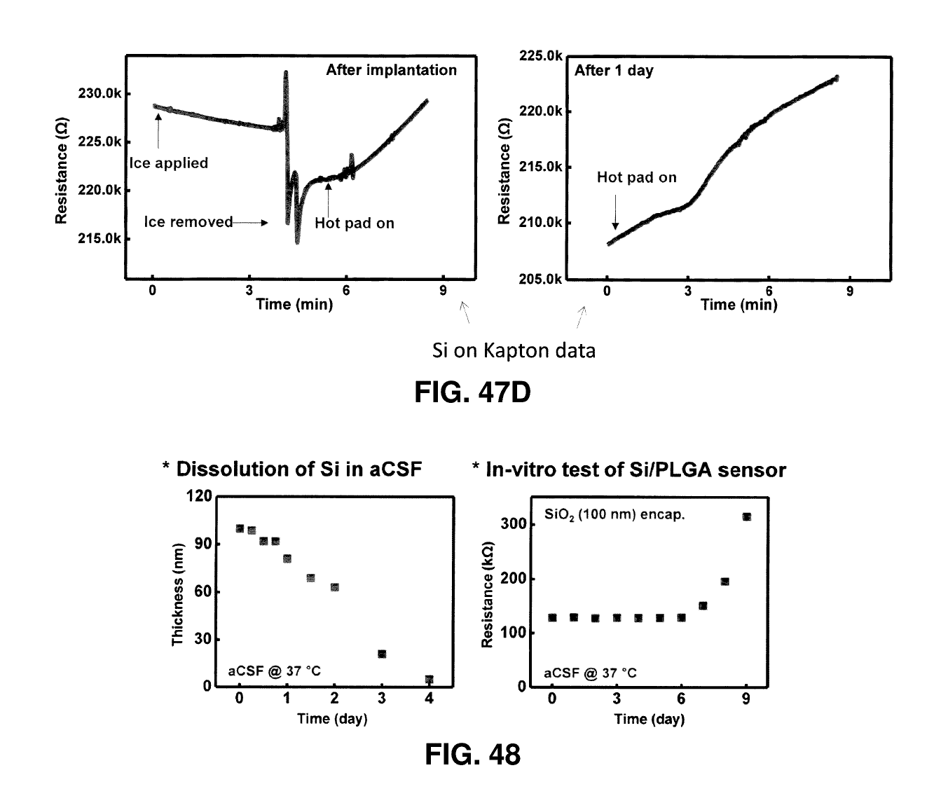

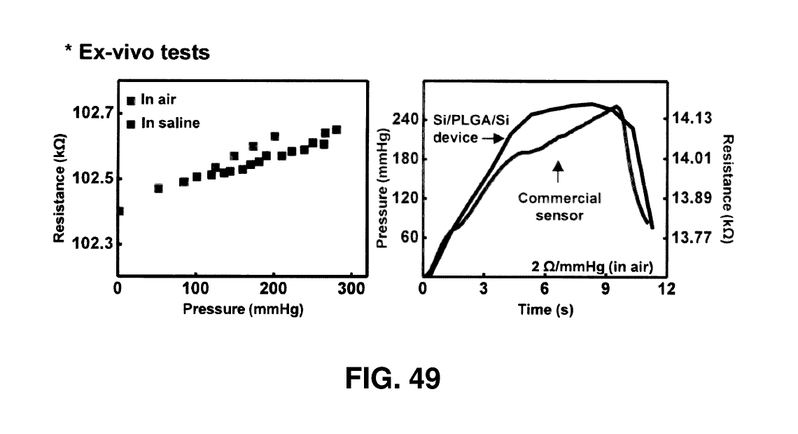

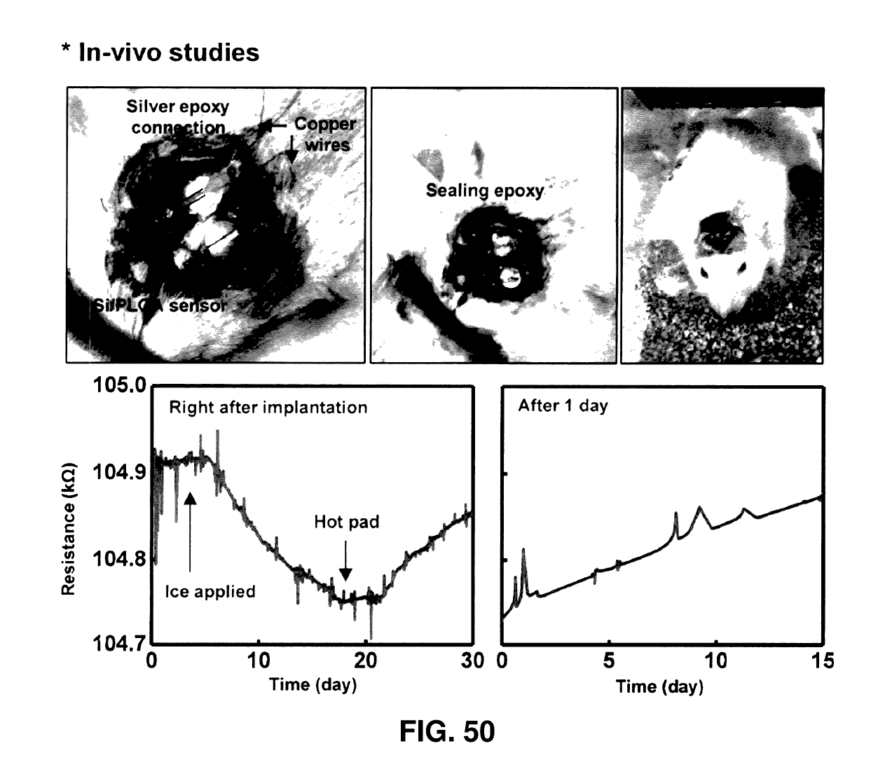

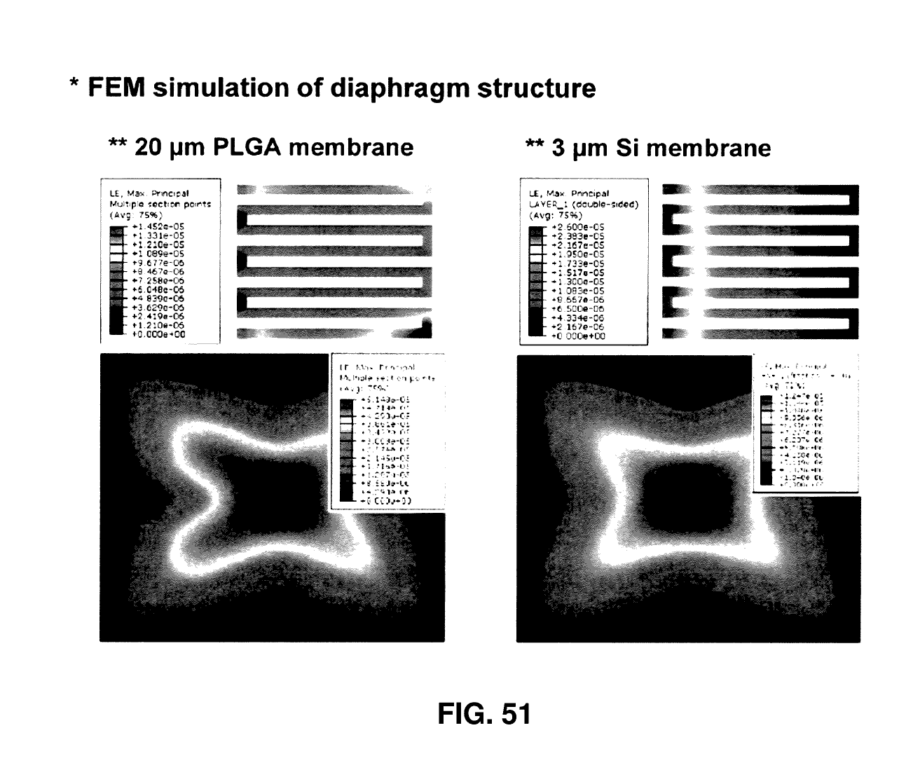

Modifying the geometry of the supporting structures introduced in FIG. 1A-1C enables delivery of bioresorbable sensors into the depths of brain tissue, for direct measurements of injury or status. FIG. 4C shows an example that integrates a bioresorbable ICP sensor onto a magnesium foil, formed with a tip region that allows injection into tissues of interest (FIG. 43A-43B). Mounting the device on a stereotactic frame and fixture allows accurate positioning and controlled penetration (FIG. 44). FIGS. 4D and 4E summarize pressure and temperature data collected at a site about 5 mm beneath the surface of the rat brain. The Valsalva maneuver yields data that quantitatively agree with those obtained using conventional sensors at a similar location. The device detected changes in temperature during anaesthesia (the temperature decreased, owing to reduced blood circulation) and waking up (the temperature returned to normal), as expected of intraparenchymal tissue.

The biomedical sensors reported here facilitate wireless data collection in body cavities and in deep tissues, with platforms that are fully bioresorbable, thereby allowing patients to be monitored until homeostasis has been achieved, and avoiding the risks associated with chronically implanted devices or their removal. In vivo and in vitro experiments demonstrate precision measurements of pressure, temperature, motion, flow, thermal properties and pH, with possible extensions to biomolecular binding events. These features will be useful in diagnosing and treating a diverse range of medical conditions, from acute traumatic injuries such as extremity compartment syndrome, to chronic medical diseases such as diabetes. The materials, manufacturing methods and design layouts are relevant to many other sensor modalities, with the potential for co-integration of advanced silicon-based integrated circuits, radio communication technologies, power supply and energy harvesters—each adapted from advances in transient electronics. Thus, these devices are useful for sensing, recording, stimulating, and electrical control for medical monitoring and treatment, not only for the body regions explored here but also for areas such as the cardiac space and spinal system. Applying these technologies into clinical settings provides patients and medical professionals with a vital set of tools for combating human disease.

Methods:

Fabrication of Bioresorbable Silicon Pressure Sensors:

Fabrication involved integration of silicon-based, piezoresistive sensing elements onto substrates of PLGA, bonded over cavities etched into the surfaces of nanoporous Si (np-Si) substrates or magnesium foils. Solid-state diffusion of boron yielded highly doped p-type monocrystalline silicon nanomembranes (Si-NMs) on silicon-on-insulator (SOI) wafers (top silicon ˜300 nm thick, p-type; SOITEC, France). Eliminating the buried oxide with hydrofluoric acid allowed transfer of the Si-NMs onto a bilayer of D-PI (diluted polyimide (poly(pyromellitic dianhydride-co-4,4′-oxydianiline)), ˜200 nm)/PMMA (poly(methyl methacrylate), ˜300 nm) on temporary silicon carrier substrates. Photolithography and etching patterned the Si-NMs into structures with serpentine designs. Electron-beam evaporation and spin-casting defined uniform layers of SiO₂ (˜100 nm) and D-PI, respectively, to serve the purpose of passivation. Selective dry etching through all of the layers (D-PI/SiO₂/D-PI/PMMA) formed a mesh structure that enabled release in acetone, for transfer to a film of PLGA (˜30 μm). Heating these films to temperatures near the glass transition of the PLGA (65° C.) and laminating them onto np-Si substrates (or magnesium foils, ˜60-80 μm) with square regions of etched relief (˜30-40 μm) formed sealed air cavities upon cooling to room temperature. Additional details appear hereinbelow.

Calibration of the Pressure Response:

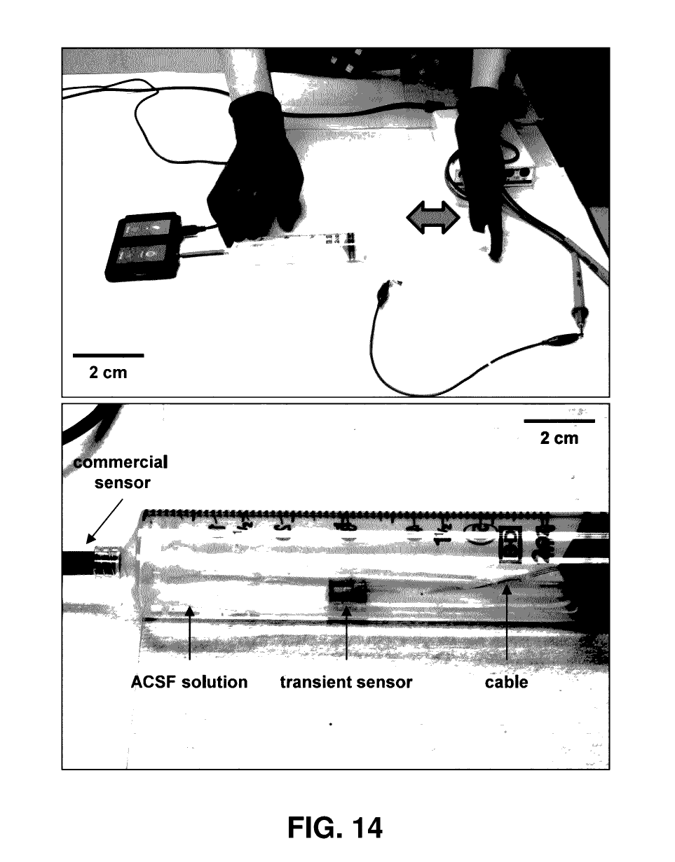

Responses of commercial sensors under environments similar to those in the intracranial cavity allowed absolute pressure calibration for the bioresorbable devices. The experiments involved placing a bioresorbable pressure sensor inside the barrel of a syringe partially filled with ACSF (Ecocyte BioScience, USA) and with a commercial sensor (NeuLog, USA) located at its open end (orifice). Moving the plunger component of the syringe allowed reversible access to well controlled pressures throughout a range relevant to intracranial monitoring (FIG. 14). Comparison of the electrical resistance of the bioresorbable sensor (via data acquisition (DAQ) system USB-4065, National Instruments, USA) with pressures from the commercial sensor yielded calibration curves. Additional data appear in the Supplementary Information.

Connections to Wireless Data-Transmission Systems:

Laser cutting of foils of molybdenum (˜10 μm thick) or magnesium (˜50 μm thick) yielded dissolvable narrow metal strips (that is, interconnection wires, 80 μm×30 mm). Pressing these wires against PLGA substrates using a PDMS stamp at 65° C. embedded them into the surface of the PLGA. Sputter deposition of molybdenum (˜2 μm) through high-resolution stencil masks (12.5 μm, Kapton; Dupont, USA) yielded electrical connections between the wires and contact pads on the PLGA (FIG. 25). The opposite ends of the wires connected to externally mounted wireless communication systems (Pinnacle Technology, USA) (FIG. 26).

Evaluation of the Kinetics of Device Dissolution:

Measurements of time-dependent changes in the thicknesses of square (100 μm×100 μm) Si-NMs (˜200 nm thick), electron-beam evaporated layers of SiO₂ (˜100 nm), free-standing nanoporous silicon substrates (np-Si, ˜80 μm) and magnesium foils (˜80 μm) due to immersion in ACSF at body temperature (37° C.) established the dissolution kinetics of the key materials. Removing samples from the ACSF every other day, rinsing them with deionized water, and measuring the thicknesses by profilometry (Dektak, USA) yielded the dissolution rate, as in FIG. 21. Sealed reservoirs of PDMS with viewing windows allowed for observation of dissolution behaviour at the level of the completed devices. These engineered structures included access channels around the periphery to allow passive fluid exchange and diffusion with a surrounding bath (FIG. 22).

In Vivo Evaluation:

Studies are performed in strict accordance with the recommendations in the Guide for the Care and Use of Laboratory Animals of the National Institutes of Health. The protocol was approved by the Institutional Animal Care and Use Committee (IACUC) of Washington University in St Louis (protocol number 20140207). Male Lewis rats weighing 250-350 g (Charles River, Wilmington, Mass.) received subcutaneous injections of buprenorphine hydrochloride (0.05 mg kg⁻¹; Reckitt Benckiser Healthcare Ltd, USA) for pain management, and of ampicillin (50 mg kg⁻¹; Sage Pharmaceuticals, USA) to prevent infection at the implantation site before the surgical process. Animals were anaesthetized with isoflurane gas and held in a stereotaxic frame for the duration of the surgical procedure and measurements. Opening a craniectomy and dural, implanting bioresorbable sensors on the cortical surface, sealing the craniectomy with a PLGA sheet (˜80 μm thick) and/or biodegradable surgical glue, and suturing the skin implanted the fully resorbable biosensing system in intracranial space. Comparison testing with a clinical intracranial pressure sensor (Integra LifeSciences, USA) and commercial thermistor (DigiKey Electronics, USA) implanted in parallel to bioresorbable sensors demonstrated the functionality of the bioresorbable sensors. To implant the injectable device, the same procedure of opening a craniectomy and dural was performed. Injecting needle-shaped biosensors into the brain parenchyma (˜5 mm deep) with a stereotactic frame and arm enabled monitoring of pressure and temperature in the deep-brain parenchyma. Additional details on the manual operation of pressure/temperature changes, the immunohistochemistry tests, and the surgical process and measurement at the intra-abdominal cavity and lower extremities appear hereinbelow. The immunohistochemistry tests used five individual rats per stage (2, 4 and 8 weeks) and device type (np-Si and magnesium-foil substrates). In vivo functionality tests of pressure and temperature sensors involved three trials using different batches of devices and animals, to establish reproducibility.

Implantable Near-Field-Communication Wireless System:

The sensor introduced in FIG. 2A can be integrated with sub-dermal wireless data-transmission systems, constructed largely of bioresorbable materials, via thin, bioresorbable wires that pass through the skull. FIGS. 5A and 5B show an illustration of a chip-scale, near-field-communication (NFC) technology that includes bioresorbable coils, polymer substrates, encapsulation layers and resistors, a partially bioresorbable NFC chip, and non-resorbable capacitors, and a picture of this system integrated with a bioresorbable pressure sensor via biodegradable wiring. Here, micro-patterned magnesium coils (50 μm thick, outer diameter 15 mm) allow inductive coupling to an external data reader for power transfer and data transmission. A silicon-based logic chip (RF430FRL152H, Texas Instruments, USA; 4 mm×4 mm×˜300 μm) captures the measured data at a high acquisition rate, then digitizes and processes the information for transmission to the external reader. Passive components include Si-NM resistors and capacitors. PLGA serves as the substrate and electrical passivation layer. FIG. 5C summarizes the operating principles. The external reader wirelessly delivers power for operating the logic chip and provides the small currents needed to assess the response of the piezoresistive and thermoresistive sensors. In particular, changes in resistance associated with changes in pressure and temperature register as voltages that can be recorded and transmitted to the external reader by NFC chip through the associated coil antenna. This NFC system is far more sophisticated than a conventional radio-frequency-identification (RFID) tag. Here, a single chip platform provides all of the computing functionality needed for high-speed data recording, real-time software filtering, and wireless transmission of sensor outputs as captured with an on-board 14-bit analogue-to-digital converter.

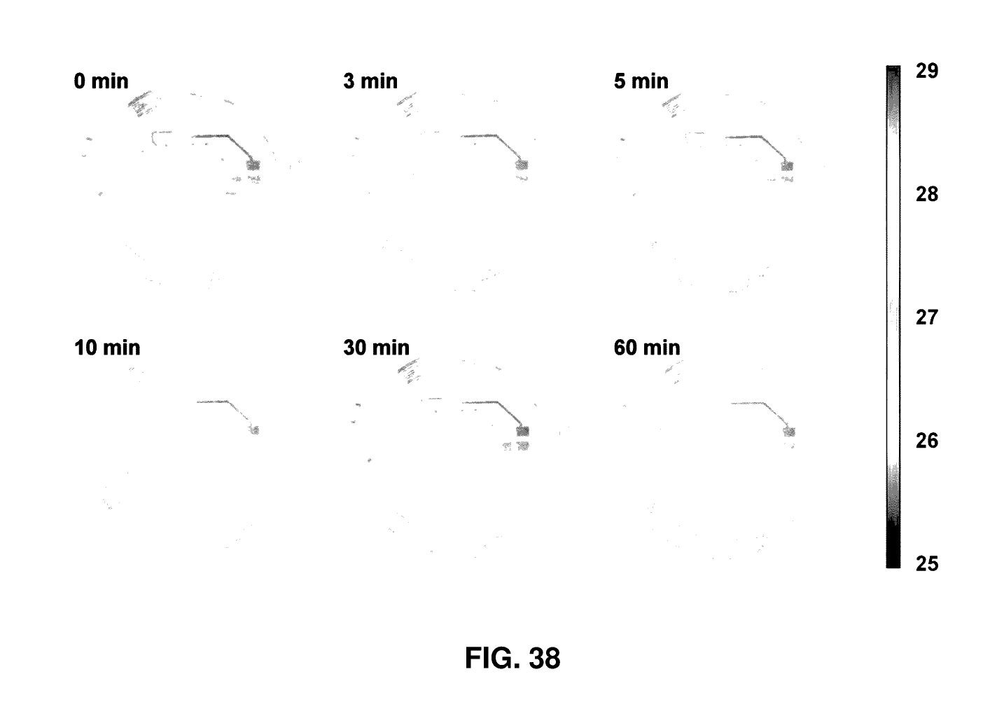

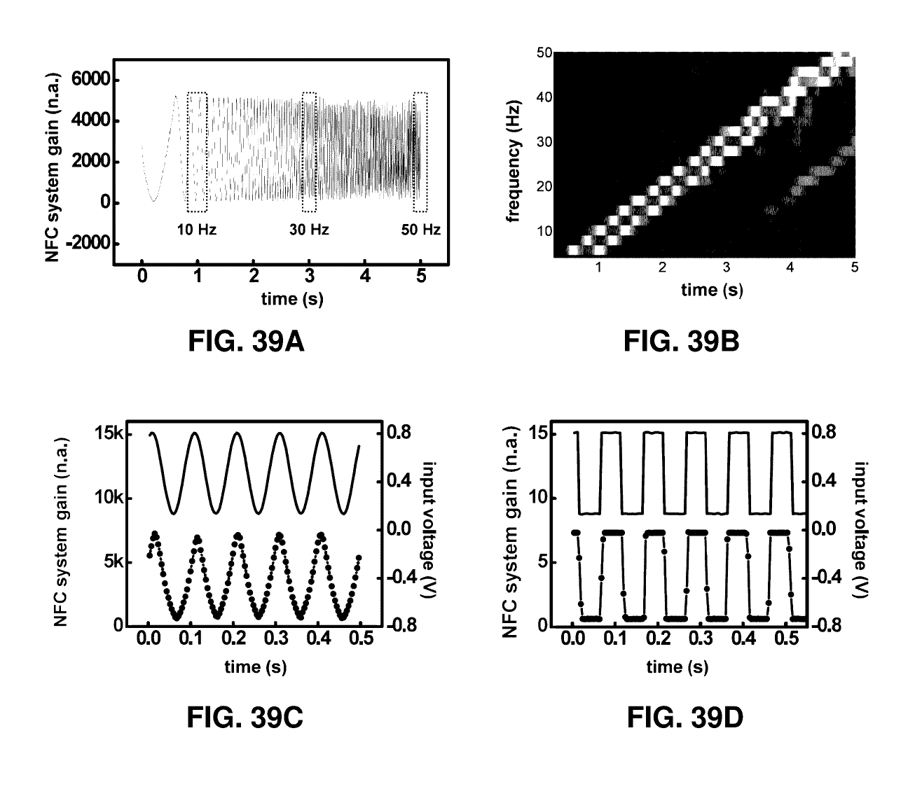

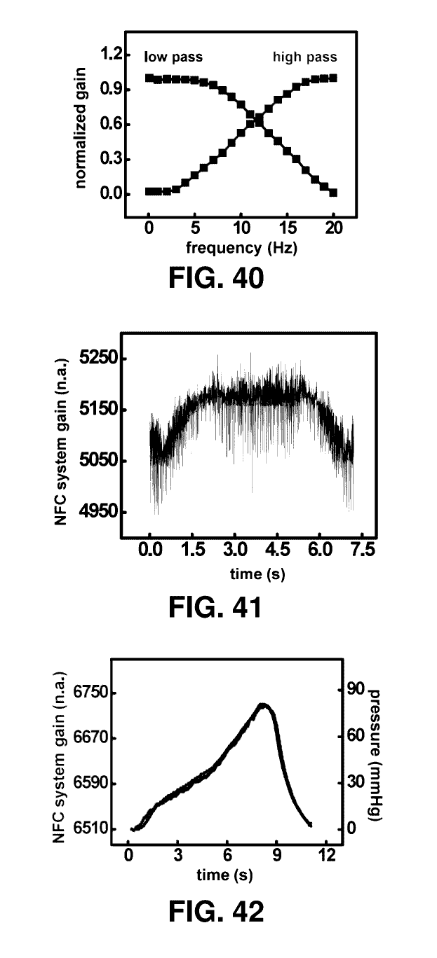

This system communicates through biofluids and tissue with little loss, owing to the use of magnetic coupling in a relatively low-frequency band (13.56 MHz; FIG. 37), consistent with negligible heating associated with system operation (FIG. 38). These characteristics enable communication distance of up to 25 mm through biological tissue. The high-speed, programmable operation of the NFC chip is critical to overall operation. FIG. 39A presents examples of data-acquisition rates of up to 250 Hz, via recordings of oscillating voltages (sine wave) with frequencies from 1 Hz to 50 Hz. Spectrograms and other related data appear in FIGS. 39B-39D. These high sampling rates allow efficient operation of digital filtering algorithms, and they also foreshadow the ability to measure biosignals such as EEG, ECoG and ECG. FIG. 40 demonstrates the response of real-time high/low-pass filter function achieved by software programming for on-board computation with the NFC chip. FIG. 41 shows two-channel operation of the system with/without this type of filtering during sensing. This integrated system provides wireless operation that compares quantitatively with that of a commercial wired sensor (FIG. 42).

The wireless module is largely bioresorbable, as illustrated in FIG. 5D through images at various stages of dissolution in ACSF at 60° C. The magnesium coils, electrodes, interconnects and silicon resistors (240 mg; ˜85% of the total mass of the NFC system) dissolve fully after 14 days. Here, the NFC chip is not bioresorbable; but fully bioresorbable complementary metal-oxide semiconductor (CMOS) circuit technologies offer the potential for constructing bioresorbable chips. In particular, recently reported schemes demonstrate that modest modifications to otherwise conventional semiconductor-manufacturing techniques allow the use of foundry fabrication facilities for construction of bioresorbable CMOS. Even with the examples presented here, where the NFC chip is not fully bioresorbable, the associated implantation strategy minimizes risk by locating the hardware subdermally on the skull, outside the intracranial space, thereby allowing rapid, facile extraction.

In this overall architecture, the fully bioresorbable sensors reside in the intracranial space, while the NFC system resides extracranially within the subgaleal layer of the scalp. Fine, dissolvable wiring provides electrical interconnections through a burr hole in the skull, sealed with a bioresorbable surgical glue. After completing the subgaleal closure, the wireless system and sensor are fully implanted. FIGS. 5E-5H show a diagram of the implantation strategy, surgical process, and wireless in vivo intracranial pressure and temperature results measured in a rat model. Here, all of the components in the intracranial region are fully bioresorbable. The non-bioresorbable components of the system remain extra-axial within the scalp, thereby minimizing the risk of provoking pathological neuroinflammation in the intracranial space. In addition, the relative material safety (as judged by the US Food and Drug Administration (FDA) class) of, for example, a subdermally implanted encapsulated non-resorbable device (such as an RFID chip) is similar to that of a titanium fixation screw. Removal of an extra-axial component involves a much lower risk than intracranial surgery. Intracranial pressure and temperature values measured in the rat model using the NFC system are comparable to those captured using commercial wired sensors.

Fabrication of Fully Implantable NFC Wireless System:

The magnesium foil was patterned on the PDMS by using photolithography and etching with dilute hydrochloric acid (deionized water:HCl=15:1). Transfer printing of the patterned magnesium foil onto a film of PLGA (˜150 μm) formed the inductive coil and electrode. The Si-NM resistor was formed on the SOI wafer by doping with phosphorus at 950° C. and patterning the top silicon (˜300 nm thick) into the trace. Undercutting the buried oxide with hydrofluoric acid and transfer printing Si-NM on PLGA formed the resistor of NFC system. Laminating the top PLGA (˜150 μm) and heating it at 65° C. yielded the passivation layer. Biodegradable conductive W paste served to interconnect the NFC wireless system to the metal wire (molybdenum or magnesium).

### REFERENCES

1. Poole, J. E. Present guidelines for device implantation: clinical considerations and clinical challenges from pacing, implantable cardiac defibrillator, and cardiac resynchronization therapy. Circulation 129, 383-394 (2014).
2. Brain, T. F. Guidelines for the management of severe traumatic brain injury. VI. Indications for intracranial pressure monitoring. J. Neurotrauma 24, S37-S44 (2007).
3. Chamis, A. L. et al. Staphylococcus aureus bacteremia in patients with permanent pacemakers or implantable cardioverter-defibrillators. Circulation 104, 1029-1033 (2001).
4. Hall-Stoodley, L., Costerton, J. W. & Stoodley, P. Bacterial biofilms: from the natural environment to infectious diseases. Nature Rev. Microbiol. 2, 95-108 (2004).
5. Maytin, M. & Epstein, L. M. Lead extraction is preferred for lead revisions and system upgrades. Circ. Arrhythm. Electrophysiol. 3, 413-424 (2010).
6. Boutry, C. M. et al. Towards biodegradable wireless implants. Phil. Trans. R. Soc. A 370, 2418-2432 (2012).
7. Ott, K. et al. Retained intracranial metallic foreign bodies: report of two cases. J. Neurosurg. 44, 80-83 (1976).
8. Vajramani, G. V. et al. Persistent and intractable ventriculitis due to retained ventricular catheters. Br. J. Neurosurg. 19, 496-501 (2005).
9. Hwang, S.-W. et al. A physically transient form of silicon electronics. Science 337, 1640-1644 (2012).
10. Irimia-Vladu, M. “Green” electronics: biodegradable and biocompatible materials and devices for sustainable future. Chem. Soc. Rev. 43, 588-610 (2014).
11. Bettinger, C. J. & Bao, Z. Organic thin film transistors fabricated on resorbable biomaterial substrates. Adv. Mater. 22, 651-655 (2010).
12. Luo, M., Martinez, A. W., Song, C., Herrault, F. & Allen, M. G. A microfabricated wireless RF pressure sensor made completely of biodegradable materials. J. Microelectromech. Syst. 23, 4-13 (2014).
13. Brogan, M. E. & Manno, E. M. Treatment of malignant brain edema and increased intracranial pressure after stroke. Curr. Treat. Options Neurol. 17, 327 (2015).
14. Liu, C. Foundations of MEMS Ch. 6 (Prentice Hall, London, 2011).
15. Moseley, P. T. & Crocker, J. Sensor Materials Ch. 4 (Institute of Physics Publishing, London, 1994).
16. Chang, H. et al. DNA-mediated fluctuations in ionic current through silicon oxide nanopore channels. Nano Lett. 4, 1551-1556 (2004).
17. Zheng, G., Patolsky, F., Cui, Y., Wang, W. U. & Lieber, C. M. Multiplexed electrical detection of cancer markers with nanowire sensor arrays. Nature Biotechnol. 23, 1294-1301 (2005).
18. Stern, E. et al. Label-free immunodetection with CMOS-compatible semiconducting nanowires. Nature 445, 519-522 (2007).
19. Gentile, P., Chiono, V., Carmagnola, I. & Hatton, P. V. An overview of poly(lactic-co-glycolic) acid (PLGA)-based biomaterials for bone tissue engineering. Int. J. Mol. Sci. 15, 3640-3659 (2014).
20. Hwang, S.-W. et al. Dissolution chemistry and biocompatibility of single-crystalline silicon nanomembranes and associated materials for transient electronics. ACS Nano 8, 5843-5851 (2014).
21. Kang, S.-K. et al. Dissolution behaviors and applications of silicon oxides and nitrides in transient electronics. Adv. Funct. Mater. 24, 4427-4434 (2014).
22. Yin, L. et al. Dissolvable metals for transient electronics. Adv. Funct. Mater. 24, 645-658 (2014).
[23] Uslaner, J. M. et al. T-type calcium channel antagonism produces antipsychotic-like effects and reduces stimulant-induced glutamate release in the nucleus accumbens of rats. Neuropharmacol. 62, 1413-1421 (2012).
24. Barth, K. N. M., Onesti, S. T., Krauss, W. E. & Solomon, R. A. A simple and reliable technique to monitor intracranial pressure in the rat: technical note. Neurosurgery 30, 138-140 (1992).
25. Haure, P., Cold, G. E., Hansen, T. M. & Larsen, J. R. The ICP-lowering effect of 10° reverse Trendelenburg position during craniotomy is stable during a 10-minute period. J. Neurosurg. Anesthesiol. 15, 297-301 (2003).
26. Morgan, P. W. Structure and moisture permeability of film-forming polymers. Ind. Eng. Chem. 45, 2296-2306 (1953).
27. Meldrum, D. R. et al. Prospective characterization and selective management of the abdominal compartment syndrome. Am. J. Surg. 174, 667-673 (1997).
28. Olson, S. A. & Glasgow, R. R. Acute compartment syndrome in lower extremity musculoskeletal trauma. J. Am. Acad. Orthop. Surg. 13, 436-444 (2005).
29. Stiefel, M. F. et al. Reduced mortality rate in patients with severe traumatic brain injury treated with brain tissue oxygen monitoring. J. Neurosurg. 103, 805-811 (2005).
30. Timofeev, I. et al. Extracellular brain pH with or without hypoxia is a marker of profound metabolic derangement and increased mortality after traumatic brain injury. J. Cereb. Blood Flow Metab. 33, 422-427 (2013).
31. Suehiro, E. et al. Diverse effects of hypothermia therapy in patients with severe traumatic brain injury based on the computed tomography classification of the traumatic coma data bank. J. Neurotrauma 32, 353-358 (2015).
32. Mittal, R. et al. Use of bio-resorbable implants for stabilization of distal radius fractures: the United Kingdom patients' perspective. Injury 36, 333-338 (2005).
33. Ye, T. et al. Management of grade Ill open dislocated ankle fractures: combined internal fixation with bioabsorbable screws/rods and external fixation. J. Am. Podiatr. Med. Assoc. 101, 307-315 (2011).
34. Yin, L., Bozler, C., Harburg, D. V., Omenetto, F. & Rogers, J. A. Materials and fabrication sequences for water soluble silicon integrated circuits at the 90 nm node. Appl. Phys. Lett. 106, 014105 (2015).
35. Huang, X. et al. Biodegradable materials for multilayer transient printed circuit boards. Adv. Mater. 26, 7371-7377 (2014).

1. Fabrication of Nanoporous Si (or Mg Foil) Trench

Nanoporous Si (np-Si) and Mg foil served as mechanical supports for microelectromechanical systems (MEMS). Free-standing np-Si (˜80 μm thick) was prepared from double-side polished, highly doped p-type Si wafers (0.001-0.005 Ω·cm, University Wafers, USA) at a current density 160 mA·cm⁻², as previously reported. Lamination delivers the np-Si onto a layer of PDMS spin cast on a glass slide, for deposition of SiO₂ (˜300 nm) by PECVD. Patterning and etching of the SiO₂ and np-Si yielded a trench in the np-Si. As an alternative to np-Si, a commercial Mg foil (˜100 μm thick, Goodfellow, USA) can be thinned, patterned, and wet etched by a mixture of acetic acid (CH₃COOH, Transene Company Inc., USA) and deionized (DI) water (20 ml: 250 ml), to create a trench (˜40 μm depth) in the processed Mg foil (˜80 μm thick). The PLGA is transfer printed on the np-Si (or Mg foil) with a micro-tip patterned PDMS stamp. The manufactured devices on PLGA substrates were then integrated with np-Si (or Mg foil) near the glass transition temperature (T_{g}, ˜65° C.) for 5 min on a hot plate.

2. Calibration of Biodegradable Biosensors

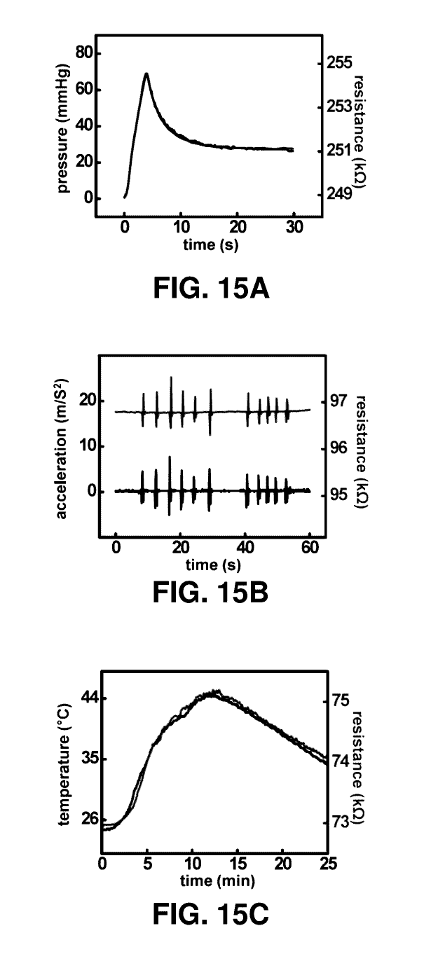

Accelerometer—Calibration involved measurements of a biodegradable accelerometer and a commercial device (NeuLog, USA) moved rapidly up and down in the vertical direction. The calibration approach to connect measured changes in resistance to acceleration was similar to that used for the pressure sensor. Temperature sensor—Real-time measurements of the change in resistance of a biodegradable sensor and of temperature using a commercial sensor (NeuLog, USA) submerged in ACSF yielded the calibration curves (FIG. 15). Flow meter—The device was placed in a water bath with constant flow rate. While 1 mA (DC current source; Model 6220, Keithley, USA) was applied to the thermal actuator, the resistances of the two temperature sensors were measured using a data acquisition (DAQ) system (USB-4065, National Instruments, USA). Thermal conductivity/diffusivity sensor—The thermal conductivity of an aqueous solution was measured as follows. A current (500 μA) was supplied to the resistive element by a programmable DC current source (Model 6220, Keithley, USA) for 1 s. The time-dependent voltage across the element was then sampled at 100 V/s using a 22-bit programmable digital multimeter (USB-4065, National Instruments, USA). The voltage and current values allowed calculation of the time-dependent resistance of the device, which, in turn, is proportional to temperature. pH sensor—Si nanoribbons (Si NRs) were exposed by ultraviolet induced ozone for 3 min and immersed in a 1% ethanol solution of 3-aminopropyltriethoxysilane (APTES, Sigma-Aldrich, USA) for 20 min. After thorough rinsing with ethanol three times, Si NRs were annealed at 60° C. for 10 min to functionalize their surfaces. An Ag/AgCl reference electrode was placed in the center of the solutions, and a floating gate voltage defined the quiescent conductance of the Si NRs. Changes in conductance were measured during partial immersion of the device in phosphate buffer solution (Sigma Aldrich, USA) with various pH between 2 and 10. After a short period of stabilization, a semiconductor analyzer (4155C, Agilent, USA) recorded the conductance of the Si NRs for ˜50 s in the solutions.

3. Operating Principles of Mechanical/Physical/Chemical Sensors

Pressure sensor—The deformable diaphragm structure in FIG. 1A provides a highly sensitive pressure response. The average strain in the Si-NM serpentine structure on PLGA induced by applied pressure causes a piezoresistive electrical response. Under intracranial pressure, both the np-Si mechanical support and PLGA diaphragm would deform. Three-dimensional finite element analysis (3D-FEA), however, suggests that the deformation of the np-Si mechanical support is negligible in comparison to that of PLGA diaphragm, indicating a reasonable simplification to clamp all four edges of PLGA diaphragm in the current study. In the numerical analysis, an 8-node hexahedral solid element C3D8R and a quadrilateral shell element S4R were used for the diaphragm and Si-NM piezoresistive sensor, respectively. The ideal elastic constitutive relation describes the mechanical behavior of the Si and PLGA. To maximize the sensor sensitivity (i.e., average strain in the Si-NM piezoresistive sensor) to applied pressure, the center of the edge of the diaphragm was chosen as the location for the sensor. The average strain at different positions of diaphragm shown in FIG. 10 rationalizes this choice. The sensitivity also depends on the size of PLGA diaphragm. For a given area, 3D-FEA results indicate that the optimum is achieved when the PLGA diaphragm is close to a square in shape (see FIG. 11). FIG. 12 provides a comparison of experimental and FEA results for change in resistance with pressure. The following equation provides the relationship between the change in resistance due to piezoresistivity through a gauge factor (G):

R=R₀(1+G∈), (1)

where ∈ is the average strain on the piezoresistive sensor, and R and R₀ are the change in resistance and resistance at zero pressure, respectively. Here, the average strain was calculated from 3D-FEA for pressure ranging from 0 to 70 mmHg. With R₀ as 249 kΩ, the gauge factor was estimated to be ˜30. Accelerometer—The pressure sensor platform can serve as an accelerometer where the sensor consists of a cantilever with a rigid proof mass m attached at its distal end (FIG. 16). Under a given acceleration a, the force experienced by the proof mass generates a bending moment at the fixed end of the beam, M=mad, where d is the distance from the center of the proof mass to the fixed end. This results in a strain at the location where Si-NM piezoresistive coil resides ∈=Mt/(2EI), where EI=Ewt³/12 is the bending stiffness of the cantilever, and wand t are the width and thickness of the cantilever, respectively. Taken together with Equation (1), the acceleration sensitivity is tunable by changes of the elastic properties and the geometry via the following relationship a=ΔR/R(Ewt²)/(6Gmd). Temperature sensor—The resistance (or conductance) changes by an amount linearly proportional to temperature, according to:

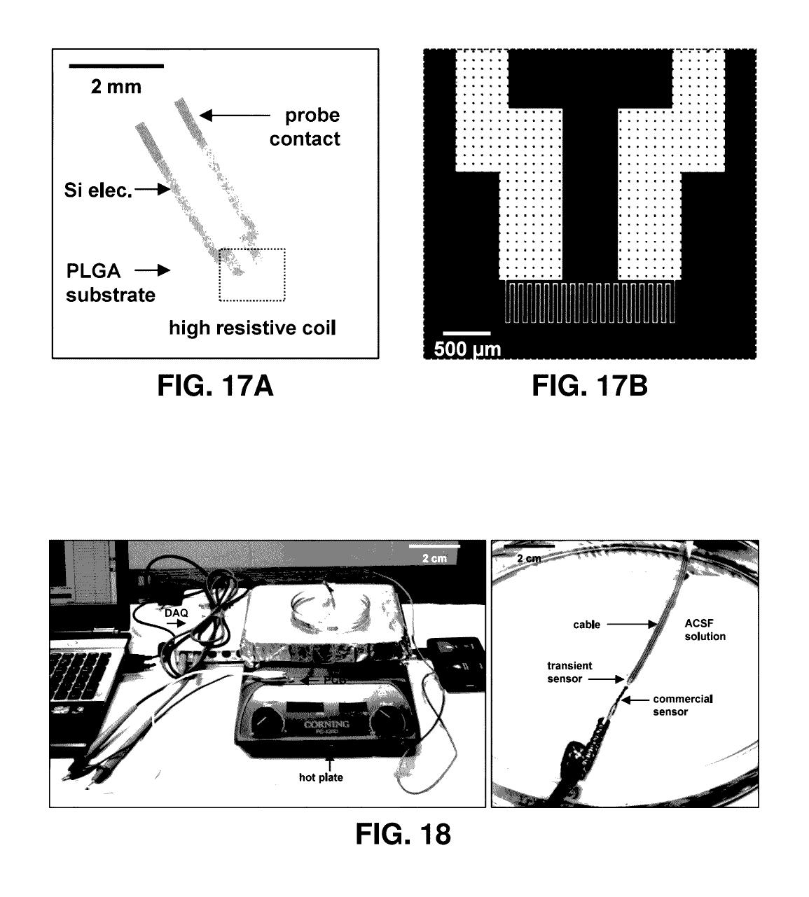

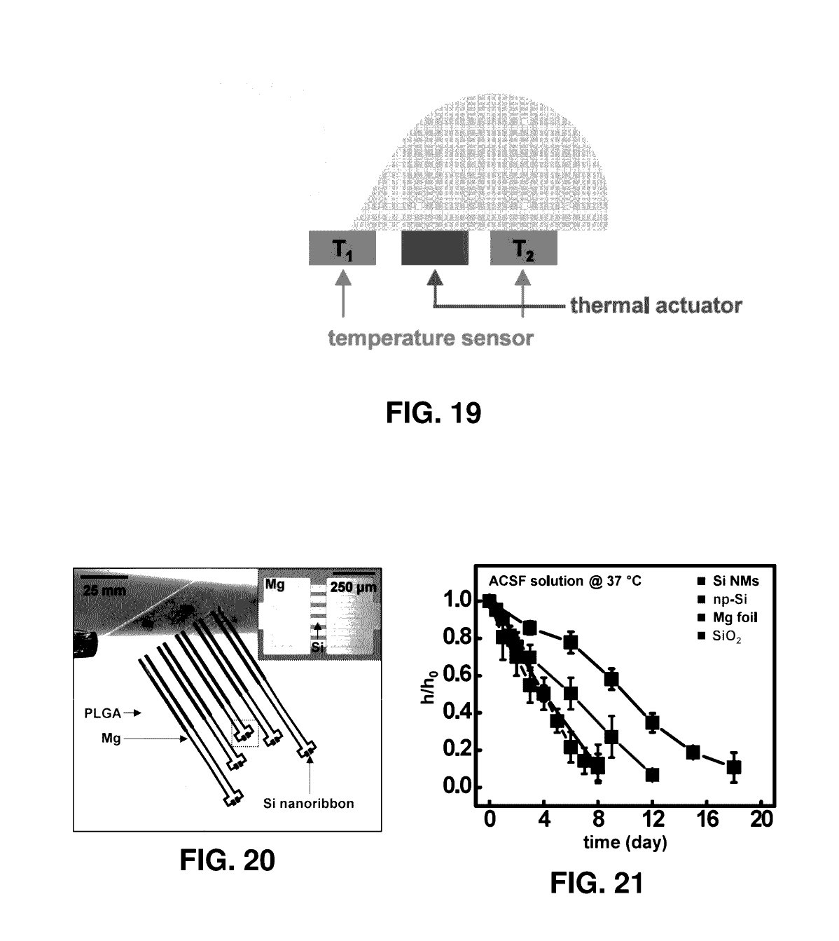

R=R₀(1+α(T−T₀)) (2)

where R and R₀ are the changed resistance and initial resistances, T and T₀ are the measured and initial temperatures, and α is the temperature coefficient of resistance (TCR). The TCR in FIG. 1H is 1×10⁻⁴, where doped Si has various TCR ranges depending on the type of dopants and doping concentration. A larger initial resistance increases the absolute change in resistance. As a result, highly dense serpentines, which dominate the total resistance, were adopted for the temperature sensors (see FIG. 17). Flow rate monitor—The flow rate monitor consists of one thermal actuator and two temperature sensors (see FIG. 19). The thermal actuator is placed in the middle of two temperature sensors, along the flow direction. When the actuator generates heat, a temperature difference appears between two temperature sensors, due to heat transfer mediated by the flow. The difference in temperature can be quantitatively correlated to flow rate. Thermal conductivity/diffusivity sensor—A sudden increase of the power applied to the resistive element induces a temperature increase in the resistor due to Joule heating. The time dynamics of the temperature increase is, in part, a function of the thermal transport properties of the surrounding fluid. As a result, analysis of the temperature transients immediately following the power increase allows determination of the thermal transport properties of the surrounding fluid or tissue. pH sensor—The functionalized surfaces of Si-NRs with both —NH₂ and —SiOH groups undergo protonation to —NH₃⁺ at low pH and deprotonation to —SiO⁻ at high pH. The resulting changes in the surface charge electrostatically gate the transport in the Si-NRs by depleting or accumulating charge carriers, resulting in a stepwise decrease in the conductance of the phosphorous-doped Si-NRs as the pH in aqueous solutions increases from 2 to 10 in distinct steps (see FIG. 20 for Si pH sensor).

4. Temperature Effects on Piezoresistivity of Pressure Sensors

Temperature affects the resistance and the coefficient of piezoresistivity. Therefore, calibration of these effects in piezoresistive-type pressure sensors enhances the accuracy of the pressure measurements. The temperature effect on resistance is described by Equation (2) in the previous section through the temperature coefficient of resistance (TCR, α). The temperature effect on piezoresistivity is described by the temperature coefficient of piezoresistivity (TCP, β)

π=π₀(1+β(T−T₀)), (3)

where π and π₀ are the final and initial piezoresistivity coefficients, and T and T₀ are the measured and initial temperatures, respectively. The temperature coefficient of piezoresistivity β of Si varies, depending on the dopants and doping concentration. Assuming the piezoresistivity is directly proportional to the gauge factor (G), the strain (∈) can be correlated with resistance (TCP) as

R_{(T,P)}=R_{(T,P=0)}(1+G∈)=R_{(T=0,P=0)}[1+α(T−T₀)]{1+G₀[1+β(T−T₀)]∈}, (4)

where R_{(T,P)} is the final resistance affected by temperature and pressure, R_{(T,P=0)} is the resistance when only temperature is applied, R_{(T=0,P=0)} is the initial resistance without changes in pressure nor temperature (usually 20 or 25° C.), G and G₀ are the final and initial gauge factors. With predetermined α, β, G₀ and R_{(T=0,P=0)}, the temperature effects on the resistance and piezoresitivity can be calibrated. FIG. 24 shows the resistance vs. pressure curve at the different temperatures measured by transient pressure sensors, giving α=0.5×10⁻³ and β=−2.2×10⁻³. It should be noted that a typical range of β of p-type Si is −2.7×10⁻³ to −1.6×10⁻³, for doping concentration between 5×10¹⁸ and 1×10²⁰/cm. Here the change in pressure sensitivity due to temperature change in this intracranial study (±5° C.) is about 1%, and hence negligible. The following simplified equation can then be used to calibrate the temperature effect on the base resistance:

R_{(T,P)}=R_{(T=0,P=0)}[1+α(T−T₀)](1+G₀∈). (5)

Optimizing the doping concentration to increase the sensitivity to pressure and minimize sensitivity to temperature, and/or using a Wheatstone-bridge type of design with four piezoresistive elements, are alternative routes to minimize the temperature effect.

5. Animal Behavior Test with Percutaneous Wires

The novel object recognition (NOR) task is used to evaluate cognition, particularly recognition memory, in rodent models of CNS disorders. We tested 6 controls and 6 rats with implanted percutaneous wires and their spontaneous tendency to spend more time exploring a novel object than a familiar one. The choice to explore the novel object reflects the use of learning and recognition memory. We observed no significant change in those with precautions implants as opposed to controls as shown in FIG. 36.

6. In Vivo Implantation of Bioresorbable Wireless Monitors for Intracranial Pressure and Temperature

Animals were anesthetized and held in a stereotaxic frame after analgesia and antibiotic prophylaxis. Incising along the dorsal midline of the head longitudinally and retracting the scalp allowed the visualization of bregma and lamdba. The craniectomy was fashioned utilizing a high speed drill on the right side of the rat's skull over somatosensory cortex. After placing the transient biosensors on the cortical surface, small pieces of saline soaked absorbable gelatin compressed sponge (Gelfoam®, Pfizer, USA) were applied. For the fully implantable NFC system, the craniectomy defect was sealed by degradable surgical glue (TISSEEL, Baxter Healthcare Co., USA), and the NFC system was placed on the outside surface of the skull. A subgaleal closure utilizing interrupted resorbable sutures sealed the surgical site with all device components fully implanted. For the transcutaneous bioresorbable wiring method, placing a PLGA sheet on the skull by covering the craniectomy defect and bonding the sides by degradable surgical glue formed the closed intracranial cavity while the wires were withdrawn outside. Finally, a subgaleal closure utilizing interrupted resorbable sutures while laying degradable wires out provides the fully resorbable interfaces with dissolvable metal wires. The plastic hat, which provides the protection from rat's movement and handling, was bonded on the sutured region with an epoxy. Placing a wireless transmitter (Pinnacle 8151 fixed-frequency 2-channel wireless potentiostat, Pinnacle Technology, USA) with connection of the degradable wires in the plastic hat allows the long range (>10 m) wireless monitoring of pressure and/or temperature. The battery-powered potentiostat delivered a 0.6 V potential to each channel at a 1 Hz sampling frequency, measured and amplified the delivered current with a gain of 100,000 digitized the reading, and transmitted the value over a 900 MHz wireless protocol to a computer-connected base station. Implanting a standard clinical ICP monitor, (Integra LifeSciences Co., USA) near the dissolvable sensor and sealing with a silicone polymer enabled the comparison of resorbable pressure sensor with a clinical ICP monitor. The commercial thermistor (DigiKey Electronics, USA) implanted at the site near the resorbable temperature sensor and plugged to a wireless transmitter provided the parallel monitoring of brain temperature with resorbable sensor. Post-operative care procedure based on the local protocol of the animal welfare regulations providing the rat's reasonable recovery and health movement in single-housed cage.

7. Characterization of the Near-Field Communication (NFC) System

The NFC chip operates using power delivered wirelessly by inductive coupling. The regulated working voltage ranges from 1.45 to 1.65 V. The chip includes a microcontroller (MSP430), with 2 kB, 4 kB and 8 kB of FRAM, SRAM and ROM, respectively. The system supports four channels of Sigma-Delta analog to digital converters, each with 14 bit resolution. The phase, impedance, and resonance frequency of the fully implantable NFC system was evaluated by near field coupling to a coil connected to an impedance analyzer (Agilent 4191A RF Impedance Analyzer, Agilent, USA). A signal generator (Keithley 3390, Keithley, USA) provides 10 Hz sine, square, ramp wave inputs to the NFC system. The input of the signal generator and the wireless measured signals from the NFC system were compared to assess the high speed acquisition capabilities. Finite impulse response filters were loaded into the chip and frequency responses were acquired by using the signal generator to produce sine wave inputs with frequencies between 0.1 to 20 Hz.

8. Synthesis/Hydrolysis Chemistry, Dissolution Kinetics, Water Permeability, Biocompatibility of Polyanhydride Encapsulation

A biodegradable polyanhydride, PBTPA (poly butanedithiol 1,3,5-triallyl-1,3,5-triazine-2,4,6(1H,3H,5H)-trione pentenoic anhydride), was synthesized and used as an organic encapsulant, capable of defining the lifecycle of device operation by controlling the water diffusion. Thiol-ene click-chemistry was used for the construction of degradable polyanhydride. Mixture of 1 mole of 4-pentenoic anhydride (4PA), 4 mole of 1,3,5-triallyl-1,3,5-triazine-2,4,6(1H,3H,5H)-trione (TTT) and 7 mole of 1,4-butanedithiol was polymerized by UV light for an hour with the addition of 2-hydroxy-4′-(2-hydroxyethoxy)-2-methylpropiophenone as the photoinitiator (total mass of 0.1%), yielding biodegradable PBTPA with hydrophobic chains (see FIG. 28). Here, 4PA and TTT act as a degradable linker and hydrophobic component, respectively, and butanedithiol crosslinks both 4PA and TTT through UV-induced thiol-ene reaction.

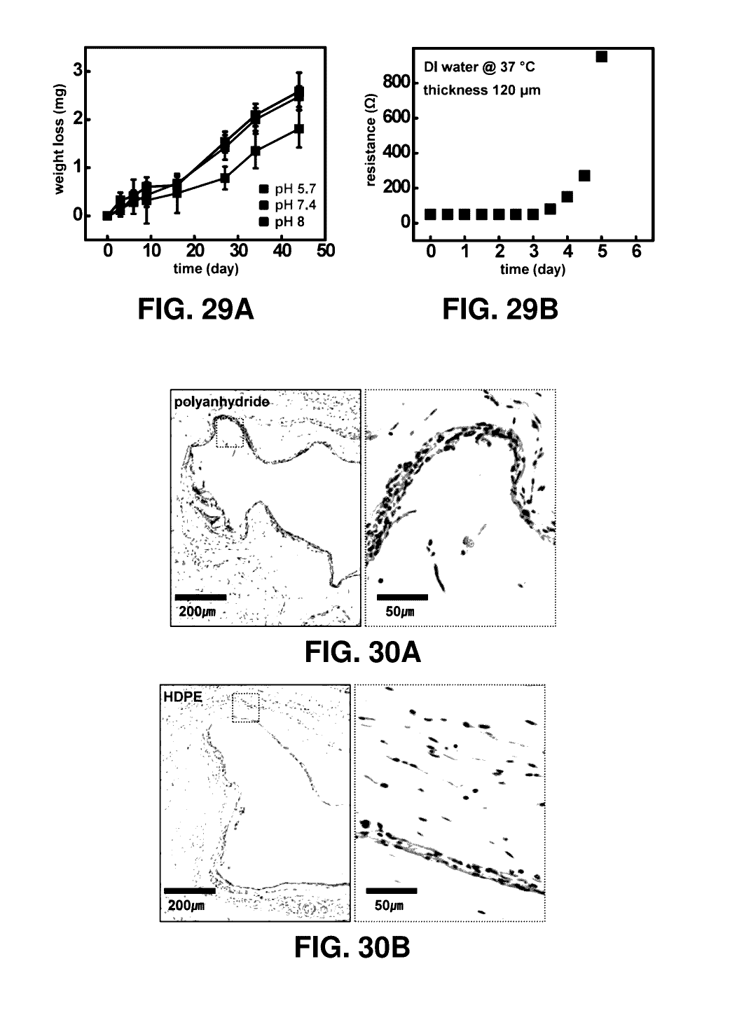

FIG. 29A shows the hydrolysis kinetics of polyanhydride at the different solution pH at room temperature, 0.5, 1.2, 2.8 mg/day for pH 6.7, 7.4, and 8 solutions. Since anhydride bonds can be hydrolyzed giving two hydrophilic carboxylic acid bonds, PBTPA tends to gradually collapse and dissolve into water (see FIG. 28). The water permeability can be tracked by a simple water-sensitive electrical element, i.e. an Mg resistor in this study. The resistance of the Mg resistor increases if the water diffuses to the Mg and begins to dissolve it. Details of this type of test method appear elsewhere. FIG. 29B shows the resistance at various times for a Mg resistor (˜300 nm thick) encapsulated with polyanhydride (˜120 μm). The Resistance is stable until 4 days and then starts to increase significantly, implying the Mg resistor has dissolved. Polyanyhdride provides a barrier to water diffusion to control the operating lifetime of the pressure sensor. This type of electrical degradation is observed in the encapsulation of the interconnecting electrodes (Mo). FIG. 32 shows the degradation of electrical function of biodegradable wires (Mo, Mg) and interconnecting electrode (Mo) measured without encapsulation. The Mg wires are stable for about 5 days (change of less than 3Ω) but then shows large changes in resistance, due to their increased rate of dissolution compared to Mo (which shows a much smaller resistance change, ˜3Ω). The Mo electrode parts dissolve within a few hours without encapsulation. Polyanhydride encapsulation enhances the stable operation time of Mo to six days with less than 10Ω change. At 7 days the water begins to dissolve the Mo interconnection significantly and the resistance increases to ˜50Ω.

An additional layer of encapsulation on the PLGA membrane affects the mechanics of deformation, thereby changing the calibration. FIG. 27A indicates a change from 82 to 50 Ω/mmHg via the addition of a 120 μm thick coating of polyanhydride. FIG. 27B summarizes results of FEA simulation, consistent with this observation. The average strain in the Si-NM sensor reduces with the addition of the encapsulation. The balance between sensitivity and operational lifecycle can be managed through appropriate selection of trench geometry, Si-NM width, modulus and thickness of the membrane and the encapsulation layer, and the water permeation (chemistry and thickness of encapsulation).

Pieces of polyanhydride (10 mm×3 mm) and HDPE (High-density polyethylene, negative control sample) were implanted subcutaneously in Balb/c mice to assess the toxicity. FIG. 30 provides hematoxylin and eosin (H&E) images of tissue around the implant sites for polyanhydride and HDPE after 14 days. Histologic examination showed that inflammatory cell infiltration and fibrosis in surrounding tissues were no different with negative control groups. There were no obvious signs of local toxicity caused by polyanhydride or its by-products as results were comparable to the HDPE group.

9. Immunohistochemistry

Immunohistochemistry was performed as described previously, with minor modifications. The two types of bioresorbable pressure sensors (np-Si and Mg foil structure) were placed on the cortex in the craniectomy site on the right hemisphere above the sensory motor cortex for 2 weeks, 4 weeks, and 8 weeks. A craniectomy is performed on the left side again above the sensorimotor cortex and the dura is opened above the sensorimotor cortex but no implant is placed. This acts as a histological control to the right side with the implant placed. Brain slices are double-immunostained for GFAP (glial fibrillary acidic protein) to detect astrocytes and Iba1 (ionized calcium-binding adapter molecule 1) to identify microglia/macrophages. Briefly, rats are anesthetized and intracardially perfused with ice-cold 4% paraformaldehyde in phosphate buffer (PB). Brains are carefully dissected with particular attention to preserve the right cortical surface beneath the now fully resorbed device and the left cortical surface acting as a control. Brains are post-fixed 2 hr at 4° C. and cryoprotected with solution of 30% sucrose in 0.1M PB at 4° C. for at least 4 d, cut into 30 μm sections and processed for immunostaining. 30 μm brain sections are washed three times in PBS and blocked for one hour in PBS containing 0.5% Triton X-100 and 5% normal goat serum. Sections are then incubated for 16 hr at 4° C. in guinea pig anti-GFAP (1:500, Synaptic Systems 173 004) and rabbit anti-Iba1 (1:300, Wako Chemicals 019-19741). Following incubation, sections are washed three times in PBS and then incubated for 2 hr at room temperature in Alexa fluor 488 goat anti-rabbit IgG (1:1000, Life Technologies A11008) and Alexa fluor 546 goat anti-guinea pig IgG (1:1000, Life Technologies A11074). Sections are then washed three times in PBS and then incubated for 1 hr at room temperature in NeuroTrace® 435/455 Blue Fluorescent Nissl Stain (Life Technologies N21479) in PB (1:400). Sections are then washed three times in PBS followed by three washes in PB and mounted on glass slides with HardSet Vectashield (Vector Labs) for microscopy. All sections imaged on a digital slide scanner (Olympus NanoZoomer HT model). Gain and exposure time are digitally set and constant throughout. Images corresponding with the right cortical surface interfacing with the device are compared with the left control cortical surface which did not have a device implanted. Gliosis is compared between cortex underlying the now resorbed implant and the control.

10. Other Applications of Bioresorbable Pressure Sensors

Sensors implanted at the intra-abdominal cavity and lower extremities demonstrated the versatility usages in implantable biomedical applications. The abdominal wall was shaved and prepped at the incision site with 70% ethanol. Animals were subjected to a 4 cm full-thickness median incision along the linea alba, using the xiphoid process as the reference point by a sterile technique. After abdominal incision, the transient pressure sensor and clinical pressure sensor were placed in the abdominal cavity amongst the abdominal viscera. A modified roman sandal technique using a 3/0 VICRYL RAPIDE (polyglactin 910) suture secured the sensor and the abdomen was closed with simple interrupted 3/0 VICRYL RAPIDE (polyglactin 910) sutures. Manual abdominal compression yielded the increase of intra-abdominal pressure. A small (˜5 mm) vertical incision was made along the thigh using scissors, and the skin was retracted laterally to implant the pressure sensor into extremities cavity. The muscles of the posterior thigh (including the hamstring muscles) were split, and the biodegradable pressure sensor and commercial sensor were placed in the musculofascial compartment. The implants were secured utilizing the same method as the abdominal surgery.

11. Injectable Biodegradable Sensors

A three-axis vertical stereotactic frame and arm temporary tied with an injectable device allowed accurate positioning and stable injection into the deep brain site. Adjustments of the jig enabled the needle system to penetrate into brain tissue via a square hole in the skull of a rat. Release of the needle device completed implantation, followed by sealing the skull with a silicone polymer (Kwik-Sil, World Precision Instruments Inc., USA) and dental acrylic. A manual increase in pressure induced by compressing the abdomen provided a means to compare the bioresorbable needle pressure sensor to a commercial device injected at an adjacent location. Monitoring the temperature during the anesthetization and recovery yielded data to compare the bioresorbable temperature sensor to temperatures measured by infrared imaging on the surface of brain.

12. Characterizations of Porosity and Average Pore Size of Nanoporous Silicon (np-Si)

The porosity of the np-Si was characterized by optical reflectance spectroscopy. The refractive index of np-Si (n) was determined as ˜1.55 from the spacing of adjacent maxima in the Fabry-Perot oscillations in the reflectance spectrum (˜1.3 to 1.6 μm) using

$$n = \frac{1}{2 d} \cdot \frac{1}{( \frac{1}{{λ}_{1}} - \frac{1}{{λ}_{2}} )} , \qquad ( 6 )$$

where λ₁ and λ₂ are the local maximum and the adjacent, longer-wavelength local maximum in the reflectance spectrum, and d is the thickness of np-Si determined by scanning electron microscope (SEM). The porosity was determined by using the calculated n and the two-component Bruggeman effective medium approximation:

$$φ ( \frac{1 - {n}^{2}}{1 + 2 {n}^{2}} ) + ( 1 - φ ) ( \frac{{n}_{Si}^{2} - {n}^{2}}{{n}_{Si}^{2} + 2 {n}^{2}} ) = 0 \qquad ( 7 )$$

where φ and n_{Si} are the porosity of np-Si and the real part of the refractive index of bulk Si, respectively. Assuming a Si bulk refractive index of 3.5, the porosity of np-Si is ˜70.5%. This agrees well with gravimetric porosity measurements (i.e. relative mass of np-Si film to mass of bulk Si that would occupy the same geometric volume), which suggest an average porosity of 69±3%.

The average pore size was determined using a gas sorption system (NOVA—e Series, Quantachrome Instruments). np-Si membranes were subjected to both nitrogen adsorption and desorption measurements. The sorption system's NovaWin software applied the Barrett, Joyner, and Halenda (BJH) Method to the experimental isotherms, resulting in an average pore size of 12±3 nm, which is in agreement with what is observed in the top view image from the scanning electron microscope.

### REFERENCES

1. Ning, H. et al. Transfer-Printing of Tunable Porous Silicon Microcavities with Embedded Emitters. ACS Photonics 1, 1144-1150 (2014).
2. Wu, J. et al. Mechanics of reversible adhesion. Soft Matter 7, 8657-8662 (2011).
3. Liu, C. Foundations of MEMS Ch. 6 (Prentice Hall, London, 2011).
4. Sugiyama, S., Takigawa, M., Igarashi, I. Integrated Piezoresistive Pressure Sensor with Both Voltage and Frequency Output. Sens. and Actuators A 4, 113-120 (1984).
5. Wang, C.-T., Nonlinear large-deflection boundary-value problems of rectangular plates. (DTIC Document, 1984).
6. Angell, J. B., Terry, S. C., Barth, P. W. Silicon Micromechanical Devices. Sci. Am. J. 248, 44-55 (1983).
7. Hsu, T. R. MEMS & Microsystems: Design, Manufacture, and Nanoscale Engineering Ch. 7 (John Wiley & Sons, Inc., Hoboken, N.J., USA, 2008).
8. Van der Horst, A. et al. A Novel Flexible Thermoelectric Sensor for Intravascular Flow Assessment. IEEE Sens. J. 13, 3883-3891 (2013).
9. Webb, R. C. et al. Ultrathin Conformal Devices for Precise and Continuous Thermal Characterization of Human Skin. Nature Mater. 12, 938-944 (2013).
10. Vezenov, D. V., Noy, A., Rozsnyai, L. F., Lieber, C. M. Force titrations and ionization state sensitive imaging of functional groups in aqueous solutions by chemical force microscopy. J. Am. Chem. Soc. 119, 2006-2015 (1997).
11. Cui, Y.; Wei, Q. Q.; Park, H. K.; Lieber, C. M. Nanowire Nanosensors for Highly-Sensitive, Selective and Integrated Detection of Biological and Chemical Species. Science 293, 1289-1292 (2001).
12. Penner, R. M. Chemical sensing with nanowires. Annu. Rev. Anal. Chem. 5, 461-485 (2012).
13. Hwang, S.-W. et al. A Physically Transient Form of Silicon Electronics. Science 337, 1640-1644 (2012).
14. Kang, S.-K. et al. Dissolution Behaviors and Applications of Silicon Oxides and Nitrides in Transient Electronics. Adv. Func. Mater. 24, 4427-4434 (2014).
15. M. R. Bruchas et al., Selective p38α MAPK deletion in serotonergic neurons produces stress resilience in models of depression and addiction. Neuron 71, 498-511 (2011).
16. Kim, T. I. et al. Injectable, Cellular-Scale Optoelectronics with Applications for Wireless Optogenetics. Science 240, 211-216 (2013).
17. Gaber, N., Khalil, D. & Shaarawi, A. Fabrication of optical filters using multilayered porous silicon. in Advanced Fabrication Technologies for Micro/Nano Optics and Photonics IV 7927, 79270S-79270S-9 (2011).
18. Bosch, S., Ferré-Borrull, J., Leinfellner, N. & Canillas, A. Effective dielectric function of mixtures of three or more materials: a numerical procedure for computations. Surf. Sci. 453, 9-17 (2000).
19. Schmid, P. E. Optical absorption in heavily doped silicon. Phys. Rev. B 23, 5531-5536 (1981).
20. Barrett, E. P., Joyner, L. G. & Halenda, P. P. The Determination of Pore Volume and Area Distributions in Porous Substances. I. Computations from Nitrogen Isotherms. J. Am. Chem. Soc. 73, 373-380 (1951).
21. Kang, S. K. et al. Biodegradable Thin Metal Foils and Spin-On Glass Materials for Transient Electronics, Adv. Func. Mater. 25, 1789-1797 (2015).
22. Yin, Y. et al. Dissolvable Metals for Transient Electronics. Adv. Func. Mater. 24, 645-658 (2014).
1. Hwang, S.-W. et al. A physically transient form of silicon electronics. Science 337, 1640-4 (2012).
2. Hwang, S.-W. et al. Materials and Fabrication Processes for Transient and Bioresorbable High-Performance Electronics. Adv. Funct. Mater. 23, 4087-4093 (2013).
3. Dagdeviren, C. et al. Transient, Biocompatible Electronics and Energy Harvesters Based on ZnO. Small 9, 3398-404 (2013).
4. Hwang, S.-W. et al. Materials for bioresorbable radio frequency electronics. Adv. Mater. 25, 3526-31 (2013).
5. Li, R. et al. An Analytical Model of Reactive Diffusion for Transient Electronics. Adv. Funct. Mater. 23, 31063114 (2013).
6. Yin, L. et al. Dissolvable Metals for Transient Electronics. Adv. Funct. Mater. 24, 645-658 (2014).
7. US 2011/0230747A1 (Sep. 22, 2011)—IMPLANTABLE BIOMEDICAL DEVICES ON BIORESORBABLE SUBSTRATES
8. US 2014/0163390A1 (Jun. 12, 2014)—IMPLANTABLE BIOMEDICAL DEVICES ON BIORESORBABLE SUBSTRATES
9. US 2013/0140649A1 (Jun. 6, 2013)—TRANSIENT DEVICES DESIGNED TO UNDERGO PROGRAMMABLE TRANSFORMATIONS
10. WO 2014/138465 (Sep. 12, 2014)—PROCESSING TECHNIQUES FOR SILICON-BASED TRANSIENT DEVICES
11. US 2014/0323968A1 (Oct. 30, 2014)—MATERIALS, ELECTRONIC SYSTEMS AND MODES FOR ACTIVE AND PASSIVE TRANSIENCE
12. US 2014/0305900A1 (Oct. 16, 2014)—TRANSIENT ELECTRONIC DEVICES COMPRISING INORGANIC OR HYBRID INORGANIC AND ORGANIC SUBSTRATES AND ENCAPSULATES
13. WO2013154606 A1/US20120265028 A1—Sensor, circuitry, and method for wireless intracranial pressure monitoring
14. WO2013041973 A2—Non-invasive intracranial pressure system

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. Specific names of compounds or components are intended to be exemplary, as it is known that one of ordinary skill in the art can name the same compounds or components differently.

The following references relate generally to fabrication methods, structures and systems for making electronic devices, and are hereby incorporated by reference to the extent not inconsistent with the disclosure in this application.

| Attorney | | | Publication | Publication | | |
|---|---|---|---|---|---|---|
| Docket No. | Application No. | Filing Date | No. | Date | Pat. No. | Issue Date |
| 145-03 US | 11/001,689 | Dec. 1, 2004 | 2006/0286488 | Dec. 21, 2006 | 7,704,684 | Apr. 27, 2010 |
| 18-04 US | 11/115,954 | Apr. 27, 2005 | 2005/0238967 | Oct. 27, 2005 | 7,195,733 | Mar. 27, 2007 |
| 38-04A US | 11/145,574 | Jun. 2, 2005 | 2009/0294803 | Dec. 3, 2009 | 7,622,367 | Nov. 24, 2009 |
| 38-04B US | 11/145,542 | Jun. 2, 2005 | 2006/0038182 | Feb. 23, 2006 | 7,557,367 | Jul. 7, 2009 |
| 43-06 US | 11/421,654 | Jun. 1, 2006 | 2007/0032089 | Feb. 8, 2007 | 7,799,699 | Sep. 21, 2010 |
| 38-04C US | 11/423,287 | Jun. 9, 2006 | 2006/0286785 | Dec. 21, 2006 | 7,521,292 | Apr. 21, 2009 |
| 41-06 US | 11/423,192 | Jun. 9, 2006 | 2009/0199960 | Aug. 13, 2009 | 7,943,491 | May 17, 2011 |
| 25-06 US | 11/465,317 | Aug. 17, 2006 | — | — | — | — |
| 137-05 US | 11/675,659 | Feb. 16, 2007 | 2008/0055581 | Mar. 6, 2008 | — | — |
| 90-06 US | 11/782,799 | Jul. 25, 2007 | 2008/0212102 | Sep. 4, 2008 | 7,705,280 | Apr. 27, 2010 |
| 134-06 US | 11/851,182 | Sep. 6, 2007 | 2008/0157235 | Jul. 3, 2008 | 8,217,381 | Jul. 10, 2012 |
| 151-06 US | 11/585,788 | Sep. 20, 2007 | 2008/0108171 | May 8, 2008 | 7,932,123 | Apr. 26, 2011 |
| 216-06 US | 11/981,380 | Oct. 31, 2007 | 2010/0283069 | Nov. 11, 2010 | 7,972,875 | Jul. 5, 2011 |
| 116-07 US | 12/372,605 | Feb. 17, 2009 | — | — | — | — |
| 213-07 US | 12/398,811 | Mar. 5, 2009 | 2010/0002402 | Jan. 7, 2010 | 8,552,299 | Oct. 8, 2013 |
| 38-04D US | 12/405,475 | Mar. 17, 2009 | 2010/0059863 | Mar. 11, 2010 | 8,198,621 | Jun. 12, 2012 |
| 170-07 US | 12/418,071 | Apr. 3, 2009 | 2010/0052112 | Mar. 4, 2010 | 8,470,701 | Jun. 25, 2013 |
| 216-06A US | 12/522,582 | Jul. 9, 2009 | — | — | — | — |
| 38-04A1 US | 12/564,566 | Sep. 22, 2009 | 2010/0072577 | Mar. 25, 2010 | 7,982,296 | Jul. 19, 2011 |
| 71-07 US | 12/669,287 | Jan. 15, 2010 | 2011/0187798 | Aug. 4, 2011 | — | — |
| 60-09 US | 12/778,588 | May 12, 2010 | 2010/0317132 | Dec. 16, 2010 | — | — |
| 43-06A US | 12/844,492 | Jul. 27, 2010 | 2010/0289124 | Nov. 18, 2010 | 8,039,847 | Oct. 18, 2011 |
| 15-10 US | 12/892,001 | Sep. 28, 2010 | 2011/0230747 | Sep. 22, 2011 | 8,666,471 | Mar. 4, 2014 |
| 19-10 US | 12/916,934 | Nov. 1, 2010 | 2012/0105528 | May 3, 2012 | 8,562,095 | Oct. 22, 2013 |
| 3-10 US | 12/947,120 | Nov. 16, 2010 | 2011/0170225 | Jul. 14, 2011 | — | — |
| 118-08 US | 12/996,924 | Dec. 8, 2010 | 2011/0147715 | Jun. 23, 2011 | 8,946,683 | Feb. 3, 2015 |
| 126-09 US | 12/968,637 | Dec. 15, 2010 | 2012/0157804 | Jun. 21, 2012 | — | — |
| 50-10 US | 13/046,191 | Mar. 11, 2011 | 2012/0165759 | Jun. 28, 2012 | — | — |
| 151-06A US | 13/071,027 | Mar. 24, 2011 | 2011/0171813 | Jul. 14, 2011 | — | — |
| 137-05A US | 13/095,502 | Apr. 27, 2011 | — | — | — | — |
| 216-06B US | 13/100,774 | May 4, 2011 | 2011/0266561 | Nov. 3, 2011 | 8,722,458 | May 13, 2014 |
| 38-04A2 US | 13/113,504 | May 23, 2011 | 2011/0220890 | Sep. 15, 2011 | 8,440,546 | May 14, 2013 |
| 136-08 US | 13/120,486 | Aug. 4, 2011 | 2011/0277813 | Nov. 17, 2011 | 8,679,888 | Mar. 25, 2014 |
| 151-06B US | 13/228,041 | Sep. 8, 2011 | 2011/0316120 | Dec. 29, 2011 | — | — |
| 43-06B US | 13/270,954 | Oct. 11, 2011 | 2012/0083099 | Apr. 5, 2012 | 8,394,706 | Mar. 12, 2013 |
| 3-11 US | 13/349,336 | Jan. 12, 2012 | 2012/0261551 | Oct. 18, 2012 | — | — |
| 38-04E US | 13/441,618 | Apr. 6, 2012 | 2013/0100618 | Apr. 25, 2013 | 8,754,396 | Jun. 17, 2014 |
| 134-06B US | 13/441,598 | Apr. 6, 2012 | 2012/0327608 | Dec. 27, 2012 | 8,729,524 | May 20, 2014 |
| 28-11 US | 13/472,165 | May 15, 2012 | 2012/0320581 | Dec. 20, 2012 | — | — |
| 7-11 US | 13/486,726 | Jun. 1, 2012 | 2013/0072775 | Mar. 21, 2013 | 8,934,965 | Jan. 13, 2015 |
| 29-11 US | 13/492,636 | Jun. 8, 2012 | 2013/0041235 | Feb. 14, 2013 | — | — |
| 84-11 US | 13/549,291 | Jul. 13, 2012 | 2013/0036928 | Feb. 14, 2013 | — | — |
| 25-06A US | 13/596,343 | Aug. 28, 2012 | 2012/0321785 | Dec. 20, 2012 | 8,367,035 | Feb. 5, 2013 |
| 150-11 US | 13/624,096 | Sep. 21, 2012 | 2013/0140649 | Jun. 6, 2013 | — | — |
| 38-04A3 US | 13/801,868 | Mar. 13, 2013 | 2013/0320503 | Dec. 5, 2013 | 8,664,699 | Mar. 4, 2014 |
| 125-12 US | 13/835,284 | Mar. 15, 2013 | 2014/0220422 | Aug. 7, 2014 | — | — |
| 30-13 US | 13/853,770 | Mar. 29, 2013 | 2013/0333094 | Dec. 19, 2013 | — | — |
| 213-07A US | 13/974,963 | Aug. 23, 2013 | 2014/0140020 | May 22, 2014 | — | — |
| 19-10A US | 14/033,765 | Sep. 23, 2013 | 2014/0092158 | Apr. 3, 2014 | — | — |
| 15-10A US | 14/140,299 | Dec. 24, 2013 | 2014/0163390 | Jun. 12, 2014 | — | — |
| 38-04A4 US | 14/155,010 | Jan. 14, 2014 | 2014/0191236 | Jul. 10, 2014 | — | — |
| 136-08A US | 14/173,525 | Feb. 5, 2014 | 2014/0216524 | Aug. 7, 2014 | — | — |
| 216-06C US | 14/209,481 | Mar. 13, 2014 | 2014/0373898 | Dec. 25, 2014 | — | — |
| 134-06C US | 14/220,910 | Mar. 20, 2014 | 2014/0374872 | Dec. 25, 2014 | — | — |
| 38-04F US | 14/220,923 | Mar. 20, 2014 | 2015/0001462 | Jan. 1, 2015 | — | — |
| 151-06C US | 14/246,962 | Apr. 7, 2014 | 2014/0361409 | Dec. 11, 2014 | — | — |
| 62-13 US | 14/250,671 | Apr. 11, 2014 | 2014/0305900 | Oct. 16, 2014 | — | — |
| 56-13 US | 14/251,259 | Apr. 11, 2014 | 2014/0323968 | Oct. 30, 2014 | — | — |
| 60-09A US | 14/479,100 | Sep. 5, 2014 | — | — | — | — |
| 84-13 US | 14/504,736 | Oct. 2, 2014 | — | — | — | — |
| 213-07B US | 14/521,319 | Oct. 22, 2014 | — | — | — | — |
| 78-11A US | 14/532,687 | Nov. 4, 2014 | 2015/0080695 | Mar. 19, 2015 | — | — |
| 2-14 US | 14/599,290 | Jan. 16, 2015 | — | — | — | — |
| 71-07A US | 14/686,304 | Apr. 14, 2015 | — | — | — | — |

Every formulation or combination of components described or exemplified herein can be used to practice the invention, unless otherwise stated.

Whenever a range is given in the specification, for example, a size range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when compositions of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described.

1. An implantable and bioresorbable sensor comprising:
a substrate;
an electronic device supported by the substrate, wherein the electronic device comprises a semiconductor or metallic component;
a barrier layer that isolates the electronic device from a surrounding environment during use; and
wherein each of the substrate, electronic device, and barrier layer have a bioresorption rate during use to provide controlled bioresorption of the implantable and bioresorbable sensor, configured so that the implantable and bioresorbable sensor operates for an operational lifetime and has a bioresorption lifetime that is greater than the operational lifetime, and no detectable portion of the implantable and bioresorbable sensor remains at an implantation site after the bioresorption lifetime.

2. The implantable and bioresorbable sensor of claim 1, wherein the barrier layer fluidically, electrically and chemically isolates the electronic device from a surrounding fluid during use.

3. (canceled)

4. The implantable and bioresorbable sensor of claim 1, wherein the semiconductor or metallic component has a thickness less than or equal to 10 μm.

5. The implantable and bioresorbable sensor of claim 1, wherein the electronic device comprises a sensor selected from the group consisting of: a pressure sensor; a temperature sensor; a motion sensor; a flow sensor; a thermal conductivity sensor; a diffusivity sensor; a pH sensor; an electrical sensor; an optical sensor; a glucose sensor, oxygen sensor and a biomolecule sensor.

6. The implantable and bioresorbable sensor of claim 1 that is multi-functional, with the electronic device comprising a first electronic device that senses a first physical parameter and a second electronic device that senses a second physical parameter, wherein the physical parameters are selected from the group consisting of pressure, temperature, acceleration, electric potential, impedance, fluid flow-rate, thermal conductivity, pH, glucose level, oxygen level, and biomolecule detection.

7. The implantable and bioresorbable sensor of claim 1, wherein the electronic device comprises a pressure sensor, the pressure sensor comprising:
a cavity disposed in the substrate;
a deformable layer supported by the substrate and that covers the cavity; and
a strain gauge supported by the deformable layer and adjacent to the cavity, wherein deflection of the deformable layer by a pressure in a fluid surrounding the deformable layer and strain gauge generates an electrical output from the pressure sensor strain gauge that depends on the fluid pressure.

8. The implantable and bioresorbable sensor of claim 7, wherein the strain gauge is made from a material selected from the group consisting of: a Si-nanomembrane; a polycrystalline Si; germanium; silicon-germanium; and a metal or alloy thereof.

9-11. (canceled)

12. The implantable and bioresorbable sensor of claim 7, wherein the stain gauge comprises a Si-nanomembrane with a distal piezoresistive end having a serpentine geometry and a cavity volume that is greater than 0.1 mm³ and less than 2 mm³.

13-14. (canceled)

15. The implantable and bioresorbable sensor of claim 1, further comprising an at least partially resorbable wireless data-transmitter electrically connected to the electronic device, wherein the electronic device further comprises:
a near-field communication (NFC) microchip;
an electronic circuit comprising:
electrical interconnects, resistors, and capacitors that electrically connect the NFC chip and semiconductor or metallic component for data acquisition, processing and transmission; and
an inductive coil or radiofrequency receiver for powering the implantable and bioresorbable sensor from an externally-positioned or subdermally-implanted electronic component.

16-22. (canceled)

23. The implantable and bioresorbable sensor of claim 1, further comprising an encapsulation layer that encapsulates the implantable and bioresorbable sensor, the encapsulation layer comprising a bioresorbable polymer layer, a bioresorbable inorganic layer, or both a bioresorbable polymer layer and a bioresorbable inorganic layer, wherein the bioresorbable polymer layer and the bioresorbable inorganic layer have a thickness selected to provide a desired operational lifetime.

24. (canceled)

25. The implantable and bioresorbable sensor of claim 1, further comprising:
an electrical interconnect;
a wireless transmitter, wherein the electrical interconnect electrically connects the wireless transmitter to the electronic device; and
wherein the electrical interconnect comprises a bioresorbable metal wire having a thickness less than 50 μm and a width less than 500 μm.

26. The implantable and bioresorbable sensor of claim 1 wherein the operational lifetime is greater than or equal to one week, wherein for the bioresorption lifetime that is greater than the operational lifetime, each of the substrate, the barrier layer and the electronic device are configured for bioresorption by a patient in which the implantable and bioresorbable sensor is implanted.

27. The implantable and bioresorbable sensor of claim 26, wherein constituents of the implantable and bioresorbable sensor are configured to dissolve by any one or more of hydrolysis, enzyme reaction, or metabolic reaction after implantation in a programmed timeframe.

28. The implantable and bioresorbable sensor of claim 27, wherein the constituents are configured to dissolve within 14 months after implantation.

29. The implantable and bioresorbable sensor of claim 28, further comprising electrical interconnects to electrically connect the electronic device to a wireless transmitter, wherein the electrical interconnects comprise biodegradable metal wires having a thickness less than 50 μm and configured to have a dissolution rate when in contact with a biofluid during use that is between 0.2 nm/day and 20 nm/day.

30. The implantable and bioresorbable sensor of claim 29, further comprising a polymer layer supported by the substrate, the polymer layer configured to have a dissolution rate when in contact with a biofluid such that after a time period that is greater than three weeks the polymer layer has dissolved.

31. The implantable and bioresorbable sensor of claim 29, wherein a barrier layer composition and a barrier layer thickness are selected to provide a desired operational lifetime that is between 1 day and 12 months.

32-34. (canceled)

35. The implantable and bioresorbable sensor of claim 1, for monitoring two or more parameters selected from the group consisting of: pressure; temperature; motion; fluid flow; conductivity; diffusivity; pH; oxygen level; electric potential; impedance; optical image; presence of a biomolecule of interest; glucose presence or concentration; and lactate presence or concentration.

36. The implantable and bioresorbable sensor of claim 1, configured for implantation into a cavity of a living animal, wherein after a bioresorption lifetime, no detectable portion of the implantable and bioresorbable sensor remains in the living animal.

37. The implantable and bioresorbable sensor of claim 36, configured to adhere to a tissue, body cavity or organ wall selected from the group consisting of: a luminal facing blood vessel wall; an external-facing blood vessel wall, brain, heart, lung, eye, esophagus, stomach, sphincter, liver, urinary bladder, and spinal cord.

38. (canceled)

39. An implantable and bioresorbable pressure sensor comprising:
a substrate;
a cavity disposed in the substrate;
a deformable layer supported by the substrate and that covers the cavity;
an electronic device supported by the substrate, wherein the electronic device comprises a strain gauge supported by the deformable layer and adjacent to the cavity, wherein deflection of the deformable layer by a fluid pressure in a fluid surrounding a top surface of the deformable layer and strain gauge generates an electrical output from the strain gauge that depends on a magnitude of the fluid pressure; and
a barrier layer that fluidically isolates the electronic device during use.

40. The implantable and bioresorbable pressure sensor of claim 39, wherein the deformable layer comprises a polymer layer, an inorganic layer, or both a polymer and inorganic layer.

41-42. (canceled)

43. The implantable and bioresorbable pressure sensor of claim 39, wherein the strain gauge comprises a bioresorbable nanomembrane formed of: Si, polycrystalline Si, Ge, Si—Ge, a-IGZO, Mg, Mo, Zn, W, Fe, or alloys thereof, wherein the nanomembrane has a thickness less than 1 μm.

44. The implantable and bioresorbable pressure sensor of claim 43 that is a Si-nanomembrane strain gauge that comprises a distal piezoresistive end having a serpentine geometry.

45. The implantable and bioresorbable pressure sensor of claim 39, further comprising an additional sensor electrically connected to the electronic device to provide a multi-functional sensor that detects pressure and at least one additional physical parameter.

46. An extraction-free method of remotely sensing one or more physical parameters in a living animal, the method comprising the steps of:
inserting into a living animal an implantable and bioresorbable sensor operably connected to a wireless transmitter;
electrically coupling the inserted implantable and bioresorbable sensor to an externally located electronic component to power the sensor and wireless transmitter;
outputting to the wireless transmitter from the implantable and bioresorbable sensor an electrical output, wherein the electrical output depends on the one or more physical parameters;
wirelessly transmitting the electrical output to the externally located electronic component; and
hydrolyzing the implantable and bioresorbable sensor so that after an operational lifetime no observable or detectable portion of the implantable and bioresorbable sensor remains in the living animal,
thereby providing the extraction-free method of remotely sensing one or more physical parameters.
